(19) United States
(12) Patent Application Publication
(10) Pub. No.: US 2024/0216589 A1
(43) Pub. Date: Jul. 4, 2024

(54) DRUG DELIVERY COMPOSITIONS AND METHODS OF CONTROLLING DRUG DELIVERY RATES OF SUBCUTANEOUS SENSORS

(71) Applicant: ABBOTT DIABETIS CARE INC., Alameda, CA (US)

(72) Inventors: Soya J. Gamsey, Brisbane, CA (US); Catherine SNYDER, San Leandro, CA (US); Udo HOSS, San Ramon, CA (US)

(73) Assignee: ABBOTT DIABETIS CARE INC., aLAMEDA, CA (US)

(21) Appl. No.: 18/402,684

(22) Filed: Jan. 2, 2024

Related U.S. Application Data
(60) Provisional application No. 63/477,977, filed on Dec. 30, 2022.

(51) Int. Cl. A61L 31/16; A61B 5/1486; A61L 31/04

(52) U.S. Cl. CPC A61L 31/16; A61B 5/14865; A61L 31/048; A61L 2300/216

(57) ABSTRACT

The present disclosure provides a drug delivery composition and a method of controlling a drug delivery rate of an analyte sensor. The drug delivery composition includes a copolymer including a plurality of copolymer chains, where each of the plurality of copolymer chains comprises a backbone including a plurality of hydrophilic units and a plurality of hydrophobic units, a crosslinker, and a therapeutic agent, where the crosslinker crosslinks at least a portion of the hydrophilic units of respective copolymer chains to form charges, and the hydrophobic units of the copolymer interact with the therapeutic agent through the non-polar intermolecular interaction. The drug delivery composition continuously releases the therapeutic agent at a set or predetermined drug delivery rate for a set or predetermined period of time.

### CROSS REFERENCE TO RELATED APPLICATIONS

The present application claims priority to and the benefit of U.S. Provisional Application No. 63/477,977, filed on Dec. 30, 2022, the entire content of which being incorporated herein by reference in its entirety.

### FIELD

The present disclosure relates to drug delivery compositions and methods of controlling a drug delivery rate, for example, the present disclosure relates to an analyte sensor comprising a drug delivery composition and a method of controlling a drug delivery rate of an analyte sensor, e.g., a subcutaneous sensor. The present disclosure further provides analyte sensors comprising such drug delivery compositions to reduce sensor signal inaccuracies or in vivo sensor failure, e.g., due to foreign body response (FBR).

### BACKGROUND

The detection of one or more suitable analytes within an individual can sometimes be vital for monitoring the condition of their health as deviations from normal analyte levels can be indicative of a physiological condition. For example, monitoring glucose levels can enable people suffering from diabetes to take appropriate or suitable corrective action including administration of medicine or consumption of particular food or beverage products to avoid significant physiological harm. Other analytes can be desirable to monitor for other physiological conditions. In certain instances, it can be desirable to monitor more than one analyte to monitor multiple physiological conditions, particularly when a person is suffering from comorbid conditions that result in simultaneous dysregulation of two or more analytes in combination with one another.

Analyte monitoring in an individual can take place periodically or continuously over a period of time. Periodic analyte monitoring can take place by withdrawing a sample of bodily fluid, such as blood or urine, at set time intervals and analyzing ex vivo. Periodic, ex vivo analyte monitoring can be sufficient to determine the physiological condition of many individuals. However, ex vivo analyte monitoring can be inconvenient or painful in some instances. Moreover, there is no way to recover lost data when an analyte measurement is not obtained at an appropriate or suitable time. Continuous analyte monitoring can be conducted utilizing one or more sensors that remain at least partially implanted within a tissue of an individual, such as dermally, subcutaneously, or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well.

However, implantable sensors can be plagued by short life spans when implanted in vivo. For example, the in vivo loss of sensor function seen in implantable sensors is thought to be in large part the result of certain responses, including immune responses, inflammation, fibrosis, and vessel regression, that occur in the tissue around (e.g., surrounding) implanted sensors. These tissue responses can be the result of tissue trauma arising from the insertion of the sensor into the skin and can result from the tissue reacting to the sensor as a foreign body. Although the tissue response at sites of sensor implantation is histopathologically similar to other forms of tissue inflammation, the ability to utilize anti-inflammatory agents (e.g., glucocorticoids and nonsteroidal anti-inflammatory agents) and/or other therapeutic agents to suppress or reduce sensor induced tissue trauma directly has been limited, for example, suppressing sensor induced tissue trauma and other physiological responses during a lifetime of the implanted sensor. As such, there is a need in the art to develop a drug delivery composition including anti-inflammatory agents and/or other therapeutic agents and methods of delivering such therapeutic compositions near an analyte sensor over a period of time at a desired or suitable rate delivery rate.

### SUMMARY

The purpose and aspects of the disclosed subject matter will be set forth in and are apparent from the description that follows, as well as will be learned by practice of the disclosed subject matter. Additional aspects of the disclosed subject matter will be realized and attained by compositions, devices, and methods particularly pointed out in the written description and claims hereof, as well as from the appended drawings.

One or more aspects of embodiments of the present disclosure are directed toward a drug delivery composition. In certain embodiments, the drug delivery composition can include (i) a copolymer including a plurality of copolymer chains, wherein each of the plurality of copolymer chains comprises a backbone including a plurality of hydrophilic units and a plurality of hydrophobic units, (ii) a crosslinker crosslinking at least a portion of the hydrophilic units between respective copolymer chains, and (iii) a therapeutic agent.

In certain embodiments, the hydrophilic unit of the copolymer can include a nitrogen-containing heterocyclic unit such as a pyridine unit, a pyridazine unit, a pyrimidine unit, a pyrazine unit, a triazine unit, an imidazole unit, a pyrazole unit, etc. In certain embodiments, the hydrophobic unit of the copolymer can include a non-heteroatom containing aromatic unit (such as a benzene (phenyl) unit, a naphthalene unit, an anthracene unit, etc.), an acyclic aliphatic unit (such as a straight or branched alkyl unit), a straight or branched alkenyl unit, a straight or branched alkynyl unit, etc., and/or a cyclic aliphatic unit such (as a cyclobutyl, a cyclopentyl unit, a cyclohexyl unit, a cycloheptyl unit, a cyclooctyl unit, a cyclohexenyl unit, etc.).

In certain embodiments, the copolymer can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.

In certain embodiments, the copolymer can include a block polymer.

In certain embodiments, the copolymer is a polyvinylpyridine-based copolymer.

In certain embodiments, the polyvinylpyridine-based copolymer can be a copolymer of vinylpyridine and styrene or a derivative thereof.

In certain embodiments, the polyvinylpyridine-based copolymer can be a polyvinylpyridine-co-polystyrene polymer.

In certain embodiments, the polyvinylpyridine-co-polystyrene polymer can include about 1-50 mer % of styrene units. In certain embodiments, the polyvinylpyridine-co-polystyrene polymer can include about 1-40 mer % of styrene units. In certain embodiments, the polyvinylpyridine-co-polystyrene polymer can include about 1-30 mer % of styrene units.

In certain embodiments, a weight average molecular weight of the copolymer is in a range of about 5 kD-1,000 kD.

In certain embodiments, the crosslinker can be a diglycidyl- or triglycidyl-functional epoxy.

In certain embodiments, the crosslinker can be selected from the group consisting of diglycidyl-PEG (200-1000), glycerol triglycidyl ether, and a combination thereof.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, mol % crosslinking of the copolymer can be in a range of about 0.1 mol %-50 mol %. In certain embodiments, mol % crosslinking of the copolymer can be in a range of about 1 mol %-50 mol %. In certain embodiments, mol % crosslinking of the copolymer can be in a range of about 0.1 mol %-30 mol %. In certain embodiments, mol % crosslinking of the copolymer can be in a range of about 1 mol %-30 mol %. In certain embodiments, mol % crosslinking of the copolymer can be in a range of about 0.1 mol %-10 mol %. In certain embodiments, mol % crosslinking of the copolymer can be in a range of about 1 mol %-10 mol %.

In certain embodiments, the therapeutic agent can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.

In certain embodiments, the therapeutic agent can be an anti-inflammatory agent. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. In certain embodiments, the anti-inflammatory agent is dexamethasone or a derivative or a salt form thereof. In certain embodiments, the derivative and/or salt form of dexamethasone is dexamethasone acetate. In certain embodiments, the derivative and/or salt form of dexamethasone is dexamethasone sodium phosphate.

In certain embodiments, the drug delivery composition can include an amount of the therapeutic agent in a range of 0.01 wt %-50 wt % based on the total weight of the copolymer. In certain embodiments, the drug delivery composition can include an amount of the therapeutic agent in a range of 0.01 wt %-40 wt % based on the total weight of the copolymer.

In certain embodiments, the drug delivery composition can include about 0.1 μg to about 200 μg of the therapeutic agent. In certain embodiments, the drug delivery composition can include about 0.1 μg to about 20 μg of the therapeutic agent. In certain embodiments, the drug delivery composition can include about 0.1 μg to about 10 μg of the therapeutic agent.

In certain embodiments, the crosslinker is bonded to the hydrophilic unit of the copolymer to form a charge.

In certain embodiments, the therapeutic agent is not covalently bound to the copolymer.

In certain embodiments, the therapeutic agent is covalently bound to the copolymer.

In certain embodiments, the drug delivery composition can continuously release the therapeutic agent at a set or predetermined drug delivery rate for a set or predetermined day's such as for at least 30 days.

One or more aspects of embodiments of the present disclosure are directed toward an analyte sensor. In certain embodiments, the analyte sensor can include: (i) a sensor tail including at least a first working electrode: (ii) an active area disposed upon a surface of the first working electrode for detecting an analyte: (iii) a mass transport limiting membrane permeable to the analyte that overcoats at least the active area: (iv) a counter/reference electrode: and (v) a drug delivery composition including (a) a copolymer including a plurality of copolymer chains, wherein each of the plurality of copolymer chains comprises a backbone including a plurality of hydrophilic units and a plurality of hydrophobic units, (b) a crosslinker crosslinking at least a portion of the hydrophilic units between respective copolymer chains, and (c) a therapeutic agent.

In certain embodiments, the analyte is glucose. In certain embodiments, the analyte sensor is a dermal sensor. In certain embodiments, the analyte sensor is a subcutaneous sensor such as a subcutaneously implanted sensor. In certain embodiments, the analyte sensor is an intravenous sensor such as intravenously implanted sensor.

In certain embodiments, the hydrophilic unit of the copolymer of the drug delivery composition present on the analyte sensor can include a nitrogen-containing heterocyclic unit such as a pyridine unit, a pyridazine unit, a pyrimidine unit, a pyrazine unit, a triazine unit, an imidazole unit, a pyrazole unit, etc. In certain embodiments, the hydrophilic unit of the copolymer of the drug delivery composition present on the analyte sensor can include a pyridine unit.

In certain embodiments, the hydrophobic unit of the copolymer of the drug delivery composition present on the analyte sensor can include a non-heteroatom containing aromatic unit such as a benzene (phenyl) unit, a naphthalene unit, an anthracene unit, etc., an acyclic aliphatic unit such as a straight or branched alkyl unit, a straight or branched alkenyl unit, a straight or branched alkynyl unit, etc., and/or a cyclic aliphatic unit such as a cyclobutyl, a cyclopentyl unit, a cyclohexyl unit, a cycloheptyl unit, a cyclooctyl unit, a cyclohexenyl unit, etc. In certain embodiments, the hydrophobic unit of the copolymer of the drug delivery composition present on the analyte sensor can include a non-heteroatom containing aromatic unit.

In certain embodiments, the copolymer can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.

In certain embodiments, the copolymer can include a block polymer.

In certain embodiments, the copolymer can be a polyvinylpyridine-based copolymer. In certain embodiments, the polyvinylpyridine-based copolymer can be a copolymer of vinylpyridine and styrene or a derivative thereof.

In certain embodiments, the polyvinylpyridine-based copolymer can be a polyvinylpyridine-co-polystyrene polymer.

In certain embodiments, the polyvinylpyridine-co-polystyrene polymer can include about 1-50 mer % of styrene units. In certain embodiments, the polyvinylpyridine-co-polystyrene polymer can include about 1-40 mer % of styrene units. In certain embodiments, the polyvinylpyridine-co-polystyrene polymer can include about 1-30 mer % of styrene units.

In certain embodiments, a weight average molecular weight of the copolymer is in a range of about 5 kD-1,000 kD.

In certain embodiments, the crosslinker can be a diglycidyl- or triglycidyl-functional epoxy.

In certain embodiments, the crosslinker can be selected from the group consisting of diglycidyl-PEG (200-1000), glycerol triglycidyl ether, and a combination thereof.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, mol % crosslinking of the copolymer can be in a range of about 0.1 mol %-50 mol %. In certain embodiments, mol % crosslinking of the copolymer can be in a range of about 1 mol %-50 mol %. In certain embodiments, mol % crosslinking of the copolymer can be in a range of about 0.1 mol %-30 mol %. In certain embodiments, mol % crosslinking of the copolymer can be in a range of about 1 mol %-30 mol %. In certain embodiments, mol % crosslinking of the copolymer can be in a range of about 0.1 mol %-10 mol %. In certain embodiments, mol % crosslinking of the copolymer can be in a range of about 1 mol %-10 mol %.

In certain embodiments, the therapeutic agent present in the drug delivery composition on the analyte sensor can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.

In certain embodiments, the therapeutic agent can be an anti-inflammatory agent. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. In certain embodiments, the anti-inflammatory agent is dexamethasone or a derivative or a salt form thereof. In certain embodiments, the derivative and/or salt form of dexamethasone is dexamethasone acetate. In certain embodiments, the derivative and/or salt form of dexamethasone is dexamethasone sodium phosphate.

In certain embodiments, the drug delivery composition can include an amount of the therapeutic agent in a range of about 0.01 wt %-50 wt % based on the total weight of the copolymer. In certain embodiments, the drug delivery composition can include an amount of the therapeutic agent in a range of about 0.01 wt %-40 wt % based on the total weight of the copolymer.

In certain embodiments, the drug delivery composition can include about 0.1 μg to about 200 μg of the therapeutic agent. In certain embodiments, the drug delivery composition can include about 0.1 μg to about 20 μg of the therapeutic agent. In certain embodiments, the drug delivery composition can include about 0.1 μg to about 10 μg of the therapeutic agent.

In certain embodiments, the crosslinker is bonded to the hydrophilic unit of the copolymer to form a charge.

In certain embodiments, the drug delivery composition can be disposed on an electrode of an analyte sensor. In certain embodiments, the drug delivery composition can be disposed on the working electrode of an analyte sensor. In certain embodiments, the drug delivery composition can be disposed on the counter/reference electrode of an analyte sensor. In certain embodiments, the drug delivery composition can be disposed on the counter electrode of an analyte sensor. In certain embodiments, the drug delivery composition can be disposed on the reference electrode of an analyte sensor.

In certain embodiments, the drug delivery composition can be disposed on the mass transport limiting membrane of an analyte sensor.

In certain embodiments, the therapeutic agent is not covalently bound to the copolymer.

In certain embodiments, the therapeutic agent is covalently bound to the copolymer.

In certain embodiments, the drug delivery composition can continuously release the therapeutic agent at a set or predetermined drug delivery rate for a set or predetermined days such as for at least 30 days.

One or more aspects of embodiments of the present disclosure are directed toward a method of controlling a drug delivery rate of an analyte sensor, e.g., a subcutaneous sensor, comprising a drug delivery composition. In certain embodiments, the present disclosure provides methods of delivering an analyte sensor of the present disclosure. In certain embodiments, the method can include providing an analyte sensor disclosed herein, e.g., an analyte sensor comprising a drug delivery composition, and implanting the analyte sensor subcutaneously. Alternatively or additionally, a drug delivery composition can be inserted into the tissue of the subject in close proximity to the analyte sensor.

In certain embodiments, the method of controlling a drug delivery rate of an analyte sensor and/or the method of delivering an analyte sensor such as a subcutaneous sensor can include: (i) providing a sharp including an analyte sensor and a drug delivery composition including (a) a copolymer including a plurality of copolymer chains, wherein each of the plurality of copolymer chains comprises a backbone including a plurality of hydrophilic units and a plurality of hydrophobic units, (b) a crosslinker crosslinking at least a portion of the hydrophilic units between respective copolymer chains, and (c) a therapeutic agent, (ii) penetrating a tissue of a subject with the sharp, (iii) inserting the drug delivery composition and analyte sensor into the tissue of the subject and (iv) retracting the sharp from the tissue of the subject. In certain embodiments, the analyte sensor is positioned within a channel of the sharp and the drug delivery composition is positioned distally to the analyte sensor within the channel of the sharp.

In certain embodiments, the method of controlling a drug delivery rate of an analyte sensor and/or the method of delivering an analyte sensor such as a subcutaneous sensor can include: (i) providing a sharp including an analyte sensor comprising a drug delivery composition including (a) a copolymer including a plurality of copolymer chains, wherein each of the plurality of copolymer chains comprises a backbone including a plurality of hydrophilic units and a plurality of hydrophobic units, (b) a crosslinker crosslinking at least a portion of the hydrophilic units between respective copolymer chains, and (c) a therapeutic agent, (ii) penetrating a tissue of a subject with the sharp. (iii) inserting the analyte sensor into the tissue of the subject and (iv) retracting the sharp from the tissue of the subject. In certain embodiments, the sharp can further include a second drug delivery composition, e.g., a second drug delivery composition that is positioned distally to the analyte sensor within the channel of the sharp.

One or more aspects of embodiments of the present disclosure are directed toward a sharp, such as a pre-loaded sharp for delivering a drug delivery composition. In certain embodiments, the sharp can include a drug delivery composition disclosed herein. In certain embodiments, the sharp can include an analyte sensor and a drug delivery composition disclosed herein. For example, but not by way of limitation, the drug delivery composition includes (i) a copolymer including a plurality of copolymer chains, wherein each of the plurality of copolymer chains comprises a backbone including a plurality of hydrophilic units and a plurality of hydrophobic units, (ii) a crosslinker crosslinking at least a portion of the hydrophilic units between respective copolymer chains, and (iii) a therapeutic agent. In certain embodiments, the analyte sensor is positioned within a channel of the sharp and the drug delivery composition is positioned distally to the analyte sensor within the channel of the sharp.

In certain embodiments, the sharp can include an analyte sensor comprising a drug delivery composition disclosed herein. For example, but not by way of limitation, the drug delivery composition includes (i) a copolymer including a plurality of copolymer chains, wherein each of the plurality of copolymer chains comprises a backbone including a plurality of hydrophilic units and a plurality of hydrophobic units, (ii) a crosslinker crosslinking at least a portion of the hydrophilic units between respective copolymer chains, and (iii) a therapeutic agent. In certain embodiments, the sharp can further include a second drug delivery composition, e.g., a second drug delivery composition that is positioned distally to the analyte sensor within the channel of the sharp.

In certain embodiments, the hydrophilic unit of the copolymer can include a nitrogen-containing heterocyclic unit such as a pyridine unit, a pyridazine unit, a pyrimidine unit, a pyrazine unit, a triazine unit, an imidazole unit, a pyrazole unit, etc. In certain embodiments, the hydrophilic unit of the copolymer can include a pyridine unit. In certain embodiments, the hydrophobic unit of the copolymer can include a non-heteroatom containing aromatic unit such as a benzene (phenyl) unit, a naphthalene unit, an anthracene unit, etc., an acyclic aliphatic unit such as a straight or branched alkyl unit, a straight or branched alkenyl unit, a straight or branched alkynyl unit, etc., and/or a cyclic aliphatic unit such as a cyclobutyl, a cyclopentyl unit, a cyclohexyl unit, a cycloheptyl unit, a cyclooctyl unit, a cyclohexenyl unit, etc. In certain embodiments, the hydrophobic unit of the copolymer can include an aromatic unit.

In certain embodiments, the copolymer can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.

In certain embodiments, the copolymer can include a block polymer.

In certain embodiments, the copolymer is a polyvinylimidazole-based copolymer. In certain embodiments, the polyvinylimidazole-based copolymer can be a copolymer of vinylimidazole and styrene or a derivative thereof.

In certain embodiments, the polyvinylimidazole-based copolymer can be a polyvinylimidazole-co-polystyrene polymer. In certain embodiments, the polyvinylimidazole-co-polystyrene polymer can be a poly(N-vinylimidazole)-co-polystyrene polymer, a poly(l-vinylimidazole)-co-polystyrene polymer, or a derivative thereof.

In certain embodiments, the copolymer is a polyvinylpyridine-based copolymer. In certain embodiments, the polyvinylpyridine-based copolymer can be a copolymer of vinylpyridine and styrene or a derivative thereof.

In certain embodiments, the polyvinylpyridine-based copolymer can be a polyvinylpyridine-co-polystyrene polymer. In certain embodiments, the polyvinylpyridine-co-polystyrene polymer can be a poly(4-vinylpyridine)-co-polystyrene polymer, a poly(2-vinylpyridine)-co-polystyrene polymer, or a derivative thereof.

In certain embodiments, the polyvinylpyridine-co-polystyrene polymer can include 1-50 mer % of styrene units. In certain embodiments, the polyvinylpyridine-co-polystyrene polymer can include 1-40 mer % of styrene units. In certain embodiments, the polyvinylpyridine-co-polystyrene polymer can include 1-30 mer % of styrene units.

In certain embodiments, a weight average molecular weight of the copolymer is in a range of about 5 kD-1,000 kD.

In certain embodiments, the crosslinker can be a diglycidyl- or triglycidyl-functional epoxy.

In certain embodiments, the crosslinker can be selected from the group consisting of diglycidyl-PEG (200-1000), glycerol triglycidyl ether, and a combination thereof.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, mol % crosslinking of the copolymer can be in a range of about 0.1 mol %-50 mol %. In certain embodiments, mol % crosslinking of the copolymer can be in a range of about 1 mol %-50 mol %. In certain embodiments, mol % crosslinking of the copolymer can be in a range of about 0.1 mol %-30 mol %. In certain embodiments, mol % crosslinking of the copolymer can be in a range of about 1 mol %-30 mol %. In certain embodiments, mol % crosslinking of the copolymer can be in a range of about 0.1 mol %-10 mol %. In certain embodiments, mol % crosslinking of the copolymer can be in a range of about 1 mol %-10 mol %.

In certain embodiments, the therapeutic agent can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.

In certain embodiments, the therapeutic agent can be an anti-inflammatory agent. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. In certain embodiments, the anti-inflammatory agent is dexamethasone or a derivative or a salt form thereof. In certain embodiments, the derivative of dexamethasone is dexamethasone acetate. In certain embodiments, the derivative of dexamethasone is dexamethasone sodium phosphate.

In certain embodiments, the drug delivery composition can include an amount of the therapeutic agent in a range of 0.01 wt %-50 wt % based on the total weight of the copolymer. In certain embodiments, the drug delivery composition can include an amount of the therapeutic agent in a range of 0.01 wt %-40 wt % based on the total weight of the copolymer.

In certain embodiments, the drug delivery composition can include about 0.1 μg to about 200 μg of the therapeutic agent. In certain embodiments, the drug delivery composition can include about 0.1 μg to about 20 μg of the therapeutic agent. In certain embodiments, the drug delivery composition can include about 0.1 μg to about 10 μg of the therapeutic agent.

In certain embodiments, the crosslinker is bonded to the hydrophilic unit of the copolymer to form a charge.

In certain embodiments, the therapeutic agent is not covalently bound to the copolymer.

In certain embodiments, the therapeutic agent is covalently bound to the copolymer.

In certain embodiments, the drug delivery composition continuously releases the therapeutic agent at a set or predetermined drug delivery rate for a set or predetermined days such as for at least 30 days.

In certain embodiments, the analyte sensor is configured to detect glucose.

One or more aspects of embodiments of the present disclosure are directed toward a method of manufacturing a drug delivery composition. In certain embodiments, the method can include: (a) providing a copolymer including a plurality of copolymer chains, wherein each of the plurality of copolymer chains comprises a backbone including a plurality of hydrophilic units and a plurality of hydrophobic units: (b) applying a crosslinker and a therapeutic agent to the copolymer: and (c) crosslinking the crosslinker to at least a portion of the hydrophilic units between respective copolymer chains.

The present disclosure further provides an analyte sensor as described herein for controlling a drug delivery rate of an analyte sensor, wherein the analyte sensor is implanted subcutaneously.

In certain embodiments, a drug delivery composition of the present disclosure can be used to control a drug delivery rate of an analyte sensor, wherein the drug delivery composition and the analyte sensor are inserted into the tissue of the subject. In certain embodiments, the drug delivery composition and the analyte sensor are inserted into the tissue of the subject using a sharp comprising the drug delivery composition and the analyte sensor. In certain embodiments, the analyte sensor is positioned within a channel of the sharp and the drug delivery composition is positioned distally to the analyte sensor within the channel of the sharp.

Additional aspects and embodiments will be set forth in part in the description which follows and, in part, will be apparent from the description, or can be learned by practice of the presented embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings are included to illustrate certain aspects of the present disclosure and should not be viewed as exclusive embodiments. The subject matter disclosed is capable of considerable modifications, alterations, combinations, and equivalents in form and function, without departing from the scope of the present disclosure.

FIG. 1 illustrates an exemplary drug delivery composition according to certain embodiments of the present disclosure.

FIGS. 2A-2C provide perspective view of exemplary analyte sensors including two active areas upon separate working electrodes.

FIG. 3 illustrates a cross-sectional diagram of an exemplary analyte sensor according to certain embodiments of the present disclosure.

FIG. 4 illustrates a cross-sectional diagram of an exemplary sharp according to certain embodiments of the present disclosure.

FIG. 5 illustrates exemplary coupons of drug delivery compositions on biocompatible strips according to certain embodiments of the present disclosure.

FIG. 6 illustrates exemplary sensor tails including drug delivery compositions according to certain embodiments of the present disclosure.

FIGS. 7A-7B illustrate exemplary test samples of a drug delivery composition according to certain embodiments of the present disclosure.

FIG. 8 illustrates an exemplary test procedure of a drug delivery composition according to certain embodiments of the present disclosure.

FIG. 9 illustrates an HPLC of dexamethasone according to certain embodiments of the present disclosure.

FIG. 10 illustrates a calibration curve of dexamethasone according to certain embodiments of the present disclosure.

FIG. 11 illustrates drug delivery profiles of exemplary drug delivery compositions including 100% polyvinylpyridine, glycerol triglycidyl ether (Gly3), and dexamethasone according to certain embodiments of the present disclosure.

FIG. 12 illustrates drug delivery profiles of exemplary drug delivery compositions including 100% polyvinylpyridine, diglycidyl-PEG 400 (PEG400), and dexamethasone according to certain embodiments of the present disclosure.

FIG. 13 illustrates exemplary tested polymers and copolymers of drug delivery compositions according to certain embodiments of the present disclosure.

FIG. 14 illustrates exemplary crosslinkers of drug delivery compositions according to certain embodiments of the present disclosure.

FIG. 15 illustrates exemplary formulations of drug delivery compositions according to certain embodiments of the present disclosure.

FIG. 16 illustrates drug delivery profiles of exemplary drug delivery compositions according to certain embodiments of the present disclosure.

FIG. 17 illustrates drug delivery profiles of exemplary drug delivery composition according to certain embodiments of the present disclosure.

FIG. 18 illustrates exemplary formulations of drug delivery compositions according to certain embodiments of the present disclosure.

FIG. 19 illustrates drug delivery profiles of exemplary drug delivery compositions according to certain embodiments of the present disclosure.

FIG. 20 illustrates drug delivery profiles of exemplary drug delivery composition according to certain embodiments of the present disclosure.

FIG. 21 illustrates drug delivery profiles of exemplary drug delivery compositions according to certain embodiments of the present disclosure.

FIG. 22 illustrates drug delivery profiles of exemplary drug delivery composition according to certain embodiments of the present disclosure.

FIG. 23 illustrates concentration relationships between different crosslinkers in exemplary drug delivery compositions according to certain embodiments of the present disclosure.

FIG. 24 illustrates drug delivery profiles of exemplary drug delivery compositions according to certain embodiments of the present disclosure.

FIG. 25 illustrates example formulations of exemplary drug delivery compositions for an analyte sensor according to certain embodiments of the present disclosure.

FIG. 26 illustrates drug delivery profiles of exemplary drug delivery compositions on an analyte sensor according to certain embodiments of the present disclosure.

FIG. 27 illustrates drug delivery profiles per timepoint of exemplary drug delivery compositions on an analyte sensor according to certain embodiments of the present disclosure.

FIG. 28 illustrates impact factors on the drug delivery rate of exemplary drug delivery compositions according to certain embodiments of the present disclosure.

FIG. 29 illustrates drug delivery profiles of exemplary drug delivery compositions according to certain embodiments of the present disclosure.

FIG. 30 illustrates drug delivery profiles of exemplary drug delivery compositions according to certain embodiments of the present disclosure.

FIG. 31 illustrates drug delivery profiles of exemplary drug delivery compositions according to certain embodiments of the present disclosure.

FIG. 32 illustrates drug delivery profiles and drug delivery profiles per timepoint of exemplary drug delivery compositions on an analyte sensor according to certain embodiments of the present disclosure.

FIG. 33 illustrates the Dex solubility in the polyvinylpyridine-ethanol:water (95:5 by volume) solution according to certain embodiments of the present disclosure.

FIG. 34A illustrates exemplary formulations of drug delivery compositions according to certain embodiments of the present disclosure.

FIG. 34B illustrates exemplary formulations of drug delivery compositions according to certain embodiments of the present disclosure.

FIG. 35A illustrates drug delivery profiles and drug delivery profiles per timepoint of exemplary drug delivery compositions on an analyte sensor according to certain embodiments of the present disclosure.

FIG. 35B illustrates drug delivery profiles and drug delivery profiles per timepoint of exemplary drug delivery compositions on an analyte sensor according to certain embodiments of the present disclosure.

FIG. 35C illustrates exemplary drug delivery loading amounts on an analyte sensor according to certain embodiments of the present disclosure and the effect of such amounts on LSA.

FIG. 36 is a flow chart illustrating a method of manufacturing an exemplary drug delivery composition according to certain embodiments of the present disclosure.

FIG. 37 provides a diagram of an illustrative sensing system that can incorporate an analyte sensor of the present disclosure.

FIGS. 38A-38B provide cross-sectional diagrams of exemplary analyte sensors including a single active area.

FIGS. 39A-39C provide cross-sectional diagrams of exemplary analyte sensors including two active areas upon separate working electrodes.

FIG. 40 provides a cross-sectional diagram of exemplary analyte sensors including two active areas.

### DETAILED DESCRIPTION

As described herein, the implantation of an analyte sensor can result in several physiological responses that can negatively impact sensor function. For example, inflammation or immune responses at sites of tissue trauma induced by the analyte sensor and its implantation can result in a loss of sensor functionality and sensitivity in vivo.

To address the foregoing needs, the present disclosure provides drug delivery compositions for treating tissue surrounding the implanted analyte sensor. For example, but not by way of limitation, the present disclosure provides analyte sensors that include a therapeutic agent, e.g., a drug delivery composition containing a therapeutic agent as described herein. In certain embodiments, the present disclosure provides drug delivery compositions that can be inserted near an analyte sensor implanted in a subject.

In certain embodiments, drug delivery compositions of the present disclosure provide sustained release of a therapeutic agent over an extended period of time, e.g., over a period of 14 days or longer, e.g., over a period of about 30 days. In certain embodiments, the sustained release of a therapeutic agent, e.g., an anti-inflammatory agent, in close proximity to an analyte sensor can result in the prevention and/or reduction of inflammation or immune responses in the tissue surrounding the implantation site. For example, but not by way of limitation, the prevention and/or reduction of inflammation in the tissue surrounding the implantation site can increase the life span of the implanted analyte sensor. In certain embodiments, preventing and/or reducing the immune response to the analyte sensor can increase the life span of the implanted analyte sensor. In certain embodiments, increasing the life span of the implanted analyte sensor refers to maintaining the accuracy of the analyte sensor towards the end of the life of the sensor and/or minimizing, reducing and/or eliminating analyte signal inaccuracies towards the end of the life of the sensor.

In certain embodiments, the life span of an analyte sensor disclosed herein can be increased by more than about 2 days, by more than about 3 days, by more than about 4 days, by more than about 5 days, by more than about 6 days, by more than about 7 days, by more than about 8 days, by more than about 9 days, by more than about 10 days, by more than about 11 days, by more than about 12 days, by more than about 13 days, by more than about 14 days, by more than about 15 days, by more than about 16 days, by more than about 17 days, by more than about 18 days, by more than about 19 days or by more than about 20 days. In certain embodiments, an analyte sensor that includes a drug delivery composition of the present disclosure can have a life span of about 14 days or more, about 15 days or more, about 16 days or more, about 17 days or more, about 18 days or more, about 19 days or more, about 20 days or more, about 21 days or more, about 22 days or more, about 23 days or more, about 24 days or more, about 25 days or more, about 26 days or more, about 27 days or more, about 28 days or more, about 29 days or more or about 30 days or more. In certain embodiments, the life span of an analyte sensor disclosed herein can be increased to obtain an analyte sensor that has a life span of about 30 days or greater.

Hereinafter, specific embodiments will be described in more detail so that those of ordinary skill in the art can easily implement them. For example, embodiments of the present disclosure will be explained in more detail referring to the drawings. However, the present disclosure can be embodied in many different forms and is not construed as limited to the example embodiments set forth herein.

For clarity, but not by way of limitation, the detailed description of the presently disclosed subject matter is divided into the following subsections:
I. Definitions;
II. Therapeutic Agents;
III. Drug Delivery Compositions;
IV. Analyte Sensors;
V. Delivery Devices and Methods of Delivery; and
VI. Exemplary Embodiments.

### I. Definitions

The terms utilized in the present disclosure generally have their ordinary meanings in the art, within the context of this disclosure and in the specific context where each term is utilized. Certain terms are discussed, or elsewhere in the specification, to provide additional guidance to the skilled artisan in describing the compositions and methods of the present disclosure and how to make and utilize them.

The terminology utilized herein is utilized to describe embodiments only and is not intended to limit the present disclosure. The singular expression includes the plural expression unless the context clearly dictates otherwise.

As utilized herein, the singular forms “a,” “an,” and “the” are intended to include the plural forms as well, unless the context clearly indicates otherwise. Further, the utilization of “may” when describing embodiments of the present disclosure refers to “one or more embodiments of the present disclosure.”

The terms “comprise(s),” “include(s),” “having,” “have/has,” “contain(s),” and variants thereof, as utilized herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

Herein, “or” is not to be construed as an exclusive meaning, for example, “A or B” is construed to include A, B, A+B, and/or the like. Further, as utilized herein, the term “and/or” includes any and all combinations of one or more of the associated listed items. Expressions such as “at least one of,” “one of,” and “selected from,” when preceding a list of elements, modify the entire list of elements and do not modify the individual elements of the list.

The term “about” or “approximately” refers to within an acceptable error range for the particular value as determined by one of ordinary skill in the art, which depends in part on how the value is measured or determined, i.e., the limitations of the measurement system. For example, “about” can refer to within 3 or more than 3 standard deviations, per the practice in the art. In certain embodiments, “about” may refer to a range of up to 20%, preferably up to 10%, more preferably up to 5%, and more preferably still up to 1% of a given value. In certain embodiments, particularly with respect to biological systems or processes, the term may refer to within an order of magnitude, preferably within 5-fold, and more preferably within 2-fold, of a value.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

As utilized herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glutamate, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, etc.

The term “biological fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears or the like. In certain embodiments, the biological fluid is dermal fluid or interstitial fluid. In certain embodiments, the biological fluid is interstitial fluid.

The term “covalent bond,” as utilized herein, refers to a chemical bond that involves the sharing of electron pairs between atoms. Likewise, “covalently bound” refers to chemical binding in a way that involves the sharing of electron pairs between atoms.

The term “non-covalent,” or the similar, as utilized herein, refers to a chemical interaction that does not involve the sharing of electrons, but rather involves more dispersed variations of electromagnetic interactions between molecules or within a molecule.

As utilized herein, the term “polyvinylpyridine-based polymer” refers to a polymer (e.g., a copolymer) that includes polyvinylpyridine (e.g., poly(2-vinylpyridine) or poly(4-vinylpyridine)) or a derivative thereof.

As used herein, the term “multi-component membrane” refers to a membrane comprising two or more types of membrane polymers.

As used herein, the term “single-component membrane” refers to a membrane comprising one type of membrane polymer.

The term “reference electrode” as used herein, can refer to either reference electrodes or electrodes that function as both, a reference and a counter electrode. Similarly, the term “counter electrode,” as used herein, refers to both, a counter electrode and a counter electrode that also functions as a reference electrode. In certain embodiments, the term “counter/reference electrode,” as used herein, refers to both, a counter electrode and a counter electrode that also functions as a reference electrode.

As utilized herein, the term “mol % crosslinking” can refer to a degree of crosslinking by a crosslinker in copolymer matrices of a drug delivery composition. For example, in certain embodiments, the copolymer can be a polyvinylpyridine-co-polystyrene copolymer, the “mol % crosslinking” of a crosslinker can be calculated by utilizing the following equation:

$$\frac{( mg Crosslinker ) \times ( 105 \frac{g}{mol} Pyridine ) \times Crosslinker Functionality}{( mg Polymer ) \times ( \frac{g}{mol} Crosslinker )} = mol \% crosslinking ,$$

where the crosslinker functionality is the number of reactive crosslinking groups in one crosslinker molecule.

### II. Therapeutic Agents

The present disclosure provides compositions of a therapeutic agent and analyte sensors that include a therapeutic agent. In certain embodiments, a composition (e.g., a drug delivery composition) or an analyte sensor of the present disclosure can include two or more therapeutic agents.

In certain embodiments, the therapeutic agent to be delivered according to the present disclosure can be a therapeutic agent that is effective at reducing, minimizing, preventing and/or inhibiting a tissue's response to analyte sensor implantation. For example, but not by way of limitation, the therapeutic agent to be delivered according to the present disclosure can be a therapeutic agent that is effective as reducing, minimizing, preventing and/or inhibiting inflammation in a tissue.

In certain embodiments, a therapeutic agent for use in the present disclosure can be an immunosuppressant. Non-limiting examples of immunosuppressants include anti-inflammatory agents, anti-cancer agents, anti-rejection drugs and combinations thereof.

In certain embodiments, a therapeutic agent for use in the present disclosure can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and combinations thereof. In certain embodiments, the therapeutic agent is an antibiotic agent. In certain embodiments, the therapeutic agent is an antiviral agent. In certain embodiments, the therapeutic agent is an anti-inflammatory agent. In certain embodiments, the therapeutic agent is an anti-cancer agent. In certain embodiments, the therapeutic agent is an antiplatelet agent. In certain embodiments, the therapeutic agent is an anticoagulant agent. In certain embodiments, the therapeutic agent is a coagulant agent. In certain embodiments, the therapeutic agent is an antiglycolytic agent.

In certain embodiments, the therapeutic agent is an antiviral agent. In certain embodiments, the antiviral agent can include, but is not limited to, Umifenovir, Baloxavir marboxil, Darunavir, Nitazoxanide, Peramivir, Tipranavir, etc.

In certain embodiments, the therapeutic agent is an antibiotic agent. In certain embodiments, the antibiotic agent can include, but is not limited to, Rifaximin, Ertapenem, Doripenem, Cefadroxil, Clindamycin, Amoxicillin, Penicillin, etc.

In certain embodiments, the therapeutic agent is an anti-cancer agent. In certain embodiments, the anti-cancer agent can include, but is not limited to, Gilteritinib, Glasdegib, Ivosidenib, Enasidenib, Midostaurin, Venetoclax, Alpelisib, etc.

In certain embodiments, the therapeutic agent can be an anti-inflammatory agent. In certain embodiments, the anti-inflammatory agent is a non-steroidal anti-inflammatory agent. In certain embodiments, the anti-inflammatory agent is a steroidal anti-inflammatory agent, e.g., a corticosteroid. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. Non-limiting salt forms include pharmaceutically acceptable salts including acetate and phosphate salts. In certain embodiments, the anti-inflammatory agent is a salt of dexamethasone.

In certain embodiments, the anti-inflammatory agent is dexamethasone or a derivative or a salt form thereof. In certain embodiments, the derivative and/or salt form of dexamethasone is dexamethasone acetate. In certain embodiments, the derivative and/or salt form of dexamethasone is dexamethasone sodium phosphate.

### III. Drug Delivery Compositions

The present disclosure provides compositions comprising one or more therapeutic agents and a polymer, e.g., drug delivery compositions. In certain embodiments, a drug delivery composition of the present disclosure can be incorporated into an analyte sensor, e.g., an implantable analyte sensor, described herein. In certain embodiments, a drug delivery composition of the present disclosure can be placed in close proximity to an analyte sensor, e.g., an implantable analyte sensor. The incorporation of a drug delivery composition within the analyte sensor itself or the delivery of a drug delivery composition in close proximity to the analyte sensor at its in vivo location allows for targeted delivery of the therapeutic agent contained within the drug delivery composition to the tissue surrounding the implantation site and the analyte sensor.

In certain embodiments, the targeted delivery of the therapeutic agent contained within the drug delivery composition to the site of analyte sensor implantation allows for the reduction of in vivo sensor failure due to FBR. In certain embodiments, the targeted delivery of the therapeutic agent contained within the drug delivery composition to the site of analyte sensor implantation allows for the reduction of sensor signal inaccuracies due to FBR. In certain embodiments, the targeted delivery of the therapeutic agent contained within the drug delivery composition to the site of analyte sensor implantation allows for the reduction in late sensor attenuation (LSA). For example, but not by way of limitation, the targeted delivery of the therapeutic agent to the site of analyte sensor implantation contained within the drug delivery composition allows for the reduction and/or elimination of analyte signal inaccuracies that can be observed after in vivo implantation.

In certain embodiments, the therapeutic agent can be incorporated into a drug delivery composition. For example, but not way of limitation, the therapeutic agent can be non-covalently mixed with a copolymer of the composition, or the therapeutic agent can be covalently attached to a copolymer of the composition. In certain embodiments, the therapeutic agent can be covalently attached directly or via a linker to one or more polymer chains of the composition. In certain embodiments, the therapeutic agent can be covalently attached to one or more polymer chains of the polymer matrix via a hydrolyzable bond to allow delayed release of the therapeutic agent after insertion of the analyte sensor in vivo.

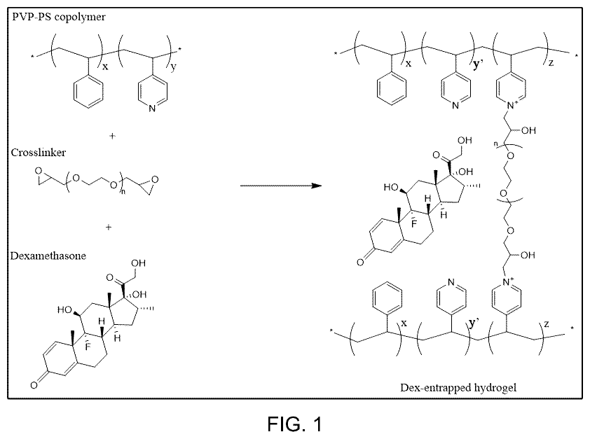

In certain embodiments, the therapeutic agent is non-covalently mixed with a copolymer of the composition, e.g., as shown in FIG. 1.

FIG. 1 illustrates an exemplary drug delivery composition according to some embodiments of the present disclosure. As shown in FIG. 1, certain embodiments of the present disclosure provide a drug delivery composition, and the drug delivery composition can include a polymer and a therapeutic agent. In certain embodiments, the drug delivery composition can include a copolymer including a plurality of copolymer chains and a therapeutic agent. In certain embodiments, each of the plurality of copolymer chains comprises a backbone including a plurality of hydrophilic units and a plurality of hydrophobic units. In certain embodiments, a drug delivery composition of the present disclosure further comprises a crosslinker, e.g., that crosslinks at least a portion of the hydrophilic units between respective copolymer chains. For example, but not by way of limitation, the drug delivery composition can include (i) a copolymer including a plurality of copolymer chains, where each of the plurality of copolymer chains comprises a backbone comprising a plurality of hydrophilic units and a plurality of hydrophobic units, (ii) a crosslinker crosslinking at least a portion of the hydrophilic units between respective copolymer chains, and (iii) a therapeutic agent, as shown in FIG. 1.

In certain embodiments, the hydrophilic unit of the copolymer can include a nitrogen-containing heterocyclic unit. Non-limiting examples of a nitrogen-containing heterocyclic unit include a pyridine unit, a pyridazine unit, a pyrimidine unit, a pyrazine unit, a triazine unit, an imidazole unit, a pyrazole unit, etc. For example, in certain embodiments, the hydrophilic unit of the copolymer, as shown in FIG. 1, can be a pyridine unit. Of course, embodiments of the present disclosure are not limited thereto.

In certain embodiments, the hydrophobic unit of the copolymer can include a non-heteroatom containing aromatic unit such as a benzene (phenyl) unit, a naphthalene unit, an anthracene unit, etc., an acyclic aliphatic unit such as a straight or branched alkyl unit, a straight or branched alkenyl unit, a straight or branched alkynyl unit, etc., and/or a cyclic aliphatic unit such as a cyclobutyl, a cyclopentyl unit, a cyclohexyl unit, a cycloheptyl unit, a cyclooctyl unit, a cyclohexenyl unit, etc. For example, in certain embodiments, the hydrophobic unit of the copolymer, as shown in FIG. 1, can be a benzene (phenyl) unit. Of course, embodiments of the present disclosure are not limited thereto.

In certain embodiments, the copolymer can be an alternating copolymer, a random copolymer, a block copolymer, or a graft copolymer. In certain embodiments, the copolymer can be a graft copolymer. In certain embodiments, the copolymer can be an alternating copolymer. In certain embodiments, the copolymer can be a random copolymer. In certain embodiments, the copolymer can be a block copolymer.

In certain embodiments, a mer % of the hydrophobic unit, i.e., a ratio of x/(x+y) shown in FIG. 1, in the copolymer is in a range of about 1%-99%, about 1%-90%, about 1%-80%, about 1%-70%, about 1%-60%, about 1%-50%, about 1%-40%, about 1%-30%, about 1%-25%, about 1%-20%, about 1%-15%, about 1%-10%, about 2%-10%, about 3%-10%, about 4%-10%, about 5%-10%, about 6%-10%, about 7%-10%, about 8%-10%, or about 9%-10%, or with any range defined between any two of the foregoing values, such as in a range of about 7%-15%. In certain embodiments, the mer % of the hydrophobic unit in the copolymer is in a range of about 5%-25%. In certain embodiments, the mer % of the hydrophobic unit in the copolymer is in a range of about 5%-20%. In certain embodiments, the mer % of the hydrophobic unit in the copolymer is in a range of about 5%-15%. In certain embodiments, the mer % of the hydrophobic unit in the copolymer is in a range of about 5%-10%.

In certain embodiments, the copolymer can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.

In certain embodiments, the copolymer can be a polyurethane-based copolymer. Non-limiting examples of a polyurethane-based copolymer includes an ether-based polyurethane or an ester-based polyurethane.

In certain embodiments, the copolymer can be a polyvinylimidazole-based copolymer. In certain embodiments, the polyvinylimidazole-based copolymer can be a copolymer of vinylimidazole and styrene or a derivative thereof.

In certain embodiments, the polyvinylimidazole-based copolymer can be a polyvinylimidazole-co-polystyrene polymer. In certain embodiments, the polyvinylimidazole-co-polystyrene polymer can be a poly(N-vinylimidazole)-co-polystyrene polymer, a poly(1-vinylimidazole)-co-polystyrene polymer, or a derivative thereof.

In certain embodiments, the copolymer can be a polyvinylpyridine-based copolymer. In certain embodiments, the polyvinylpyridine-based copolymer can be a copolymer of vinylpyridine and styrene or a derivative thereof.

In certain embodiments, the polyvinylpyridine-based copolymer can be a polyvinylpyridine-co-polystyrene polymer. In certain embodiments, the polyvinylpyridine-co-polystyrene polymer comprises poly(4-vinylpyridine) and styrene. In certain embodiments, the polyvinylpyridine-co-polystyrene polymer comprises poly(2-vinylpyridine) and styrene. In certain embodiments, the polyvinylpyridine-co-polystyrene polymer can be a poly(4-vinylpyridine)-co-polystyrene polymer, a poly(2-vinylpyridine)-co-polystyrene polymer, or a derivative thereof.

In certain embodiments, the polyvinylpyridine-co-polystyrene polymer can include about 1-50 mer % of styrene units, about 1-40 mer % of styrene units, about 1-30 mer % of styrene units, about 1-20 mer % of styrene units, or about 1-15 mer % of styrene units. In certain embodiments, the polyvinylpyridine-co-polystyrene polymer can include about 1-50 mer % of styrene units. In certain embodiments, the polyvinylpyridine-co-polystyrene polymer can include about 1-40 mer % of styrene units. In certain embodiments, the polyvinylpyridine-co-polystyrene polymer can include about 1-30 mer % of styrene units. In certain embodiments, the polyvinylpyridine-co-polystyrene polymer can include about 1-20 mer % of styrene units. In certain embodiments, the polyvinylpyridine-co-polystyrene polymer can include about 1-15 mer % of styrene units.

In certain embodiments, the polyvinylpyridine-co-polystyrene polymer can include about 5-25 mer % of styrene units. In certain embodiments, the polyvinylpyridine-co-polystyrene polymer can include about 5-20 mer % of styrene units. In certain embodiments, the polyvinylpyridine-co-polystyrene polymer can include about 5-15 mer % of styrene units.

In certain embodiments, a weight average molecular weight of the copolymer is in a range of about 5 kD-1,000 kD, about 5 kD-900 kD, about 5 kD-800 kD, about 5 kD-700 kD, about 5 kD-600 kD, about 5 kD-500 kD, about 5 kD-400 kD, about 5 kD-300 kD, about 10 kD-300 kD, about 20 kD-300 kD, about 30 kD-300 kD, about 40 kD-300 kD, about 50 kD-300 kD, about 60 kD-300 kD, about 70 kD-300 kD, about 80 kD-300 kD, about 90 kD-300 kD, about 100 kD-300 kD, or about 100 kD-200 kD, or with any range defined between any two of the foregoing values, such as in a range of about 100 kD-400 kD. In certain embodiments, a weight average molecular weight of the copolymer is in a range of about 100 kD-250 kD. In certain embodiments, the molecular weight of a copolymer can be determined by a suitable method such as gel permeation chromatography.

In certain embodiments, the copolymer for use in the present disclosure is capable of absorbing from about 5% to about 95% of its weight in water. For example, but not by way of limitation, the copolymer for use in the present disclosure is capable of absorbing from about 5% to about 95%, from about 5% to about 90%, from about 5% to about 85%, from about 10% to about 95%, from about 15% to about 95%, from about 20% to about 95%, from about 25% to about 95%, from about 30% to about 95%, from about 5% to about 30%, from about 5% to about 35%, from about 5% to about 25% or from about 5% to about 20%. In certain embodiments, the copolymer is capable of absorbing at least about 5% of its weight in water. In certain embodiments, the copolymer is capable of absorbing at least about 10% of its weight in water. In certain embodiments, the copolymer is capable of absorbing at least about 20% of its weight in water. In certain embodiments, the copolymer is capable of absorbing at least about 30% of its weight in water. In certain embodiments, the copolymer is capable of absorbing at least about 40% of its weight in water. In certain embodiments, the copolymer is capable of absorbing at least about 50% of its weight in water. In certain embodiments, the copolymer is capable of absorbing at least about 60% of its weight in water. In certain embodiments, the copolymer is capable of absorbing at least about 70% of its weight in water. In certain embodiments, the copolymer is capable of absorbing at least about 80% of its weight in water. In certain embodiments, the copolymer is capable of absorbing at least about 90% of its weight in water. In certain embodiments, the copolymer is capable of absorbing at least about 95% of its weight in water. In certain embodiments, the copolymer is capable of absorbing from about 5% to about 25% of its weight in water.

In certain embodiments, the copolymer for use in the present disclosure can have a hardness from about 20 to about 100 Shore A. For example, but not by way of limitation, the copolymer for use in the present disclosure can have a hardness from about 20 to about 90 Shore A, about 20 to about 80 Shore A, about 20 to about 70 Shore A, about 20 to about 60 Shore A, about 20 to about 50 Shore A, about 20 to about 40 Shore A, about 20 to about 30 Shore A, about 30 to about 100 Shore A, about 40 to about 100 Shore A, about 50 to about 100 Shore A, about 60 to about 100 Shore A, about 70 to about 100 Shore A, about 80 to about 100 Shore A, about 90 to about 100 Shore A, about 70 to about 95 Shore A, about 70 to about 90 Shore A, from about 70 to about 85 Shore A, from about 70 to about 80 Shore A, from about 75 to about 95 Shore A, from about 80 to about 95 Shore A, from about 85 to about 95 Shore A, from about 80 to about 93 Shore A or from about 80 to about 90 Shore A. In certain embodiments, the copolymer for use in the present disclosure can have a hardness of about 80 Shore A. In certain embodiments, the copolymer for use in the present disclosure can have a hardness of about 90 Shore A. In certain embodiments, the copolymer for use in the present disclosure can have a hardness of about 93 Shore A. In certain embodiments, the copolymer for use in the present disclosure can have a hardness of about 80 to about 100 Shore A, e.g., prior to implantation in a subject or prior to being hydrated. In certain embodiments, the copolymer for use in the present disclosure can have a hardness of about 20 to about 60 Shore A, e.g., when implanted in a subject or when hydrated.

In certain embodiments, the linear expansion of a copolymer for use in the present disclosure is from about 10% to about 200%. For example, but not by way of limitation, the linear expansion of a copolymer for use in the present disclosure can be from about 10% to about 190%, from about 10% to about 180%, from about 10% to about 170%, from about 10% to about 160%, from about 10% to about 150%, from about 10% to about 140%, from about 10% to about 130%, from about 10% to about 120%, from about 10% to about 110%, from about 10% to about 100%, from about 25% to about 100%, from about 30% to about 100%, from about 35% to about 100%, from about 40% to about 100%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 20% to about 95%, from about 20% to about 90%, from about 20% to about 85%, from about 20% to about 80%, from about 20% to about 75%, from about 20% to about 70%, from about 20% to about 65%, from about 20% to about 60%, from about 20% to about 55%, from about 20% to about 50%, from about 20% to about 45%, from about 20% to about 40%, from about 20% to about 30%, from about 30% to about 60%, from about 40% to about 50%, from about 40% to about 60%, from about 20% to about 30% or from about 50% to about 70%. In certain embodiments, the linear expansion of a copolymer for use in the present disclosure is about 25%. In certain embodiments, the linear expansion of a copolymer for use in the present disclosure is about 40%. In certain embodiments, the linear expansion of a copolymer for use in the present disclosure is about 45%. In certain embodiments, the linear expansion of a copolymer for use in the present disclosure is about 50%. In certain embodiments, the linear expansion of a copolymer for use in the present disclosure is about 60%. In certain embodiments, the linear expansion of a copolymer for use in the present disclosure is of about 100%. In certain embodiments, the linear expansion of a copolymer for use in the present disclosure is about 110% or greater, of about 100% or greater, of about 90% or greater, of about 80% or greater, of about 70% or greater, of about 60% or greater, of about 50% or greater, of about 40% or greater, of about 30% or greater, of about 20% or greater or of about 10% or greater. In certain embodiments, the linear expansion of a copolymer for use in the present disclosure is about 110% or greater. In certain embodiments, the linear expansion of a copolymer for use in the present disclosure is about 100% or greater. In certain embodiments, the linear expansion of a copolymer for use in the present disclosure is about 90% or greater. In certain embodiments, the linear expansion of a copolymer for use in the present disclosure is about 80% or greater. In certain embodiments, the linear expansion of a copolymer for use in the present disclosure is about 70% or greater. In certain embodiments, the linear expansion of a copolymer for use in the present disclosure is about 60% or greater. In certain embodiments, the linear expansion of a copolymer for use in the present disclosure is about 50% or greater. In certain embodiments, the linear expansion of a copolymer for use in the present disclosure is about 40% or greater. In certain embodiments, the linear expansion of a copolymer for use in the present disclosure is about 30% or greater. In certain embodiments, the linear expansion of a copolymer for use in the present disclosure is about 20% or greater. In certain embodiments, the linear expansion of a copolymer for use in the present disclosure is about 10% or greater.

In certain embodiments, the copolymer has a linear expansion of about 45% and is capable of absorbing about 70% of its weight in water. In certain embodiments, the copolymer has a linear expansion of about 25% and is capable of absorbing about 55% of its weight in water. In certain embodiments, the copolymer has a linear expansion of about 40% and is capable of absorbing about 60% of its weight in water. In certain embodiments, the copolymer has a linear expansion of about 50% and is capable of absorbing about 50% of its weight in water. In certain embodiments, the copolymer has a linear expansion of about 60% and is capable of absorbing about 80% of its weight in water. In certain embodiments, the copolymer has a linear expansion of about 100% and is capable of absorbing about 90% of its weight in water. In certain embodiments, the copolymer has a linear expansion of about 10% and is capable of absorbing about 30% of its weight in water. In certain embodiments, the copolymer has a linear expansion of about 180% and is capable of absorbing about 95% of its weight in water.

In certain embodiments, a drug delivery composition of the present disclosure can further include a crosslinker. For example, but not by way of limitation, the crosslinker crosslinks at least a portion of the hydrophilic and/or hydrophobic units between respective copolymer chains. In certain embodiments, the crosslinker crosslinks at least a portion of the hydrophilic units between respective copolymer chains. In certain embodiments, the crosslinker crosslinks at least a portion of the nitrogen-containing heterocyclic units, e.g., pyridine units, between respective copolymer chains. For example, but not by way of limitation, the crosslinker crosslinks at least a portion of the pyridine units between respective copolymer chains as shown in FIG. 1.

In certain embodiments, the crosslinker can be a diglycidyl- or triglycidyl-functional epoxy.

In certain embodiments, the crosslinker can be selected from the group consisting of diglycidyl-PEG (200-1000), glycerol triglycidyl ether, and a combination thereof. For example, in certain embodiments, as shown in FIG. 1, the crosslinker is a diglycidyl-PEG (200-1000) with a molecular weight of 200 g/mol-1000 g/mol. The term “diglycidyl-PEG” utilized in the present disclosure can refer to polyethylene glycol diglycidyl ether.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, a drug delivery composition comprises a crosslinker (e.g., includes a certain amount of crosslinker) that provides a certain mol % of crosslinking of the polymer, e.g., the copolymer, present within the drug delivery composition. In certain embodiments, mol % crosslinking of the copolymer can be in a range of about 0.1 mol %-50 mol %. In certain embodiments, mol % crosslinking of the copolymer can be in a range of about 1 mol %-50 mol %. In certain embodiments, mol % crosslinking of the copolymer can be in a range of about 0.1 mol %-40 mol %. In certain embodiments, mol % crosslinking of the copolymer can be in a range of about 1 mol %-40 mol %. In certain embodiments, mol % crosslinking of the copolymer can be in a range of about 0.1 mol %-30 mol %. In certain embodiments, mol % crosslinking of the copolymer can be in a range of about 0.2 mol %-30 mol %. In certain embodiments, mol % crosslinking of the copolymer can be in a range of about 1 mol %-30 mol %. In certain embodiments, mol % crosslinking of the copolymer can be in a range of about 0.1 mol %-25 mol %. In certain embodiments, mol % crosslinking of the copolymer can be in a range of about 1 mol %-25 mol %. In certain embodiments, mol % crosslinking of the copolymer can be in a range of about 0.1 mol %-20 mol %. In certain embodiments, mol % crosslinking of the copolymer can be in a range of about 1 mol %-20 mol %. In certain embodiments, mol % crosslinking of the copolymer can be in a range of about 0.1 mol %-15 mol %. In certain embodiments, mol % crosslinking of the copolymer can be in a range of about 1 mol %-15 mol %. In certain embodiments, mol % crosslinking of the copolymer can be in a range of about 0.1 mol %-10 mol %. In certain embodiments, mol % crosslinking of the copolymer can be in a range of about 0.5 mol %-10 mol %. In certain embodiments, mol % crosslinking of the copolymer can be in a range of about 1 mol %-10 mol %.

In certain embodiments, a drug delivery composition of the present disclosure includes a crosslinker, where the amount of crosslinker in the drug delivery composition provides no more than about 20 mol % crosslinking (e.g., of the copolymer). In certain embodiments, a drug delivery composition of the present disclosure includes a crosslinker, where the amount of crosslinker in the drug delivery composition provides no more than about 19 mol % crosslinking (e.g., of the copolymer). In certain embodiments, a drug delivery composition of the present disclosure includes a crosslinker, where the amount of crosslinker in the drug delivery composition provides no more than about 18 mol % crosslinking (e.g., of the copolymer). In certain embodiments, a drug delivery composition of the present disclosure includes a crosslinker, where the amount of crosslinker in the drug delivery composition provides no more than about 17 mol % crosslinking (e.g., of the copolymer). In certain embodiments, a drug delivery composition of the present disclosure includes a crosslinker, where the amount of crosslinker in the drug delivery composition provides no more than about 16 mol % crosslinking (e.g., of the copolymer). In certain embodiments, a drug delivery composition of the present disclosure includes a crosslinker, where the amount of crosslinker in the drug delivery composition provides no more than about 15 mol % crosslinking (e.g., of the copolymer). In certain embodiments, a drug delivery composition of the present disclosure includes a crosslinker, where the amount of crosslinker in the drug delivery composition provides no more than about 14 mol % crosslinking (e.g., of the copolymer). In certain embodiments, a drug delivery composition of the present disclosure includes a crosslinker, where the amount of crosslinker in the drug delivery composition provides no more than about 13 mol % crosslinking (e.g., of the copolymer). In certain embodiments, a drug delivery composition of the present disclosure includes a crosslinker, where the amount of crosslinker in the drug delivery composition provides no more than about 12 mol % crosslinking (e.g., of the copolymer). In certain embodiments, a drug delivery composition of the present disclosure includes a crosslinker, where the amount of crosslinker in the drug delivery composition provides no more than about 11 mol % crosslinking (e.g., of the copolymer). In certain embodiments, a drug delivery composition of the present disclosure includes a crosslinker, where the amount of crosslinker in the drug delivery composition provides no more than about 10 mol % crosslinking (e.g., of the copolymer). In certain embodiments, a drug delivery composition of the present disclosure includes a crosslinker, where the amount of crosslinker in the drug delivery composition provides no more than about 9 mol % crosslinking (e.g., of the copolymer). In certain embodiments, a drug delivery composition of the present disclosure includes a crosslinker, where the amount of crosslinker in the drug delivery composition provides no more than about 8 mol % crosslinking (e.g., of the copolymer). In certain embodiments, a drug delivery composition of the present disclosure includes a crosslinker, where the amount of crosslinker in the drug delivery composition provides no more than about 7 mol % crosslinking (e.g., of the copolymer). In certain embodiments, a drug delivery composition of the present disclosure includes a crosslinker, where the amount of crosslinker in the drug delivery composition provides no more than about 6 mol % crosslinking (e.g., of the copolymer). In certain embodiments, a drug delivery composition of the present disclosure includes a crosslinker, where the amount of crosslinker in the drug delivery composition provides no more than about 5 mol % crosslinking (e.g., of the copolymer).

In certain embodiments, the drug delivery composition can include an amount of the crosslinker in a range of about 0.01 wt %-50 wt % based on the total weight of the copolymer (e.g., in the drug delivery composition). In certain embodiments, the drug delivery composition can include an amount of the crosslinker in a range of about 0.01 wt %-40 wt % based on the total weight of the copolymer. In certain embodiments, the drug delivery composition can include an amount of the crosslinker in a range of about 0.01 wt %-30 wt % based on the total weight of the copolymer. In certain embodiments, the drug delivery composition can include an amount of the crosslinker in a range of about 0.01 wt %-20 wt % based on the total weight of the copolymer. In certain embodiments, the drug delivery composition can include an amount of the crosslinker in a range of about 0.01 wt %-15 wt % based on the total weight of the copolymer. In certain embodiments, the drug delivery composition can include an amount of the crosslinker in a range of about 0.01 wt %-10 wt % based on the total weight of the copolymer. In certain embodiments, the drug delivery composition can include an amount of the crosslinker in a range of about 0.01 wt %-9 wt % based on the total weight of the copolymer. In certain embodiments, the drug delivery composition can include an amount of the crosslinker in a range of about 0.01 wt %-8 wt % based on the total weight of the copolymer. In certain embodiments, the drug delivery composition can include an amount of the crosslinker in a range of about 0.01 wt %-7 wt % based on the total weight of the copolymer. In certain embodiments, the drug delivery composition can include an amount of the crosslinker in a range of about 0.01 wt %-6 wt % based on the total weight of the copolymer. In certain embodiments, the drug delivery composition can include an amount of the crosslinker in a range of about 0.01 wt %-5 wt % based on the total weight of the copolymer. In certain embodiments, the drug delivery composition can include an amount of the crosslinker in a range of about 0.01 wt %-4 wt % based on the total weight of the copolymer. In certain embodiments, the drug delivery composition can include an amount of the crosslinker in a range of about 0.01 wt %-3 wt % based on the total weight of the copolymer. In certain embodiments, the drug delivery composition can include an amount of the crosslinker in a range of about 0.01 wt %-2 wt % based on the total weight of the copolymer. In certain embodiments, the drug delivery composition can include an amount of the crosslinker in a range of about 0.01 wt %-1 wt % based on the total weight of the copolymer. In certain embodiments, the drug delivery composition can include an amount of the crosslinker in a range of about 0.01 wt %-5 wt % based on the total weight of the copolymer. In certain embodiments, the drug delivery composition can include an amount of the crosslinker in a range of about 1 wt %-40 wt % based on the total weight of the copolymer. In certain embodiments, the drug delivery composition can include an amount of the crosslinker in a range of about 1 wt %-30 wt % based on the total weight of the copolymer. In certain embodiments, the drug delivery composition can include an amount of the crosslinker in a range of about 1 wt %-25 wt % based on the total weight of the copolymer. In certain embodiments, the drug delivery composition can include an amount of the crosslinker in a range of about 1 wt %-20 wt % based on the total weight of the copolymer. In certain embodiments, the drug delivery composition can include an amount of the crosslinker in a range of about 1 wt %-15 wt % based on the total weight of the copolymer. In certain embodiments, the drug delivery composition can include an amount of the crosslinker in a range of about 1 wt %-10 wt % based on the total weight of the copolymer. In certain embodiments, the drug delivery composition can include an amount of the crosslinker in a range of about 1 wt %-5 wt % based on the total weight of the copolymer.

As shown in FIG. 1, the crosslinker crosslinks at least two polymer backbones of two or more copolymer chains through bonding to the hydrophilic units of the two or more copolymer chains. In certain embodiments, the copolymer can be a polyvinylpyridine-co-polystyrene polymer, and the crosslinker can be diglycidyl-PEG. The diglycidyl-PEG can have two crosslinking glycidyl groups, each of them can bond to the nitrogen atom of pyridine of different copolymer chains to crosslink the backbone of the copolymer chains, and positive charges are formed on the pyridine moieties, as shown in FIG. 1. The formed charges can regulate the swellability of the drug delivery composition. Furthermore, by utilizing a suitable amount of diglycidyl-PEG with a suitable molecular weight and/or glycerol triglycidyl ether which has three crosslinkable glycidyl groups, the degree of crosslinking of the copolymer can be finely tuned as shown in the examples.

In certain embodiments, the drug delivery composition can include one or more therapeutic agents. Non-limiting examples of therapeutic agents are disclosed herein in Section II. For example, but not by way of limitation, the therapeutic agent can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof. In certain embodiments, the therapeutic agent is an anti-inflammatory agent. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. In certain embodiments, the anti-inflammatory agent is dexamethasone or a derivative or a salt form thereof. In certain embodiments, the derivative and/or salt form of dexamethasone is dexamethasone acetate. In certain embodiments, the derivative and/or salt form of dexamethasone is dexamethasone sodium phosphate.

As shown in FIG. 1, in certain embodiments, the therapeutic agent is dexamethasone. Dexamethasone is non-covalently present in the crosslinked copolymer matrices and trapped in the crosslinked copolymer matrices. Dexamethasone can interact with the hydrophilic units and hydrophobic units of the crosslinked copolymer matrices through non-polar and polar interactions.

In certain embodiments, the therapeutic agent can be covalently bound to the copolymer.

In certain embodiments, the drug delivery composition can include an amount of the therapeutic agent, e.g., dexamethasone or a derivative thereof, in a range of about 0.01 wt %-50 wt % based on the total weight of the copolymer. In certain embodiments, the drug delivery composition can include an amount of the therapeutic agent, e.g., dexamethasone or a derivative thereof, in a range of about 0.01 wt %-40 wt % based on the total weight of the copolymer. In certain embodiments, the drug delivery composition can include an amount of the therapeutic agent, e.g., dexamethasone or a derivative thereof, in a range of about 1 wt %-40 wt % based on the total weight of the copolymer. In certain embodiments, the drug delivery composition can include an amount of the therapeutic agent, e.g., dexamethasone or a derivative thereof, in a range of about 5 wt %-40 wt % based on the total weight of the copolymer. In certain embodiments, the drug delivery composition can include an amount of the therapeutic agent, e.g., dexamethasone or a derivative thereof, in a range of 10 wt %-40 wt % based on the total weight of the copolymer. In certain embodiments, the drug delivery composition can include an amount of the therapeutic agent, e.g., dexamethasone or a derivative thereof, in a range of about 20 wt %-40 wt % based on the total weight of the copolymer. In certain embodiments, the drug delivery composition can include an amount of the therapeutic agent, e.g., dexamethasone or a derivative thereof, in a range of about 30 wt %-40 wt % based on the total weight of the copolymer. In certain embodiments, the drug delivery composition can include an amount of the therapeutic agent, e.g., dexamethasone or a derivative thereof, in a range of about 5 wt %-20 wt % based on the total weight of the copolymer. In certain embodiments, the drug delivery composition can include an amount of the therapeutic agent, e.g., dexamethasone or a derivative thereof, in a range of about 5 wt %-10 wt % based on the total weight of the copolymer. In certain embodiments, the drug delivery composition can include an amount of the therapeutic agent, e.g., dexamethasone or a derivative thereof, in a range of about 1 wt %-10 wt % based on the total weight of the copolymer. In certain embodiments, the drug delivery composition can include an amount of the therapeutic agent, e.g., dexamethasone or a derivative thereof, in a range of about 1 wt %-20 wt % based on the total weight of the copolymer. In certain embodiments, the drug delivery composition can include an amount of the therapeutic agent, e.g., dexamethasone or a derivative thereof, in a range of about 1 wt %-30 wt % based on the total weight of the copolymer. In certain embodiments, the drug delivery composition can include an amount of the therapeutic agent, e.g., dexamethasone or a derivative thereof, in a range of about 10 wt %-20 wt % based on the total weight of the copolymer.

In certain embodiments, the drug delivery composition can include an amount of the therapeutic agent, e.g., dexamethasone or a derivative thereof, that is no greater than about 50 wt % based on the total weight of the copolymer. In certain embodiments, the drug delivery composition can include an amount of the therapeutic agent, e.g., dexamethasone or a derivative thereof, that is no greater than about 49 wt % based on the total weight of the copolymer. In certain embodiments, the drug delivery composition can include an amount of the therapeutic agent, e.g., dexamethasone or a derivative thereof, that is no greater than about 48 wt % based on the total weight of the copolymer. In certain embodiments, the drug delivery composition can include an amount of the therapeutic agent, e.g., dexamethasone or a derivative thereof, that is no greater than about 47 wt % based on the total weight of the copolymer. In certain embodiments, the drug delivery composition can include an amount of the therapeutic agent, e.g., dexamethasone or a derivative thereof, that is no greater than about 46 wt % based on the total weight of the copolymer. In certain embodiments, the drug delivery composition can include an amount of the therapeutic agent, e.g., dexamethasone or a derivative thereof, that is no greater than about 45 wt % based on the total weight of the copolymer. In certain embodiments, the drug delivery composition can include an amount of the therapeutic agent, e.g., dexamethasone or a derivative thereof, that is no greater than about 44 wt % based on the total weight of the copolymer. In certain embodiments, the drug delivery composition can include an amount of the therapeutic agent, e.g., dexamethasone or a derivative thereof, that is no greater than about 43 wt % based on the total weight of the copolymer. In certain embodiments, the drug delivery composition can include an amount of the therapeutic agent, e.g., dexamethasone or a derivative thereof, that is no greater than about 42 wt % based on the total weight of the copolymer. In certain embodiments, the drug delivery composition can include an amount of the therapeutic agent, e.g., dexamethasone or a derivative thereof, that is no greater than about 41 wt % based on the total weight of the copolymer. In certain embodiments, the drug delivery composition can include an amount of the therapeutic agent, e.g., dexamethasone or a derivative thereof, that is no greater than about 40 wt % based on the total weight of the copolymer. In certain embodiments, the drug delivery composition can include an amount of the therapeutic agent, e.g., dexamethasone or a derivative thereof, that is no greater than about 39 wt % based on the total weight of the copolymer. In certain embodiments, the drug delivery composition can include an amount of the therapeutic agent, e.g., dexamethasone or a derivative thereof, that is no greater than about 38 wt % based on the total weight of the copolymer. In certain embodiments, the drug delivery composition can include an amount of the therapeutic agent, e.g., dexamethasone or a derivative thereof, that is no greater than about 37 wt % based on the total weight of the copolymer. In certain embodiments, the drug delivery composition can include an amount of the therapeutic agent, e.g., dexamethasone or a derivative thereof, that is no greater than about 36 wt % based on the total weight of the copolymer. In certain embodiments, the drug delivery composition can include an amount of the therapeutic agent, e.g., dexamethasone or a derivative thereof, that is no greater than about 35 wt % based on the total weight of the copolymer. In certain embodiments, the drug delivery composition can include an amount of the therapeutic agent, e.g., dexamethasone or a derivative thereof, that is no greater than about 34 wt % based on the total weight of the copolymer. In certain embodiments, the drug delivery composition can include an amount of the therapeutic agent, e.g., dexamethasone or a derivative thereof, that is no greater than about 33 wt % based on the total weight of the copolymer. In certain embodiments, the drug delivery composition can include an amount of the therapeutic agent, e.g., dexamethasone or a derivative thereof, that is no greater than about 32 wt % based on the total weight of the copolymer. In certain embodiments, the drug delivery composition can include an amount of the therapeutic agent, e.g., dexamethasone or a derivative thereof, that is no greater than about 31 wt % based on the total weight of the copolymer. In certain embodiments, the drug delivery composition can include an amount of the therapeutic agent, e.g., dexamethasone or a derivative thereof, that is no greater than about 30 wt % based on the total weight of the copolymer. In certain embodiments, the drug delivery composition can include an amount of the therapeutic agent, e.g., dexamethasone or a derivative thereof, that is no greater than about 25 wt % based on the total weight of the copolymer. In certain embodiments, the drug delivery composition can include an amount of the therapeutic agent, e.g., dexamethasone or a derivative thereof, that is no greater than about 20 wt % based on the total weight of the copolymer. In certain embodiments, the drug delivery composition can include an amount of the therapeutic agent, e.g., dexamethasone or a derivative thereof, that is no greater than about 15 wt % based on the total weight of the copolymer. In certain embodiments, the drug delivery composition can include an amount of the therapeutic agent, e.g., dexamethasone or a derivative thereof, that is no greater than about 10 wt % based on the total weight of the copolymer.

In certain embodiments, the drug delivery composition can include from about 0.0005 mg to about 0.2 mg of the therapeutic agent, e.g., dexamethasone, or any values in between. In certain embodiments, the drug delivery composition can include about 0.0005 mg, about 0.001 mg, about 0.005 mg, about 0.01 mg, about 0.05 mg, about 0.1 mg or about 0.2 mg of the therapeutic agent, e.g., dexamethasone. In certain embodiments, the drug delivery composition can include from about 0.0005 mg to about 0.1 mg, about 0.0005 mg to about 0.05 mg, about 0.0005 mg to about 0.01 mg, about 0.0005 mg to about 0.005 mg or about 0.0005 mg to about 0.001 mg of the therapeutic agent, e.g., dexamethasone. In certain embodiments, the drug delivery composition can include from about 0.0005 mg to about 0.1 mg. In certain embodiments, the drug delivery composition can include from about 0.0005 mg to about 0.01 mg. In certain embodiments, the drug delivery composition can include from about 0.001 mg to about 0.1 mg. In certain embodiments, the drug delivery composition can include from about 0.001 mg to about 0.01 mg. In certain embodiments, the drug delivery composition can include from about 0.001 mg to about 0.005 mg. In certain embodiments, the drug delivery composition can include from about 0.001 mg to about 0.003 mg.

In certain embodiments, the drug delivery composition can include from about 0.1 μg to about 200 μg of the therapeutic agent, e.g., from about 0.5 μg to about 200 μg, from about 1 μg to about 200 μg, from about 1.5 μg to about 200 μg, from about 2.0 μg to about 200 μg, from about 2.5 μg to about 200 μg, from about 3 μg to about 200 μg, from about 4 μg to about 200 μg, from about 5 μg to about 200 μg, from about 10 μg to about 200 μg, from about 15 μg to about 200 μg, from about 20 μg to about 200 μg, from about 25 μg to about 200 μg, from about 30 μg to about 200 μg, from about 35 μg to about 200 μg, from about 40 μg to about 200 μg, from about 45 μg to about 200 μg, from about 50 μg to about 200 μg, from about 55 μg to about 200 μg, from about 60 μg to about 200 μg, from about 65 μg to about 200 μg, from about 70 μg to about 200 μg, from about 75 μg to about 200 μg, from about 80 μg to about 200 μg, from about 85 μg to about 200 μg, from about 90 μg to about 200 μg, from about 95 μg to about 200 μg, from about 100 μg to about 200 μg, from about 110 μg to about 200 μg, from about 120 μg to about 200 μg, from about 130 μg to about 200 μg, from about 140 μg to about 200 μg, from about 150 μg to about 200 μg, from about 160 μg to about 200 μg, from about 170 μg to about 200 μg, from about 180 μg to about 200 μg, from about 190 μg to about 200 μg, from about 0.1 μg to about 190 μg, from about 0.1 μg to about 180 μg, from about 0.1 μg to about 170 μg, from about 0.1 μg to about 160 μg, from about 0.1 μg to about 150 μg, from about 0.1 μg to about 140 μg, from about 0.1 μg to about 130 μg, from about 0.1 μg to about 120 μg, from about 0.1 μg to about 110 μg, from about 0.1 μg to about 100 μg, from about 1 μg to about 150 μg, from about 5 μg to about 150 μg or from about 5 μg to about 120 μg. In certain embodiments, the drug delivery composition can include from about 1 μg to about 100 μg of the therapeutic agent, e.g., from about 1 μg to about 95 μg, from about 1 μg to about 90 μg, from about 1 μg to about 85 μg, from about 1 μg to about 80 μg, from about 1 μg to about 75 μg, from about 1 μg to about 70 μg, from about 1 μg to about 65 μg, from about 1 μg to about 60 μg, from about 1 μg to about 55 μg, from about 1 μg to about 50 μg, from about 1 μg to about 45 μg, from about 1 μg to about 40 μg, from about 1 μg to about 35 μg, from about 1 μg to about 30 μg, from about 1 μg to about 25 μg, from about 1 μg to about 20 μg, from about 1 μg to about 15 μg, from about 1 μg to about 14 μg, from about 1 μg to about 13 μg, from about 1 μg to about 12 μg, from about 1 μg to about 11 μg, from about 1 μg to about 10 μg, from about 1 μg to about 9 μg, from about 2 μg to about 100 μg, from about 3 μg to about 100 μg, from about 4 μg to about 100 μg, from about 5 μg to about 100 μg, from 5 about 6 μg to about 100 μg, from about 7 μg to about 100 μg, from about 8 μg to about 100 μg, from about 9 μg to about 100 μg, from about 10 μg to about 100 μg, from about 11 μg to about 100 μg, from about 12 μg to about 100 μg, from about 13 μg to about 100 μg, from about 14 μg to about 100 μg, from about 15 μg to about 100 μg, from about 16 μg to about 100 μg, from about 17 μg to about 100 μg, from about 18 μg to about 100 μg, from about 19 μg to about 100 μg, from about 20 μg to about 100 μg, from about 25 μg to about 100 μg, from about 30 μg to about 100 μg, from about 35 μg to about 100 μg, from about 40 μg to about 100 μg, from about 45 μg to about 100 μg, from about 50 μg to about 100 μg, from about 55 μg to about 100 μg, from about 60 μg to about 100 μg, from about 65 μg to about 100 μg, from about 70 μg to about 100 μg, from about 75 μg to about 100 μg, from about 80 μg to about 100 μg, from about 85 μg to about 100 μg, from about 90 μg to about 100 μg, from about 95 μg to about 100 μg, from about 5 μg to about 50 μg, from about 5 μg to about 45 μg, from about 5 μg to about 40 μg, from about 5 μg to about 35 μg, from about 5 μg to about 30 μg, from about 5 μg to about 25 μg, or from about 5 μg to about 20 μg. In certain embodiments, the drug delivery composition can include from about 1 μg to about 5 μg of the therapeutic agent. In certain embodiments, the drug delivery composition can include from about 1 μg to about 10 μg of the therapeutic agent. In certain embodiments, the drug delivery composition can include from about 1 μg to about 15 μg of the therapeutic agent. In certain embodiments, the drug delivery composition can include from about 1 μg to about 20 μg of the therapeutic agent. In certain embodiments, the drug delivery composition can include from about 5 μg to about 20 μg of the therapeutic agent. In certain embodiments, the drug delivery composition can include from about 1 μg to about 30 μg of the therapeutic agent. In certain embodiments, the drug delivery composition can include from about 5 μg to about 10 μg of the therapeutic agent. In certain embodiments, the drug delivery composition can include from about 5 μg to about 15 μg of the therapeutic agent. In certain embodiments, the drug delivery composition can include from about 5 μg to about 20 μg of the therapeutic agent. In certain embodiments, the drug delivery composition can include from about 5 μg to about 25 μg of the therapeutic agent. In certain embodiments, the drug delivery composition can include from about 5 μg to about 30 μg of the therapeutic agent. In certain embodiments, the drug delivery composition can include from about 0.1 μg to about 30 μg of the therapeutic agent. In certain embodiments, the drug delivery composition can include from about 0.1 μg to about 20 μg of the therapeutic agent. In certain embodiments, the drug delivery composition can include from about 0.1 μg to about 15 μg of the therapeutic agent. In certain embodiments, the drug delivery composition can include from about 0.1 μg to about 10 μg of the therapeutic agent. In certain embodiments, the drug delivery composition can include from about 0.1 μg to about 5 μg of the therapeutic agent.

In certain embodiments, the drug delivery composition can continuously release the therapeutic agent at a drug delivery rate (e.g., an average drug delivery rate) of about 0.01 μg/day to about 1 mg/day of the therapeutic agent, e.g., dexamethasone, or any values in between (e.g., when administered in close proximity to an analyte sensor and/or when incorporated into an analyte sensor). In certain embodiments, the drug delivery composition can continuously release the therapeutic agent at a drug delivery rate (e.g., an average drug delivery rate) of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, or about 1 mg/day of the therapeutic agent, e.g., dexamethasone, or any values in between (e.g., when administered in close proximity to an analyte sensor and/or when incorporated into an analyte sensor). In certain embodiments, the drug delivery composition can continuously release the therapeutic agent at a drug delivery rate (e.g., an average drug delivery rate) of about 0.2 μg/day to about 5 μg/day of the therapeutic agent, e.g. dexamethasone. In certain embodiments, the drug delivery composition can continuously release the therapeutic agent at a drug delivery rate (e.g., an average drug delivery rate) of about 0.2 μg/day to about 2 μg/day of the therapeutic agent, e.g., dexamethasone. In certain embodiments, the drug delivery composition can continuously release the therapeutic agent at a drug delivery rate (e.g., an average drug delivery rate) of about 0.2 μg/day to about 1 μg/day of the therapeutic agent, e.g., dexamethasone. In certain embodiments, the drug delivery composition can continuously release the therapeutic agent at a set or predetermined drug delivery rate to achieve desirable therapeutic results such as reducing, minimizing, reducing, preventing, and/or inhibiting the inflammation. In certain embodiments, the drug delivery composition can continuously release the therapeutic agent at a set or predetermined drug delivery rate to achieve desirable therapeutic results such as minimizing signal inaccuracy towards the end of sensor life. In certain embodiments, the drug delivery composition can continuously release the therapeutic agent at a set or predetermined drug delivery rate to reduce and/or minimize sensor signal inaccuracies or in vivo sensor failure, e.g., due to FBR. In certain embodiments, the drug delivery composition can continuously release the therapeutic agent at a set or predetermined drug delivery rate to achieve desirable therapeutic results such as minimizing and/or reducing LSA.

In certain embodiments, the drug delivery composition can continuously release the therapeutic agent at a set or predetermined drug delivery rate for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days (e.g., when administered in close proximity to an analyte sensor and/or when incorporated into an analyte sensor). In certain embodiments, the drug delivery composition can continuously release the therapeutic agent (e.g., at a set or predetermined drug delivery rate) for at least 5 days. In certain embodiments, the drug delivery composition can continuously release the therapeutic agent (e.g., at a set or predetermined drug delivery rate) for at least 10 days. In certain embodiments, the drug delivery composition can continuously release the therapeutic agent (e.g., at a set or predetermined drug delivery rate) for at least 15 days. In certain embodiments, the drug delivery composition can continuously release the therapeutic agent (e.g., at a set or predetermined drug delivery rate) for at least 20 days. In certain embodiments, the drug delivery composition can continuously release the therapeutic agent (e.g., at a set or predetermined drug delivery rate) for at least 21 days. In certain embodiments, the drug delivery composition can continuously release the therapeutic agent (e.g., at a set or predetermined drug delivery rate) for at least 25 days. In certain embodiments, the drug delivery composition may continuously release the therapeutic agent at a set or predetermined drug delivery rate for a set or predetermined days such as for at least 30 days.

In certain embodiments, a delivery composition of the present disclosure releases about 1% to about 100% of the therapeutic agent present within the composition (e.g., the total amount of therapeutic agent loaded into the composition), e.g., within a period of about 30-31 days (e.g., when administered in close proximity to an analyte sensor and/or when incorporated into an analyte sensor). In certain embodiments, a delivery composition of the present disclosure releases about 5% to about 100% of the therapeutic agent present within the composition (e.g., the total amount of therapeutic agent loaded into the composition), e.g., within a period of about 30-31 days. In certain embodiments, a delivery composition of the present disclosure releases about 10% to about 100% of the therapeutic agent present within the composition (e.g., the total amount of therapeutic agent loaded into the composition), e.g., within a period of about 30-31 days. In certain embodiments, a delivery composition of the present disclosure releases about 20% to about 100% of the therapeutic agent present within the composition (e.g., the total amount of therapeutic agent loaded into the composition), e.g., within a period of about 30-31 days. In certain embodiments, a delivery composition of the present disclosure releases about 30% to about 100% of the therapeutic agent present within the composition (e.g., the total amount of therapeutic agent loaded into the composition), e.g., within a period of about 30-31 days. In certain embodiments, a delivery composition of the present disclosure releases about 40% to about 100% of the therapeutic agent present within the composition (e.g., the total amount of therapeutic agent loaded into the composition), e.g., within a period of about 30-31 days. In certain embodiments, a delivery composition of the present disclosure releases about 50% to about 100% of the therapeutic agent present within the composition (e.g., the total amount of therapeutic agent loaded into the composition), e.g., within a period of about 30-31 days. In certain embodiments, a delivery composition of the present disclosure releases about 60% to about 100% of the therapeutic agent present within the composition (e.g., the total amount of therapeutic agent loaded into the composition), e.g., within a period of about 30-31 days. In certain embodiments, a delivery composition of the present disclosure releases about 70% to about 100% of the therapeutic agent present within the composition (e.g., the total amount of therapeutic agent loaded into the composition), e.g., within a period of about 30-31 days. In certain embodiments, a delivery composition of the present disclosure releases about 80% to about 100% of the therapeutic agent present within the composition (e.g., the total amount of therapeutic agent loaded into the composition), e.g., within a period of about 30-31 days. In certain embodiments, a delivery composition of the present disclosure releases about 30% to about 90% of the therapeutic agent present within the composition (e.g., the total amount of therapeutic agent loaded into the composition), e.g., within a period of about 30-31 days. In certain embodiments, a delivery composition of the present disclosure releases about 40% to about 90% of the therapeutic agent present within the composition (e.g., the total amount of therapeutic agent loaded into the composition), e.g., within a period of about 30-31 days. In certain embodiments, a delivery composition of the present disclosure releases about 50% to about 90% of the therapeutic agent present within the composition (e.g., the total amount of therapeutic agent loaded into the composition), e.g., within a period of about 30-31 days. In certain embodiments, a delivery composition of the present disclosure releases about 1% to about 80% of the therapeutic agent present within the composition (e.g., the total amount of therapeutic agent loaded into the composition), e.g., within a period of about 30-31 days. In certain embodiments, a delivery composition of the present disclosure releases about 10% to about 80% of the therapeutic agent present within the composition (e.g., the total amount of therapeutic agent loaded into the composition), e.g., within a period of about 30-31 days. In certain embodiments, a delivery composition of the present disclosure releases about 20% to about 80% of the therapeutic agent present within the composition (e.g., the total amount of therapeutic agent loaded into the composition), e.g., within a period of about 30-31 days. In certain embodiments, a delivery composition of the present disclosure releases about 30% to about 80% of the therapeutic agent present within the composition (e.g., the total amount of therapeutic agent loaded into the composition), e.g., within a period of about 30-31 days. In certain embodiments, a delivery composition of the present disclosure releases about 40% to about 80% of the therapeutic agent present within the composition (e.g., the total amount of therapeutic agent loaded into the composition), e.g., within a period of about 30-31 days. For example, but not by way by way of limitation, a delivery composition of the present disclosure releases about 45% to about 80% of the therapeutic agent, releases about 50% to about 80% of the therapeutic agent, releases about 55% to about 80% of the therapeutic agent, releases about 60% to about 80% of the therapeutic agent, releases about 65% to about 80% of the therapeutic agent, releases about 70% to about 80% of the therapeutic agent, releases about 75% to about 80% of the therapeutic agent, releases about 40% to about 75% of the therapeutic agent, releases about 40% to about 70% of the therapeutic agent, releases about 40% to about 65% of the therapeutic agent, releases about 40% to about 60% of the therapeutic agent, releases about 40% to about 55% of the therapeutic agent, releases about 40% to about 50% of the therapeutic agent, releases about 40% to about 45% of the therapeutic agent, releases about 45% to about 75% of the therapeutic agent, releases about 50% to about 75% of the therapeutic agent, releases about 55% to about 80% of the therapeutic agent or releases about 60% to about 75% of the therapeutic agent present within the composition (e.g., the total amount of therapeutic agent loaded into the composition), e.g., within a period of about 30-31 days. In certain embodiments, a period of about 30-31 days is the wear time of a sensor described herein. In certain embodiments, a period of about 30-31 days is the life span of a sensor described herein.

In certain embodiments, up to about 100% of the therapeutic agent present within the drug delivery composition (e.g., the total amount of therapeutic agent loaded into the composition) on an analyte sensor can be released no sooner than about 7 days before the sensor's end of life (e.g., end of wear time). In certain embodiments, up to about 90% of the therapeutic agent present within the drug delivery composition (e.g., the total amount of therapeutic agent loaded into the composition) on an analyte sensor can be released no sooner than about 7 days before the sensor's end of life (e.g., end of wear time). In certain embodiments, up to about 80% of the therapeutic agent present within the drug delivery composition (e.g., the total amount of therapeutic agent loaded into the composition) on an analyte sensor can be released no sooner than about 7 days before the sensor's end of life (e.g., end of wear time). In certain embodiments, up to about 70% of the therapeutic agent present within the drug delivery composition (e.g., the total amount of therapeutic agent loaded into the composition) on an analyte sensor can be released no sooner than about 7 days before the sensor's end of life (e.g., end of wear time). In certain embodiments, up to about 60% of the therapeutic agent present within the drug delivery composition (e.g., the total amount of therapeutic agent loaded into the composition) on an analyte sensor can be released no sooner than about 7 days before the sensor's end of life (e.g., end of wear time). In certain embodiments, up to about 50% of the therapeutic agent present within the drug delivery composition (e.g., the total amount of therapeutic agent loaded into the composition) on an analyte sensor can be released no sooner than about 7 days before the sensor's end of life (e.g., end of wear time). In certain embodiments, up to about 40% of the therapeutic agent present within the drug delivery composition (e.g., the total amount of therapeutic agent loaded into the composition) on an analyte sensor can be released no sooner than about 7 days before the sensor's end of life (e.g., end of wear time).

In certain embodiments, no more than about 90% of therapeutic agent present within the drug delivery composition (e.g., the total amount of therapeutic agent loaded into the composition) is released, e.g., within a period of about 30-31 days (e.g., when administered in close proximity to an analyte sensor and/or when incorporated into an analyte sensor). In certain embodiments, no more than about 95% of the therapeutic agent present within the composition (e.g., the total amount of therapeutic agent loaded into the composition) is released, e.g., within a period of about 30-31 days. In certain embodiments, no more than about 80% of the therapeutic agent present within the composition (e.g., the total amount of therapeutic agent loaded into the composition) is released, e.g., within a period of about 30-31 days. In certain embodiments, no more than about 75% of the therapeutic agent present within the composition (e.g., the total amount of therapeutic agent loaded into the composition) is released, e.g., within a period of about 30-31 days. In certain embodiments, no more than about 70% of the therapeutic agent present within the composition (e.g., the total amount of therapeutic agent loaded into the composition) is released, e.g., within a period of about 30-31 days. In certain embodiments, no more than about 65% of the therapeutic agent present within the composition (e.g., the total amount of therapeutic agent loaded into the composition) is released, e.g., within a period of about 30-31 days. In certain embodiments, no more than about 60% of the therapeutic agent present within the composition (e.g., the total amount of therapeutic agent loaded into the composition) is released, e.g., within a period of about 30-31 days. In certain embodiments, no more than about 55% of the therapeutic agent present within the composition (e.g., the total amount of therapeutic agent loaded into the composition) is released, e.g., within a period of about 30-31 days. In certain embodiments, no more than about 50% of the therapeutic agent present within the composition (e.g., the total amount of therapeutic agent loaded into the composition) is released, e.g., within a period of about 30-31 days.

In certain embodiments, no more than about 30%, 35%, 40%, 45%, 50%, 55%, 60%, 65%, 70% or 75% of the therapeutic agent present within the drug delivery composition (e.g., the total amount of therapeutic agent loaded into the composition) is released in the first 5, 6 or 7 days after insertion of the composition (e.g., when administered in close proximity to an analyte sensor and/or when incorporated into an analyte sensor). In certain embodiments, no more than about 30% of the therapeutic agent present within the composition (e.g., the total amount of therapeutic agent loaded into the composition) is released in the first 5 days after insertion of the composition. In certain embodiments, no more than about 30% of the therapeutic agent present within the composition (e.g., the total amount of therapeutic agent loaded into the composition) is released in the first 6 days after insertion of the composition. In certain embodiments, no more than about 30% of the therapeutic agent present within the composition (e.g., the total amount of therapeutic agent loaded into the composition) is released in the first 7 days after insertion of the composition. In certain embodiments, no more than about 35% of the therapeutic agent present within the composition (e.g., the total amount of therapeutic agent loaded into the composition) is released in the first 5 days after insertion of the composition. In certain embodiments, no more than about 35% of the therapeutic agent present within the composition (e.g., the total amount of therapeutic agent loaded into the composition) is released in the first 6 days after insertion of the composition. In certain embodiments, no more than about 35% of the therapeutic agent present within the composition (e.g., the total amount of therapeutic agent loaded into the composition) is released in the first 7 days after insertion of the composition. In certain embodiments, no more than about 40% of the therapeutic agent present within the composition (e.g., the total amount of therapeutic agent loaded into the composition) is released in the first 5 days after insertion of the composition. In certain embodiments, no more than about 40% of the therapeutic agent present within the composition (e.g., the total amount of therapeutic agent loaded into the composition) is released in the first 6 days after insertion of the composition. In certain embodiments, no more than about 40% of the therapeutic agent present within the composition (e.g., the total amount of therapeutic agent loaded into the composition) is released in the first 7 days after insertion of the composition. In certain embodiments, no more than about 45% of the therapeutic agent present within the composition (e.g., the total amount of therapeutic agent loaded into the composition) is released in the first 5 days after insertion of the composition. In certain embodiments, no more than about 45% of the therapeutic agent present within the composition (e.g., the total amount of therapeutic agent loaded into the composition) is released in the first 6 days after insertion of the composition. In certain embodiments, no more than about 45% of the therapeutic agent present within the composition (e.g., the total amount of therapeutic agent loaded into the composition) is released in the first 7 days after insertion of the composition. In certain embodiments, no more than about 50% of the therapeutic agent present within the composition (e.g., the total amount of therapeutic agent loaded into the composition) is released in the first 5 days after insertion of the composition. In certain embodiments, no more than about 50% of the therapeutic agent present within the composition (e.g., the total amount of therapeutic agent loaded into the composition) is released in the first 6 days after insertion of the composition. In certain embodiments, no more than about 50% of the therapeutic agent present within the composition (e.g., the total amount of therapeutic agent loaded into the composition) is released in the first 7 days after insertion of the composition. In certain embodiments, no more than about 55% of the therapeutic agent present within the composition (e.g., the total amount of therapeutic agent loaded into the composition) is released in the first 5 days after insertion of the composition. In certain embodiments, no more than about 55% of the therapeutic agent present within the composition (e.g., the total amount of therapeutic agent loaded into the composition) is released in the first 6 days after insertion of the composition. In certain embodiments, no more than about 55% of the therapeutic agent present within the composition (e.g., the total amount of therapeutic agent loaded into the composition) is released in the first 7 days after insertion of the composition. In certain embodiments, no more than about 60% of the therapeutic agent present within the composition (e.g., the total amount of therapeutic agent loaded into the composition) is released in the first 5 days after insertion of the composition. In certain embodiments, no more than about 60% of the therapeutic agent present within the composition (e.g., the total amount of therapeutic agent loaded into the composition) is released in the first 6 days after insertion of the composition. In certain embodiments, no more than about 60% of the therapeutic agent present within the composition (e.g., the total amount of therapeutic agent loaded into the composition) is released in the first 7 days after insertion of the composition. In certain embodiments, no more than about 65% of the therapeutic agent present within the composition (e.g., the total amount of therapeutic agent loaded into the composition) is released in the first 5 days after insertion of the composition. In certain embodiments, no more than about 65% of the therapeutic agent present within the composition (e.g., the total amount of therapeutic agent loaded into the composition) is released in the first 6 days after insertion of the composition. In certain embodiments, no more than about 65% of the therapeutic agent present within the composition (e.g., the total amount of therapeutic agent loaded into the composition) is released in the first 7 days after insertion of the composition. In certain embodiments, no more than about 70% of the therapeutic agent present within the composition (e.g., the total amount of therapeutic agent loaded into the composition) is released in the first 5 days after insertion of the composition. In certain embodiments, no more than about 70% of the therapeutic agent present within the composition (e.g., the total amount of therapeutic agent loaded into the composition) is released in the first 6 days after insertion of the composition. In certain embodiments, no more than about 70% of the therapeutic agent present within the composition (e.g., the total amount of therapeutic agent loaded into the composition) is released in the first 7 days after insertion of the composition. In certain embodiments, no more than about 75% of the therapeutic agent present within the composition (e.g., the total amount of therapeutic agent loaded into the composition) is released in the first 5 days after insertion of the composition. In certain embodiments, no more than about 75% of the therapeutic agent present within the composition (e.g., the total amount of therapeutic agent loaded into the composition) is released in the first 6 days after insertion of the composition. In certain embodiments, no more than about 75% of the therapeutic agent present within the composition (e.g., the total amount of therapeutic agent loaded into the composition) is released in the first 7 days after insertion of the composition.

In certain embodiments, no more than about 25% or 30% of the therapeutic agent present within the drug delivery composition (e.g., the total amount of therapeutic agent loaded into the composition) is released between about day 7 to about day 14 after insertion of the composition (e.g., when administered in close proximity to an analyte sensor and/or when incorporated into an analyte sensor).

In certain embodiments, no more than about 25% or 30% of the therapeutic agent present within the drug delivery composition (e.g., the total amount of therapeutic agent loaded into the composition) is released between about day 14 to about day 31 after insertion of the composition (e.g., when administered in close proximity to an analyte sensor and/or when incorporated into an analyte sensor).

In certain embodiments, when an exemplary drug delivery composition is in contact with a tissue or fluid (e.g., interstitial fluid) of a subject in need thereof, the drug delivery composition would adsorb water from the physiological around (e.g., surrounding) to form a hydrogel. In certain embodiments, a diffusion-controlled or selected drug release mechanism of a drug delivery composition can be used. For example, in certain embodiments, the copolymer can be a polyvinylpyridine-based copolymer. In certain embodiments, the therapeutic agent can be dexamethasone. In certain embodiments, because dexamethasone is a small molecule drug, the size of dexamethasone is much smaller than the mesh size of the hydrogel formed after the drug delivery composition in contact with the tissue of the patient, dexamethasone is released from the drug delivery composition through a diffusion process. For a polyvinylpyridine-only hydrogel, even with very dense crosslinking such as utilizing the crosslinker of the present disclosure, the mesh size will almost always be too big to limit dexamethasone diffusion, resulting in an uncontrolled drug releasing.

However, the addition of hydrophobic units/regions to the copolymer can slow the release of the therapeutic agent from the hydrogel. For example, but not by way of limitation, a polymer affinity-controlled drug release mechanism according to certain embodiments of the present disclosure can be used. In certain embodiments, the copolymer has a backbone including hydrophilic units and hydrophobic units, such as a polyvinylpyridine-co-polystyrene polymer. The hydrophobic therapeutic agent such as dexamethasone can interact with the hydrophobic units/regions of copolymer matrices through a non-polar intermolecular interaction, which slow the release of the therapeutic agent from the hydrogel. Therefore, the higher polymer affinity to the hydrophobic therapeutic agent, the slower the release rate of drug from the drug delivery composition, and the slower the drug delivery rate of the drug delivery composition. The term “polymer affinity” utilized herein refers to the strength of nonpolar intermolecular interaction between the hydrophobic units of the copolymer and the therapeutic agent. In certain embodiments, the hydrophobic unit can be aliphatic chains such as methyl, ethyl, propyl, etc., or aromatic rings such as phenyl.

In certain embodiments, the drug delivery rate of the drug delivery composition can be even more finely tuned by adjusting the polymer affinity and swellability of the copolymer. For example, in certain embodiments, water uptake can be controlled by amount of the crosslinker added in the drug delivery composition. In certain embodiments, the copolymer can be a polyvinylpyridine-based copolymer such as a polyvinylpyridine-co-polystyrene polymer. When the crosslinker, such as diglycidyl-PEG or glycerol triglycidyl ether, crosslinks the backbone of the copolymers through bonding to the nitrogen atom of pyridine units of copolymer, positive charges are formed on the backbone of the copolymer. The positive charges facilitate the water uptake when the drug delivery composition is in contact with the tissue to form a hydrogel. When the amount of the crosslinker in the drug delivery composition increases, the positive charges formed on the backbone of copolymer increase, which increases water osmosis, thus increasing the swellability of the drug delivery composition and diffusion of the therapeutic agent. As a result, the drug delivery rate of the drug delivery composition increases. As shown in Example 1, when the mer % of hydrophobic units of the copolymer increases, the polymer affinity to the hydrophobic therapeutic agent increases, which facilitates to slow the release of the therapeutic agent from the hydrogel, reducing the drug delivery rate of the drug delivery composition. Therefore, through balancing hydrophobic interactions with water uptake of the drug delivery composition, the drug delivery rate of the drug delivery composition can be finely tuned.

Additional information regarding polyvinylpyridine-based polymers is provided in U.S. Patent Publication No. 2003/0042137 (e.g., Formula 2b), the content (e.g., amount) of which is incorporated by reference herein in its entirety. Additional information regarding polyvinylpyridine-co-styrene copolymer is provided in U.S. Pat. No. 8,761,857, the content (e.g., amount) of which is incorporated by reference herein in its entirety. Additional information regarding polyvinylpyridine-based polymers and polyvinylpyridine-co-styrene copolymer are provided in U.S. Patent Publication No. 2022/0202322 (e.g., in schemes 3-1, 3-2 and 3-3), the content (e.g., amount, e.g., at paragraphs [0442] and [0457]) of which is incorporated by reference herein in its entirety.

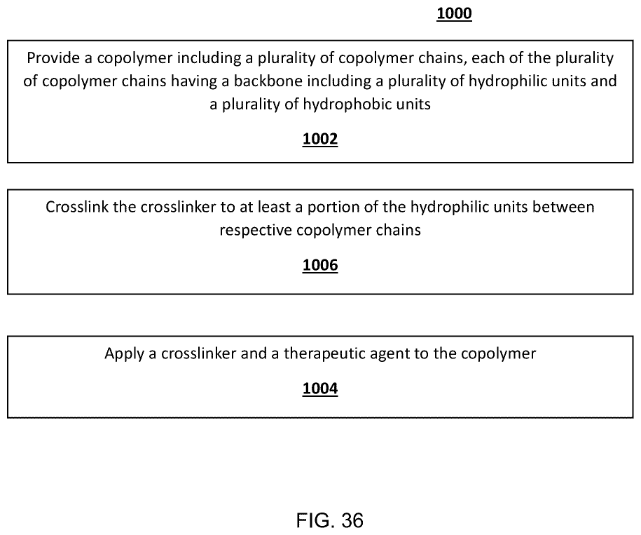

The present disclosure further provides a method of manufacturing a drug delivery composition described herein. Referring to FIG. 36, certain embodiments provide a method 1000 of manufacturing a drug delivery composition. In certain embodiments, the method 1000 can include: a task 1002 of providing a copolymer described herein (e.g., the copolymer includes a plurality of copolymer chains, wherein each of the plurality of copolymer chains comprises a backbone including a plurality of hydrophilic units and a plurality of hydrophobic units); a task 1004 of applying a crosslinker and a therapeutic agent to the copolymer; and a task 1006 of crosslinking the crosslinker to at least a portion of the hydrophilic units between respective copolymer chains.

### IV. Analyte Sensors

### A. General Structure of Analyte Sensor Systems

The present disclosure relates to the incorporation of therapeutic agents into an analyte sensor, e.g., an in vivo analyte sensor, and/or the delivery of a therapeutic composition in close proximity to an analyte sensor.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood analyte level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

FIG. 37 provides a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to certain embodiments. Reader device 120 can be a multi-purpose smartphone or a dedicated electronic reader instrument. While only one reader device 120 is shown, multiple reader devices 120 can be present in certain instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain embodiments, by individuals other than a primary user who have an interest in the user's analyte levels. Reader device 120 can include display 122 and optional input component 121. Display 122 can include a touch-screen interface, according to certain embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain embodiments.

### B. Analyte Sensor Tail

Sensor 104 of FIG. 37 is adapted to be at least partially inserted into a tissue of interest, such as within the dermal or subcutaneous layer of the skin. Sensor 104 can include a sensor tail of sufficient length for insertion to a desired depth in a given tissue. The sensor tail can include at least one working electrode. In certain embodiments, the sensor tail can include two working electrodes. In certain configurations, the sensor tail can include an active area for detecting an analyte, e.g., on a working electrode. A counter electrode can be present in combination with the at least one working electrode. Particular electrode configurations upon the sensor tail are described in more detail below.

The active area can be configured for detecting a particular analyte as described in further detail below. For example, but not by way of limitation, the analyte can be glucose, ketones, lactate, alcohol, glutamate, creatinine, sarcosine, ascorbate and a combination thereof. For example, but not by the way of limitation, a glucose-responsive active area can include a glucose-responsive enzyme, a ketones-responsive active area can include a ketones-responsive enzyme, a lactate-responsive active area can include a lactate-responsive enzyme, an alcohol-responsive active area can include an alcohol-responsive enzyme, a glutamate-responsive active area can include a glutamate-responsive enzyme, a creatine-responsive active area can include a creatine-responsive enzyme system, a sarcosine-responsive active area can include a sarcosine-responsive enzyme system, and an ascorbate-responsive active area can include an ascorbate-responsive enzyme system.

A membrane can overcoat the active area, as also described in further detail below. In certain embodiments, a membrane overcoating an analyte-responsive active area can function as a mass transport limiting membrane and/or to improve biocompatibility. A mass transport limiting membrane can act as a diffusion-limiting barrier to reduce the rate of mass transport of the analyte. For example, but not by way of limitation, limiting access of an analyte to the analyte-responsive active area with a mass transport limiting membrane can aid in avoiding sensor overload (saturation), thereby improving detection performance and accuracy. In certain embodiments, the membrane includes a copolymer of the present disclosure.

In certain embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain embodiments, analyte sensors of the present disclosure can be adapted for assaying dermal fluid or interstitial fluid to determine a concentration of one or more analytes in vivo. In certain embodiments, the biological fluid is interstitial fluid.

Referring still to FIG. 37, sensor 104 can automatically forward data to reader device 120. For example, but not by the way of limitation, analyte concentration data can be communicated automatically and periodically, such as at a certain frequency as data is obtained or after a certain time period has passed, with the data being stored in a memory until transmittal (e.g., every minute, five minutes, or other predetermined time period). In certain embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In certain embodiments, suitable needles can be solid or hollow, beveled or non-beveled, and/or circular or non-circular in cross-section. In more particular embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In certain embodiments, a tip of the needle (while present) can be angled over the terminus of sensor 104, such that the needle penetrates a tissue first and opens an access pathway for sensor 104. In certain embodiments, sensor 104 can reside within a lumen or groove of the needle, with the needle similarly opening an access pathway for sensor 104. In either case, the needle is subsequently withdrawn after facilitating sensor insertion.

i. Electrode Configuration

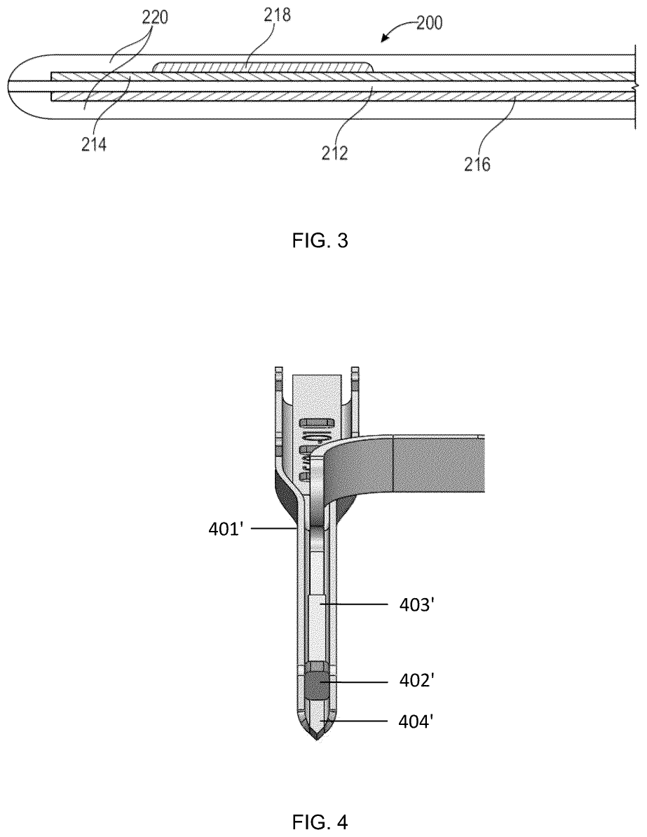

Sensor configurations featuring a single active area that is configured for detection of a corresponding single analyte can employ two-electrode or three-electrode detection motifs, as described further herein in reference to FIGS. 3 and 53A-53B. Sensor configurations featuring two different active areas for detection of the same or separate analytes, either upon separate working electrodes or upon the same working electrode, are described separately thereafter in reference to FIGS. 3 and 53A-55C. Sensor configurations having multiple working electrodes can be particularly advantageous for incorporating two different active areas within the same sensor tail, since the signal contribution from each active area can be determined more readily.

When a single working electrode is present in an analyte sensor, three-electrode sensor configurations can include a working electrode, a counter electrode and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape, substantially cylindrical in shape or any suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 3 shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Active area 218 is disposed as at least one layer upon at least a portion of working electrode 214. Active area 218 can include multiple spots or a single spot configured for detection of an analyte at a low working electrode potential, as discussed further herein. In certain embodiments, active area 218 can comprise an electron transfer agent described herein.

Referring still to FIG. 3, membrane 220 overcoats at least active area 218. In certain embodiments, membrane 220 comprises a copolymer of the present disclosure. For example, but not by way of limitation, membrane 220 comprises a copolymer comprising a first monomer, e.g., a styrene, and a second monomer comprising a heterocycle-containing component, e.g., a vinylpyridine, e.g., 4-vinylpyridine.

In certain embodiments, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to active area 218 (i.e., membrane 220 is a mass transport limiting membrane having some permeability for the analyte of interest). The composition and thickness of membrane 220 can vary to promote a desired analyte flux to active area 218, thereby providing a desired signal intensity and stability. Analyte sensor 200 can be operable for assaying an analyte by any of coulometric, amperometric, voltammetric, or potentiometric electrochemical detection techniques.

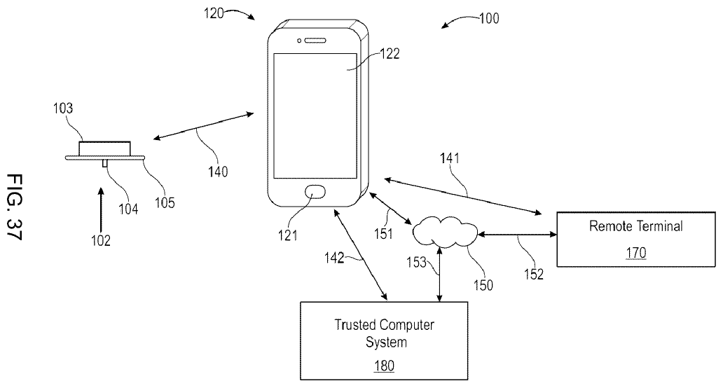

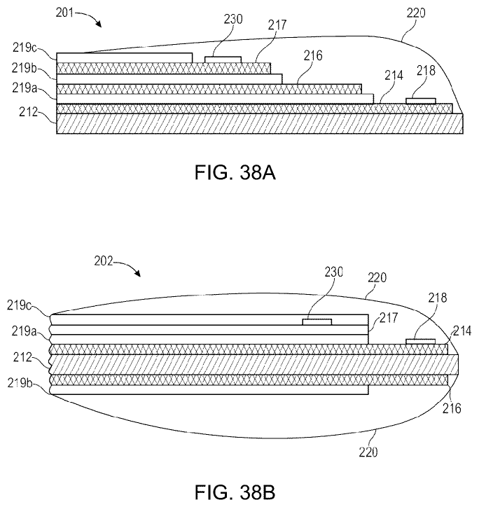

FIGS. 38A and 38B show diagrams of illustrative three-electrode analyte sensor configurations, which are also compatible for use in the disclosure herein. Three-electrode analyte sensor configurations can be similar to that shown for analyte sensor 200 in FIG. 3, except for the inclusion of additional electrode 217 in analyte sensors 201 and 202 (FIGS. 38A and 38B). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 38A, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 38B. Thus, in certain embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 38A and 38B. As with sensor 200 shown in FIG. 3, active area 218 in analyte sensors 201 and 202 can include multiple spots or a single spot. In certain embodiments, active area 218 can include a redox mediator disclosed herein. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat active area 218, as well as other sensor components, in analyte sensors 201 and 202, thereby serving as a mass transport limiting membrane. In certain embodiments, the additional electrode 217 can be overcoated with membrane 220. Although FIGS. 38A and 38B have depicted electrodes 214, 216 and 217 as being overcoated with membrane 220, it is to be recognized that in certain embodiments only working electrode 214 is overcoated. Moreover, the thickness of membrane 220 at each of electrodes 214, 216 and 217 can be the same or different. As in two-electrode analyte sensor configurations (FIG. 3), one or both faces of analyte sensors 201 and 202 can be overcoated with membrane 220 in the sensor configurations of FIGS. 38A and 38B, or the entirety of analyte sensors 201 and 202 can be overcoated. Accordingly, the three-electrode sensor configurations shown in FIGS. 38A and 38B should be understood as being non-limiting of the embodiments disclosed herein, with alternative electrode and/or layer configurations remaining within the scope of the present disclosure.

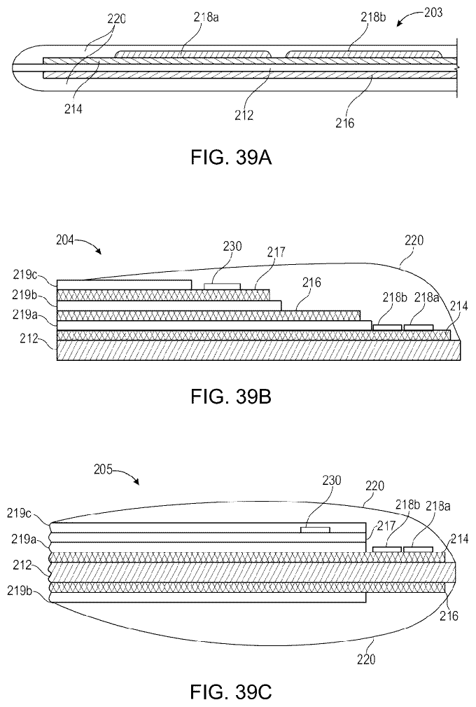

FIG. 39A shows an illustrative configuration for sensor 203 having a single working electrode with two different active areas disposed thereon. FIG. 39A is similar to FIG. 3, except for the presence of two active areas upon working electrode 214: first active area 218a and second active area 218b, which are responsive to the same or different analytes and are laterally spaced apart from one another upon the surface of working electrode 214. Active areas 218a and 218b can include multiple spots or a single spot configured for detection of each analyte. The composition of membrane 220 can vary or be compositionally the same at active areas 218a and 218b. First active area 218a and second active area 218b can be configured to detect their corresponding analytes at working electrode potentials that differ from one another, as discussed further below.

FIGS. 39B and 39C show cross-sectional diagrams of illustrative three-electrode sensor configurations for sensors 204 and 205, respectively, each featuring a single working electrode having first active area 218a and second active area 218b disposed thereon. FIGS. 39B and 39C are otherwise similar to FIGS. 39B and 39C and can be better understood by reference thereto. As with FIG. 39A, the composition of membrane 220 can vary or be compositionally the same at active areas 218a and 218b. In certain embodiments, any one of active areas 218a and 218b can comprise a redox mediator described herein. In certain embodiments, only one of active areas 218a and 218b can comprise a redox mediator described herein. For example, but not by way of limitation, only active area 218a includes a redox mediator described herein. In certain embodiments, only active area 218b includes a redox mediator described herein. In certain embodiments, both active areas 218a and 218b comprise a redox mediator described herein. In certain embodiments, the electron transfer agent present in active area 218a is different from the redox mediator present in 218b. Alternatively, the electron transfer agent present in active area 218a is the same redox mediator present in 218b.

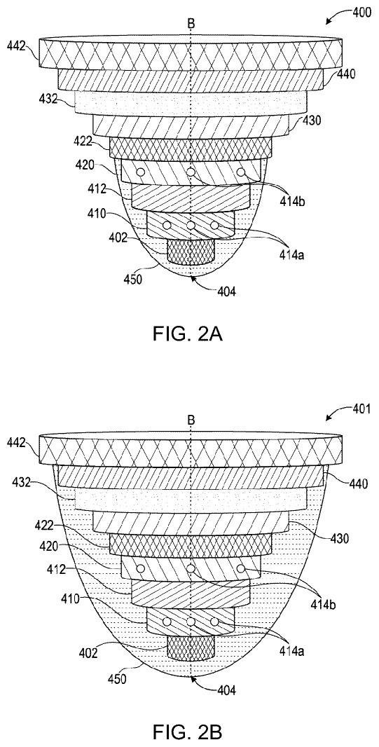

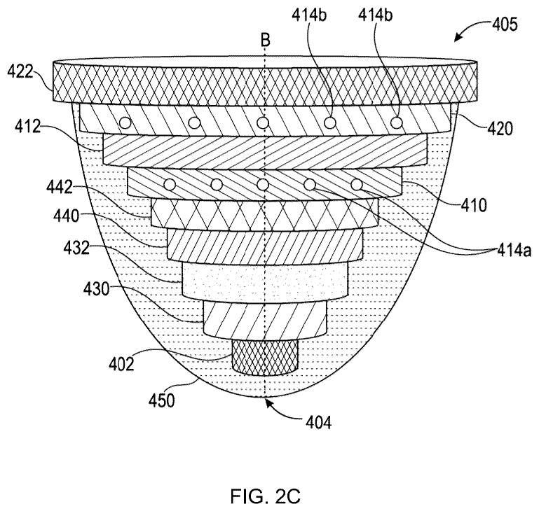

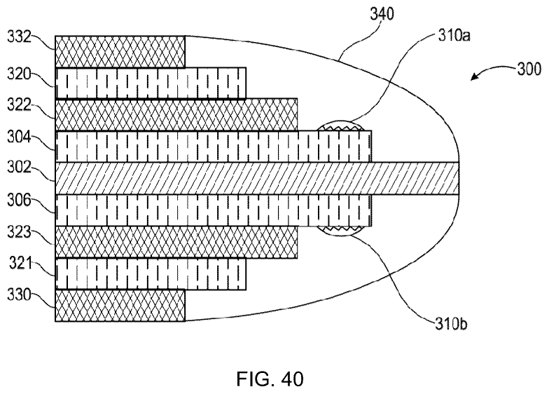

Illustrative sensor configurations having multiple working electrodes, specifically two working electrodes, are described in further detail in reference to FIGS. 2A-2C and FIG. 40. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte.

FIG. 40 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First active area 310a is disposed upon the surface of working electrode 304, and second active area 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least active areas 310a and 310b, according to various embodiments, with other components of analyte sensor 300 or the entirety of analyte sensor 300. In certain embodiments, membrane 340 comprises a copolymer of the present disclosure. For example, but not by way of limitation, membrane 340 comprises a copolymer comprising a first monomer, e.g., a styrene, and a second monomer comprising a heterocycle-containing component, e.g., a vinylpyridine, e.g., 4-vinylpyridine.

In certain embodiments, membrane 340 can be continuous but vary compositionally upon active area 310a and/or upon active area 310b in order to afford different permeability values for differentially regulating the analyte flux at each location. For example, but not by way of limitation, the one or more electrodes can be overcoated with a first membrane portion 340a and/or a second membrane portion 340b. In certain embodiments, different membrane formulations can be sprayed and/or printed onto the opposing faces of analyte sensor 300. Dip coating techniques can also be appropriate, particularly for depositing at least a portion of a bilayer membrane upon one of active areas 310a and 310b. In certain embodiments, membrane 340 can be the same or vary compositionally at active areas 310a and 310b. For example, but not by way of limitation, membrane 340 can include a bilayer overcoating active area 310a and be a homogeneous membrane overcoating active area 310b, or membrane 340 can include a bilayer overcoating active areas 310b and be a homogeneous membrane overcoating active area 310a. In certain embodiments, one of the first membrane portion and the second membrane portion can comprise a bilayer membrane and the other of the first membrane portion and the second membrane portion can comprise a single membrane polymer, according to particular embodiments of the present disclosure. In certain embodiments, an analyte sensor can include more than one membrane 340, e.g., two or more membranes. For example, but not by way of limitation, an analyte sensor can include a membrane that overcoats the one or more active areas, e.g., 310a and 310b, and an additional membrane that overcoats the entire sensor as shown in FIG. 40. In such configurations, a bilayer membrane can be formed over the one or more active areas, e.g., 310a and 310b. In certain embodiments, the two membranes can have different polymeric compositions. For example, but not by way of limitation, a first membrane can include a copolymer of the present disclosure and the second membrane can include a different polymer. In certain embodiments, any one of active areas 310a and 310b can comprise an electron transfer agent described herein. In certain embodiments, only one of active areas 310a and 310b can comprise a redox mediator described herein. For example, but not by way of limitation, only active area 310a includes a redox mediator described herein. In certain embodiments, only active area 310b includes a redox mediator described herein. In certain embodiments, both active areas 310a and 310b comprise a redox mediator described herein. In certain embodiments, the redox mediator present in active area 310a is different from the electron transfer agent present in 310b. Alternatively, the redox mediator present in active area 310a is the same electron transfer agent present in 310b.

Alternative sensor configurations having multiple working electrodes and differing from the configuration shown in FIG. 40 can feature a counter/reference electrode instead of separate counter and reference electrodes 320, 321, and/or feature layer and/or membrane arrangements varying from those expressly depicted. For example, and not by the way of limitation, the positioning of counter electrode 320 and reference electrode 321 can be reversed from that depicted in FIG. 40. In addition, working electrodes 304 and 306 need not necessarily reside upon opposing faces of substrate 302 in the manner shown in FIG. 40.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described hereinbelow. For example, but not by way of limitation, concentric working electrodes that are spaced apart along the length of a sensor tail can facilitate membrane deposition through sequential dip coating operations, in a similar manner to that described above for substantially planar sensor configurations. FIGS. 2A-2C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 2A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 2A, first active areas 414a and second active areas 414b, which are responsive to different analytes, are disposed upon the exposed surfaces of working electrodes 410 and 420, respectively, thereby allowing contact with a fluid to take place for sensing. Although active areas 414a and 414b have been depicted as three discrete spots in FIG. 2A, it is to be appreciated that fewer or greater than three spots, including a continuous layer of active area, can be present in alternative sensor configurations. In certain embodiments, any one of active areas 414a and 414b can comprise an electron transfer agent described herein. In certain embodiments, only one of active areas 414a and 414b can comprise a redox mediator described herein. For example, but not by way of limitation, only active area 414a includes a redox mediator described herein. In certain embodiments, only active area 414b includes a redox mediator described herein. In certain embodiments, both active areas 414a and 414b comprise a redox mediator described herein. In certain embodiments, the redox mediator present in active area 414a is different from the electron transfer agent present in 414b. Alternatively, the redox mediator present in active area 414a is the same electron transfer agent present in 414b.

In FIG. 2A, sensor 400 is partially coated with membrane 450 upon working electrodes 410 and 420 and active areas 414a and 414b disposed thereon. FIG. 2B shows an alternative sensor configuration in which the substantial entirety of sensor 401 is overcoated with membrane 450. Membrane 450 can be the same or vary compositionally at active areas 414a and 414b. For example, membrane 450 can include a bilayer overcoating active area 414a and be a homogeneous membrane overcoating active area 414b. In certain embodiments, membrane 450 comprises a copolymer of the present disclosure. For example, but not by way of limitation, membrane 450 comprises a copolymer comprising a first monomer, e.g., a styrene, and a second monomer comprising a heterocycle-containing component, e.g., a vinylpyridine, e.g., 4-vinylpyridine.

It is to be further appreciated that the positioning of the various electrodes in FIGS. 2A and 2B can differ from that expressly depicted. For example, the positions of counter electrode 430 and reference electrode 440 can be reversed from the depicted configurations in FIGS. 2A and 2B. Similarly, the positions of working electrodes 410 and 420 are not limited to those that are expressly depicted in FIGS. 2A and 2B. FIG. 2C shows an alternative sensor configuration to that shown in FIG. 2B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of active areas 414a and 414b (five discrete sensing spots illustratively shown in FIG. 2C), thereby facilitating an increased signal strength in some cases. Similarly, central substrate 402 can be omitted in any concentric sensor configuration disclosed herein, wherein the innermost electrode can instead support subsequently deposited layers.

In certain embodiments, one or more electrodes of an analyte sensor described herein is a wire electrode, e.g., a permeable wire electrode. In certain embodiments, the sensor tail comprises a working electrode and a reference electrode helically wound around the working electrode. In certain embodiments, an insulator is disposed between the working and reference electrodes. In certain embodiments, portions of the electrodes are exposed to allow reaction of the one or more enzymes with an analyte on the electrode. In certain embodiments, each electrode is formed from a fine wire with a diameter of from about 0.001 inches or less to about 0.010 inches or more. In certain embodiments, the working electrode has a diameter of from about 0.001 inches or less to about 0.010 inches or more, e.g., from about 0.002 inches to about 0.008 inches or from about 0.004 inches to about 0.005 inches. In certain embodiments, an electrode is formed from a plated insulator, a plated wire or bulk electrically conductive material. In certain embodiments, the working electrode comprises a wire formed from a conductive material, such as platinum, platinum-iridium, palladium, graphite, gold, carbon, conductive polymer, alloys or the like. In certain embodiments, the conductive material is a permeable conductive material. In certain embodiments, the electrodes can be formed by a variety of manufacturing techniques (e.g., bulk metal processing, deposition of metal onto a substrate or the like), the electrodes can be formed from plated wire (e.g., platinum on steel wire) or bulk metal (e.g., platinum wire). In certain embodiments, the electrode is formed from tantalum wire, e.g., covered with platinum.

In certain embodiments, the reference electrode, which can function as a reference electrode alone, or as a dual reference and counter electrode, is formed from silver, silver/silver chloride or the like. In certain embodiments, the reference electrode is juxtaposed and/or twisted with or around the working electrode. In certain embodiments, the reference electrode is helically wound around the working electrode. In certain embodiments, the assembly of wires can be coated or adhered together with an insulating material so as to provide an insulating attachment.

In certain embodiments, additional electrodes can be included in the sensor tail. For example, but not by way of limitation, an analyte sensor of the present disclosure can include a three-electrode system (a working electrode, a reference electrode and a counter electrode) and/or an additional working electrode (e.g., an electrode for detecting a second analyte). In certain embodiments where the sensor comprises two working electrodes, the two working electrodes can be juxtaposed around which the reference electrode is disposed upon (e.g., helically wound around the two or more working electrodes). In certain embodiments, the two or more working electrodes can extend parallel to each other. In certain embodiments, the reference electrode is coiled around the working electrode and extends towards the distal end (i.e., in vivo end) of the sensor tail. In certain embodiments, the reference electrode extends (e.g., helically) to the exposed region of the working electrode.

In certain embodiments, one or more working electrodes are helically wound around a reference electrode. In certain embodiments where two or more working electrodes are provided, the working electrodes can be formed in a double-, triple-, quad- or greater helix configuration along the length of the sensor tail (for example, surrounding a reference electrode, insulated rod or other support structure). In certain embodiments, the electrodes, e.g., two or more working electrodes, are coaxially formed. For example, but not by way limitation, the electrodes all share the same central axis.

In certain embodiments, the working electrode comprises a tube with a reference electrode disposed or coiled inside, including an insulator therebetween. Alternatively, the reference electrode comprises a tube with a working electrode disposed or coiled inside, including an insulator therebetween. In certain embodiments, a polymer (e.g., insulating) rod is provided, wherein the one or more electrodes (e.g., one or more electrode layers) are disposed upon (e.g., by electro-plating). In certain embodiments, a metallic (e.g., steel or tantalum) rod or wire is provided, coated with an insulating material (described herein), onto which the one or more working and reference electrodes are disposed upon. For example, but not by way of limitation, the present disclosure provides a sensor, e.g., a sensor tail, that comprises one or more tantalum wires, where a conductive material is disposed upon a portion of the one or more tantalum wires to function as a working electrode. In certain embodiments, the platinum-clad tantalum wire is covered with an insulating material, where the insulating material is partially covered with a silver/silver chloride composition to function as a reference and/or counter electrode.

In certain embodiments where an insulator is disposed upon the working electrode (e.g., upon the platinum surface of the electrode), a portion of the insulator can be stripped or otherwise removed to expose the electroactive surface of the working electrode. For example, but not by way of limitation, a portion of the insulator can be removed by hand, excimer lasing, chemical etching, laser ablation, grit-blasting or the like. Alternatively, a portion of the electrode can be masked prior to depositing the insulator to maintain an exposed electroactive surface area. In certain embodiments, the portion of the insulator that is stripped and/or removed can be from about 0.1 mm or less to about 2 mm or more in length, e.g., from about 0.5 mm to about 0.75 mm in length. In certain embodiments, the insulator is a non-conductive polymer. In certain embodiments, the insulator comprises parylene, fluorinated polymers, polyethylene terephthalate, polyvinylpyrrolidone, polyurethane, polyimide and other non-conducting polymers. In certain embodiments, glass or ceramic materials can also be used in the insulator layer. In certain embodiments, the insulator comprises parylene. In certain embodiments, the insulator comprises a polyurethane. In certain embodiments, the insulator comprises a polyurethane and polyvinylpyrrolidone.

ii. Sensing Chemistry

The analyte sensors of the present disclosure can include one or more enzymes for detecting one or more analytes. In certain embodiments, an active area of a presently disclosed analyte sensor, e.g., disposed upon a working electrode, can be configured for detecting one or more analytes. In certain embodiments, an active area comprises one or more enzymes for detecting an analyte. In certain embodiments, an analyte sensor of the present disclosure can include more than one active area, where each active area is configured to detect the same analyte or different analytes. In certain embodiments, the sensor does not include an enzyme and the analyte is directly oxidized at the working electrode.

In certain embodiments, an active area of a sensor of the present disclosure can comprise one or more enzymes to detect an analyte including, but not limited to, glucose, lactate, ketones (e.g., ketone bodies), glutamine, alcohols, aspartate, asparagine, glutamate, creatinine, hematocrit, acetoacetate, fructosamine, amylase, bilirubin, cholesterol, chorionic gonadotropin, creatine kinase (e.g., CK-MB), creatine, DNA, RNA, growth factors, growth hormones, hormones (e.g., thyroid stimulating hormone), steroids, vitamins (e.g., ascorbic acid), uric acid, neurochemicals (e.g., acetylcholine, norepinephrine and dopamine), oxygen, albumin, hemoglobin A1C, alkaline phosphatase, alanine transaminase, aspartate aminotransferase, blood urea nitrogen, sarcosine, prostate-specific antigen, prothrombin, thrombin, troponin, pyruvate, acetaldehyde, ascorbate, galactose, L-xylono-1,4-lactone, glutathione disulfide, hydrogen peroxide, linoleate, 1,3-bisphosphoglycerate, 6-phospho-D-glucono-1,5-lactone, hemoglobin, pharmaceutical drugs (e.g., antibiotics (e.g., gentamicin, vancomycin and the like), digitoxin, digoxin, theophylline, insulin and warfarin), drugs of abuse (e.g., analgesics, depressants, stimulants and hallucinogens), metal ions (e.g., potassium, sodium, calcium, magnesium, manganese, iron, cobalt, molybdenum, zinc and chlorine), pH, carbonate, phosphate, sulfate, fatty acids and antibodies. In certain embodiments, the analyte is glucose, glutamate, creatinine, sarcosine and/or ascorbate. In certain embodiments, the analyte is glucose. In certain embodiments, the analyte is glutamate. In certain embodiments, the one or more enzymes in an active area of a sensor of the present disclosure can be used in detecting glutamate, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, and uric acid.

In certain embodiments, the one or more enzymes can include multiple enzymes, e.g., an enzyme system, that are collectively responsive to the analyte.

In certain embodiments, the enzyme can be an oxidoreductase. In certain embodiments, the oxidoreductase can be an enzyme belonging to enzyme class 1. For example, but not by way of limitation, the enzyme can belong to enzyme class 1.1 (e.g., 1.1.1 or 1.1.3), enzyme class 1.4 (e.g., 1.4.3) or enzyme class 1.5. In certain embodiments, the enzyme can be a NAD(P)+-dependent dehydrogenase. In certain embodiments, the enzyme can be a flavin adenine dinucleotide (FAD)-dependent oxidoreductase. In certain embodiments, the enzyme can be a hydrolase. In certain embodiments, the hydrolase can be an enzyme belonging to enzyme class 3. For example, but not by way of limitation, the enzyme can belong to enzyme class 3.5, e.g., 3.5.2 or 3.5.3.

In certain embodiments, an active area of a sensor of the present disclosure can include one or more enzymes that can be utilized to detect glucose. For example, but not by way of limitation, an analyte sensor of the present disclosure can include one or more enzymes for detecting glucose. In certain embodiments, the analyte sensor can include a glucose oxidase and/or a glucose dehydrogenase for detecting glucose. In certain embodiments, the analyte sensor can include a glucose oxidase. In certain embodiments, the glucose dehydrogenase can be a pyrroloquinoline quinone (PQQ) or a cofactor-dependent glucose dehydrogenase, e.g., flavin adenine dinucleotide (FAD)-dependent glucose dehydrogenase or nicotinamide adenine dinucleotide (NAD)-dependent glucose dehydrogenase. In certain embodiments, the active area can further include diaphorase. In certain embodiments, the enzyme for detecting glucose is an FAD-dependent glucose oxidase.

In certain embodiments, an active area of a sensor of the present disclosure can include one or more enzymes that can be utilized to detect ketones. For example, but not by way of limitation, an analyte sensor of the present disclosure can include one or more enzymes, e.g., an enzyme system, for detecting ketones. In certain embodiments, a ketones-responsive active area can include an enzyme system comprising multiple enzymes that are capable of acting in concert to facilitate detection of ketones, as described in U.S. Patent Publication No. 2020/0237275 (the contents of which are incorporated by reference herein in their entirety). In certain embodiments, the analyte sensor can include β-hydroxybutyrate dehydrogenase. In certain embodiments, the active area can further include diaphorase. In certain embodiments, the analyte sensor can include β-hydroxybutyrate dehydrogenase and diaphorase for detecting ketones.

In certain embodiments, an active area of a sensor of the present disclosure can include one or more enzymes that can be utilized to detect lactate. For example, but not by way of limitation, an analyte sensor of the present disclosure can include one or more enzymes, e.g., an enzyme system, for detecting lactate. In certain embodiments, a lactate-responsive active area can include an enzyme system comprising multiple enzymes that are capable of acting in concert to facilitate detection of lactate, as described in U.S. Publication No. 2019/0320947 (the contents of which are incorporated by reference herein in their entirety). In certain embodiments, the analyte sensor can include a lactate dehydrogenase. In certain embodiments, the analyte sensor can include a lactate oxidase. In certain embodiments, the active area can further include diaphorase. In certain embodiments, the analyte sensor can include a lactate oxidase and diaphorase.

In certain embodiments, an active area of a sensor of the present disclosure can include one or more enzymes that can be utilized to detect alcohol. For example, but not by way of limitation, an analyte sensor of the present disclosure can include one or more enzymes, e.g., an enzyme system, for detecting alcohol. In certain embodiments, an ethanol-responsive active area can include an enzyme system comprising multiple enzymes that are capable of acting in concert to facilitate detection of ethanol, as in U.S. Patent Publication No. 2020/0237277 (the contents of which are incorporated by reference herein in their entirety). In certain embodiments, the analyte sensor can include an alcohol dehydrogenase or a ketoreductase.

In certain embodiments, an active area of a sensor of the present disclosure can include one or more enzymes that can be utilized to detect creatinine. For example, but not by way of limitation, an analyte sensor of the present disclosure can include one or more enzymes, e.g., an enzyme system, for detecting creatinine. In certain embodiments, a creatinine-responsive active area can include an enzyme system comprising multiple enzymes that are capable of acting in concert to facilitate detection of creatinine, e.g., as described in U.S. Patent Publication No. 2020/0241015 (the contents of which are incorporated by reference herein in their entirety). In certain embodiments, the analyte sensor can include an amidohydrolase, creatine amidinohydrolase and/or sarcosine oxidase.

In certain embodiments, an active area of a sensor of the present disclosure can include one or more enzymes that can be utilized to detect glutamate. For example, but not by way of limitation, an analyte sensor of the present disclosure can include one or more enzymes, e.g., an enzyme system, for detecting glutamate. In certain embodiments, the analyte sensor can include a glutamate dehydrogenase or a glutamate oxidase.

In certain embodiments, an active area of the present disclosure can include one or more sensing spots (e.g., as shown in 218a and 218b in FIG. 39A), where each sensing spot can include the one or more enzymes for detecting an analyte.

In certain embodiments, an active area can further include a stabilizing agent, e.g., for stabilizing the one or more enzymes. For example, but not by way of limitation, the stabilizing agent can be an albumin, e.g., a serum albumin. Non-limiting examples of serum albumins can include bovine serum albumin and human serum albumin. In certain embodiments, the stabilizing agent can be a human serum albumin. In certain embodiments, the stabilizing agent can a bovine serum albumin.

In certain embodiments, an active area can further include a cofactor or coenzyme for one or more enzymes present in the active area. In certain embodiments, the cofactor can be nicotinamide adenine dinucleotide (NAD) or nicotinamide adenine dinucleotide phosphate (NADP). In certain embodiments, the coenzyme can be NAD.

In certain embodiments, a sensor of the present disclosure does not include an analyte-responsive active area comprising an enzyme. In certain embodiments, a sensor of the present disclosure includes a working electrode that does not have an enzyme disposed upon the working electrode or includes an inactive enzyme, e.g., an enzyme that lacks enzymatic activity (e.g., for the analyte of interest), disposed upon the working electrode. In certain embodiments, such a sensor can be used to detect an analyte that can be directly oxidized at the working electrode. For example, but not by way of limitation, a sensor of the present disclosure for detecting ascorbate does not include an enzyme on the working electrode. In certain embodiments, ascorbate is directly oxidized at the working electrode resulting in a signal that correlates to the level of ascorbate in the biological fluid contacting the sensor.

In certain embodiments, a working electrode that does not include an enzyme or includes an inactive enzyme can be used for detecting a background signal. In certain embodiments, the background signal includes a signal that is caused by chemical species other than the analyte of interest present in the sample, e.g., signal caused by an interferent. In certain embodiments, the background signal is a signal caused by one or more interferents. Non-limiting examples of interferents include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea and uric acid. In certain embodiments, the background signal can be used to calibrate, filter and/or normalize the signal obtained from a second working electrode (which is configured for detecting an analyte) present on the same analyte sensor. In certain embodiments, the signal from the working electrode that does not have enzyme (or has inactive enzyme) can be subtracted from the signal obtained from a working electrode that is configured to detect an analyte to determine the signal contribution from the analyte.

In certain embodiments, an analyte sensor disclosed herein can include an electron transfer agent. For example, but not by way of limitation, an active area can include an electron transfer agent. In certain embodiments, the presence of an electron transfer agent in an active area can depend on the enzyme or enzyme system used to detect the analyte and/or the composition of the working electrode.

Suitable electron transfer agents for use in the presently disclosed analyte sensors can facilitate conveyance of electrons to the adjacent working electrode after an analyte undergoes an enzymatic oxidation-reduction reaction within the active area, thereby generating a current that is indicative of the presence of that particular analyte. The amount of current generated is proportional to the quantity of analyte that is present.

In certain embodiments, suitable electron transfer agents can include electroreducible and electrooxidizable ions, complexes, or molecules (e.g., quinones) having oxidation-reduction potentials that are a few hundred millivolts above or below the oxidation-reduction potential of the standard calomel electrode. In certain embodiments, the redox mediators can include osmium complexes and other transition metal complexes, such as those described in U.S. Pat. Nos. 6,134,461 and 6,605,200, which are incorporated herein by reference in their entireties. Additional examples of suitable redox mediators can include those described in U.S. Pat. Nos. 6,736,957, 7,501,053 and 7,754,093, the disclosures of each of which are also incorporated herein by reference in their entireties. Other examples of suitable redox mediators can include metal compounds or complexes of ruthenium, osmium, iron (e.g. polyvinylferrocene or hexacyanoferrate), or cobalt, including metallocene compounds thereof, for example. Suitable ligands for the metal complexes can include, for example, bidentate or higher denticity ligands such as, for example, bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate or higher denticity ligands can be present in a metal complex, e.g., osmium complex, to achieve a full coordination sphere. In certain embodiments, the electron transfer agent is an osmium complex. In certain embodiments, the electron transfer agent is osmium complexed with bidentate ligands. In certain embodiments, the electron transfer agent is osmium complexed with tridentate ligands.

In certain embodiments, electron transfer agents disclosed herein can comprise suitable functionality to promote covalent bonding to a polymer (also referred to herein as a polymeric backbone) within the active areas as discussed further below. For example, but not by way of limitation, an electron transfer agent for use in the present disclosure can include a polymer-bound electron transfer agent, e.g., a redox polymer. Suitable non-limiting examples of polymer-bound electron transfer agents include those described in U.S. Pat. Nos. 8,444,834, 8,268,143 and 6,605,201 and U.S. Patent Publication No. 2022/0202326, the disclosures of which are incorporated herein by reference in their entirety. In certain embodiments, the electron transfer agent is a bidentate osmium complex bound to a polymer described herein, e.g., a polymeric backbone described in Section 4 below. In certain embodiments, the electron transfer agent is a tridentate osmium complex bound to a polymer described herein, e.g., a polymeric backbone described in Section 4 below. In certain embodiments, the polymer-bound electron transfer agent shown in FIG. 3 of U.S. Pat. No. 8,444,834 (referred to as “X7”) can be used in a sensor of the present disclosure.

In certain embodiments, one or more working electrodes of an analyte sensor of the present disclosure does not have a redox mediator disposed upon the working electrode. In certain embodiments, one or more working electrodes of an analyte sensor of the present disclosure does not have a redox mediator or an enzyme disposed upon the working electrode. In certain embodiments, such working electrodes can be used to detect an analyte that can be directly oxidized at the working electrode.

iii. Mass-Limiting Membrane

In certain embodiments, an analyte sensor of the present disclosure further includes a membrane covering at least a portion of the sensing layer. For example, but not by way of limitation, the membrane can function as a mass-limiting membrane and/or to improve biocompatibility. In certain embodiments, membrane (e.g., 220 in FIG. 3) can overcoat at least a portion of the active area.

A mass-limiting membrane can act as a diffusion-limiting barrier to reduce the rate of mass transport of the analyte, e.g., glucose, an alcohol, a ketone, or lactate, when the sensor is in use. For example, but not by way of limitation, limiting access of an analyte, e.g., glucose, to the sensing spot with a mass-limiting membrane can aid in avoiding sensor overload (saturation), thereby improving detection performance and accuracy. In certain embodiments, the mass-limiting layer can limit the flux of an analyte to the working electrode in an electrochemical sensor so that the sensor is linearly responsive over a large range of analyte concentrations.

In certain embodiments, the mass-limiting membrane can be homogeneous and can be single-component (contain a single membrane polymer). In certain embodiments, the mass-limiting membrane can be multi-component (contain two or more different membrane polymers). In certain embodiments, the multi-component membrane can be present as a bilayer membrane or as a homogeneous admixture of two or more membrane polymers. A homogeneous admixture can be deposited by combining the two or more membrane polymers in a solution and then depositing the solution upon a working electrode, e.g., by dip coating.

In certain embodiments, the mass-limiting membrane can include two or more layers, e.g., a bilayer or tri-layer membrane. In certain embodiments, each layer can include a different polymer or the same polymer at different concentrations or thicknesses.

In certain embodiments, a mass-limiting membrane can include polymers containing heterocyclic nitrogen groups. In certain embodiments, a mass-limiting membrane can include a polyvinylpyridine-based polymer. Non-limiting examples of polyvinylpyridine-based polymers are disclosed in U.S. Patent Publication No. 2003/0042137, the content of which is incorporated by reference herein in its entirety. In certain embodiments, the polyvinylpyridine-based polymer has a molecular weight from about 50 kD to about 500 kD, e.g., from about 50 kD to about 200 kD.

In certain embodiments, a mass-limiting membrane can include a polyvinylpyridine (e.g., poly(2-vinylpyridine) or poly(4-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and the like.

In certain embodiments, a mass-limiting membrane can include a polyvinylpyridine (e.g., poly(4-vinylpyridine) and/or poly(2-vinylpyridine)). In certain embodiments, a mass-limiting membrane can include poly(4-vinylpyridine). In certain embodiments, a mass-limiting membrane can include a copolymer of vinylpyridine and styrene. In certain embodiments, the mass-limiting membrane can include a polyvinylpyridine-co-styrene copolymer. For example, but not by way of limitation, a polyvinylpyridine-co-styrene copolymer can include a polyvinylpyridine-co-styrene copolymer in which a portion of the pyridine nitrogen atoms are functionalized with a non-crosslinked polyethylene glycol tail and a portion of the pyridine nitrogen atoms were functionalized with an alkylsulfonic acid group, e.g., a propylsulfonic acid. In certain embodiments, a derivatized polyvinylpyridine-co-styrene copolymer for use as a membrane polymer can be the 10Q5 polymer as described in U.S. Pat. No. 8,761,857, the content of which is incorporated by reference herein in its entirety.

A suitable copolymer of vinylpyridine and styrene can have a styrene content ranging from about 0.01% to about 50% mole percent (mer %), or from about 0.05% to about 45% mole percent, or from about 0.1% to about 40% mole percent, or from about 0.5% to about 35% mole percent, or from about 1% to about 30% mole percent, or from about 2% to about 25% mole percent, or from about 5% to about 20% mole percent. In certain embodiments, a copolymer of vinylpyridine and styrene can include a styrene content ranging from about 2% to about 25% mole percent. Substituted styrene can be utilized similarly and in similar amounts.

A suitable copolymer of vinylpyridine and styrene can have a weight average molecular weight of 5 kD or more, or about 10 kD or more, or about 15 kD or more, or about 20 kD or more, or about 25 kD or more, or about 30 kD or more, or about 40 kD or more, or about 50 kD or more, or about 75 kD or more, or about 90 kD or more, about 100 kD or more or about 110 kD or more. In non-limiting examples, a suitable copolymer of vinylpyridine and styrene can have a weight average molecular weight ranging from about 5 kD to about 150 kD, or from about 10 kD to about 125 kD, or from about 15 kD to about 100 kD, or from about 20 kD to about 80 kD, or from about 25 kD to about 75 kD, or from about 30 kD to about 60 kD. In certain embodiments, a copolymer of vinylpyridine and styrene can have a weight average molecular weight ranging from about 10 kD to about 125 kD.

In certain embodiments, a mass-limiting membrane can further include a silicone polymer, e.g., a polydimethylsiloxane (PDMS). For example, but not by way of limitation, a mass-limiting membrane can include a polyvinylpyridine-co-styrene copolymer (e.g., a derivatized polyvinylpyridine-co-styrene copolymer) and a silicone polymer (e.g., a poly dimethylsiloxane (PDMS)).

iv. Interference Domain

In certain embodiments, the analyte sensor of the present disclosure can further include an interference domain. For example, but not by way of limitation, a sensor tail 100 or 200 of an analyte sensor can further comprise an interference domain. In certain embodiments, the interference domain can include a polymer domain that restricts the flow of one or more interferants, e.g., to the surface of the working electrode. In certain embodiments, the interference domain can function as a molecular sieve that allows analytes and other substances that are to be measured by the working electrode to pass through, while preventing passage of other substances such as interferents. In certain embodiments, the interferents can affect the signal obtained at the working electrode. Non-limiting examples of interferents can include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea, and uric acid.

In certain embodiments, the interference domain is located between the working electrode and the active area. In certain embodiments, non-limiting examples of polymers that can be utilized in the interference domain can include polyurethanes, polymers having pendant ionic groups, and polymers having controlled pore size. In certain embodiments, the interference domain can be formed from one or more cellulosic derivatives.

Non-limiting examples of cellulosic derivatives include polymers such as cellulose acetate, cellulose acetate butyrate, 2-hydroxyethyl cellulose, cellulose acetate phthalate, cellulose acetate propionate, cellulose acetate trimellitate and the like.

In certain embodiments, the interference domain is part of the mass-limiting membrane and not a separate membrane. In certain embodiments, the interference domain is located between the one or more sensing spots and the mass-limiting membrane.

In certain embodiments, the interference domain can include a thin, hydrophobic membrane that is non-swellable and restricts diffusion of high molecular weight species. For example, but not by way of limitation, the interference domain can be permeable to relatively low molecular weight substances, such as hydrogen peroxide, while restricting the passage of higher molecular weight substances, such as ketones, glucose, acetaminophen and/or ascorbic acid.

### C. Incorporation of Drug Delivery Compositions

The present disclosure further provides an analyte sensor that includes a drug delivery composition described herein (e.g., one or more drug delivery compositions disclosed herein). The present disclosure provides an analyte sensor of the present disclosure comprising a sensor tail (e.g., 200 in FIG. 3) that further comprises a drug delivery composition. Non-limiting examples of drug delivery compositions that can be included in an analyte sensor disclosed herein is described in Section III and therapeutic agents that can be included in a drug delivery composition is described in Section II.

The incorporation of a therapeutic agent within the analyte sensor itself allows for targeted delivery of the therapeutic agent to the tissue surrounding the implantation site and the analyte sensor and allows for the release of the therapeutic agent in close proximity to the analyte sensor in vivo. In certain embodiments, the therapeutic agent to be delivered according to the present disclosure can be a therapeutic agent that is effective at reducing, minimizing, preventing and/or inhibiting a tissue's response to analyte sensor implantation and/or a tissue infection, thus, to prevent and/or reduce analyte signal inaccuracy towards the end of sensor life. In certain embodiments, the therapeutic agent to be delivered according to the present disclosure can be a therapeutic agent that is effective at reducing, minimizing, preventing and/or inhibiting a tissue's response to analyte sensor implantation and/or a tissue infection, thus, to prevent and/or reduce LSA.

The present disclosure provides an analyte sensor of the present disclosure, e.g., a sensor tail, further comprising a drug delivery composition. FIGS. 2-3 and 38-40C illustrate cross-sectional diagrams of an exemplary analyte sensor according to certain embodiments of the present disclosure. As shown in FIG. 3, the analyte sensor can include: (i) a sensor tail 200 including at least a first working electrode 214 on a substrate 212; (ii) an active area 218 disposed upon a surface of the first working electrode for detecting an analyte; (iii) a mass transport limiting membrane 220 permeable to the analyte that overcoats at least the active area; (iv) a counter/reference electrode 216 on the substrate 212; and (v) a drug delivery composition including (a) a copolymer including a plurality of copolymer chains, wherein each of the plurality of copolymer chains comprises a backbone including a plurality of hydrophilic units and a plurality of hydrophobic units, (b) a crosslinker crosslinking at least a portion of the hydrophilic units between respective copolymer chains, and (c) a therapeutic agent (e.g., where the therapeutic agent is not covalently bonded to the copolymer).

In certain embodiments, the analyte sensor that includes a drug delivery composition is configured to detect glucose. In certain embodiments, the analyte sensor that includes a drug delivery composition is configured to detect glucose and ketones, e.g., on a first working electrode and a second working electrode, respectively. In certain embodiments, the analyte sensor that includes a drug delivery composition is configured to detect lactate. In certain embodiments, the analyte sensor that includes a drug delivery composition is configured to detect creatinine. In certain embodiments, the analyte sensor that includes a drug delivery composition is configured to detect ketones. In certain embodiments, the analyte sensor that includes a drug delivery composition is configured to detect alcohol.

In certain embodiments, the analyte sensor that includes a drug delivery composition is a dermal sensor.

In certain embodiments, the analyte sensor that includes a drug delivery composition is a subcutaneous sensor such as a subcutaneously implanted sensor. In certain embodiments, the analyte sensor that includes a drug delivery composition is an analyte sensor that detects an analyte in the interstitial fluid of a subject.

In certain embodiments, the analyte sensor that includes a drug delivery composition is an intravenous sensor such as intravenously implanted sensor.

In certain embodiments, the drug delivery composition can be disposed upon a structure or component of an analyte sensor. In certain embodiments, a drug delivery composition can be incorporated into an analyte sensor of the present disclosure. For example, but not by way of limitation, a drug delivery composition of the present disclosure can be disposed upon or incorporated into a component of the analyte sensor, e.g., a component of the sensor tail of an analyte sensor. In certain embodiments, the drug delivery composition can be disposed upon a structure or component of an analyte sensor. For example, but not by way of limitation, the drug delivery composition can be disposed upon a surface of an electrode (e.g., a counter/reference electrode (e.g., 216 of FIG. 38A) and/or a working electrode (e.g., 214 of FIG. 38A)), an insulating material (e.g., a dielectric material (e.g., 219a-c of FIG. 38A)), a substrate (e.g., 212 of FIG. 38A) and/or a mass transport limiting membrane (e.g., 220 of FIG. 38A).

In certain embodiments, the drug delivery composition can be disposed on the working electrode. In certain embodiments, the drug delivery composition can be disposed on the counter/reference electrode. In certain embodiments where the analyte sensor includes a counter electrode and a reference electrode, the composition (e.g., drug delivery composition) can be disposed on the counter/reference electrode. In certain embodiments, the drug composition (e.g., drug delivery composition) can be disposed on the counter electrode. In certain embodiments, the composition (e.g., drug delivery composition) can be disposed on the reference electrode. In certain embodiments where the analyte sensor includes a counter electrode and a reference electrode, the composition (e.g., drug delivery composition) can be disposed on the counter electrode. In certain embodiments where the analyte sensor includes a counter electrode and a reference electrode, the composition (e.g., drug delivery composition) can be disposed on the reference electrode.

In certain embodiments, the drug delivery composition can be disposed on the mass transport limiting membrane 220.

In certain embodiments, the hydrophilic unit of the copolymer of the drug delivery composition disposed upon the analyte sensor can include a nitrogen-containing heterocyclic unit such as a pyridine unit, a pyridazine unit, a pyrimidine unit, a pyrazine unit, a triazine unit, an imidazole unit, a pyrazole unit, etc. In certain embodiments, the hydrophobic unit of the copolymer of the drug delivery composition disposed upon the analyte sensor can include a non-heteroatom containing aromatic unit such as a benzene (phenyl) unit, a naphthalene unit, an anthracene unit, etc., an acyclic aliphatic unit such as a straight or branched alkyl unit, a straight or branched alkenyl unit, a straight or branched alkynyl unit, etc., and/or a cyclic aliphatic unit such as a cyclobutyl, a cyclopentyl unit, a cyclohexyl unit, a cycloheptyl unit, a cyclooctyl unit, a cyclohexenyl unit, etc.

In certain embodiments, the copolymer of the drug delivery composition disposed upon the analyte sensor can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.

In certain embodiments, the copolymer of the drug delivery composition disposed upon the analyte sensor can include a block polymer.

In certain embodiments, the copolymer of the drug delivery composition disposed upon the analyte sensor is a polyvinylimidazole-based copolymer. In certain embodiments, the polyvinylimidazole-based copolymer can be a copolymer of vinylimidazole and styrene or a derivative thereof.

In certain embodiments, the polyvinylimidazole-based copolymer can be a polyvinylimidazole-co-polystyrene polymer. In certain embodiments, the polyvinylimidazole-co-polystyrene polymer can be a poly(N-vinylimidazole)-co-polystyrene polymer, a poly(1-vinylimidazole)-co-polystyrene polymer, or a derivative thereof.

In certain embodiments, the copolymer of the drug delivery composition disposed upon the analyte sensor is a polyvinylpyridine-based copolymer. In certain embodiments, the polyvinylpyridine-based copolymer can be a copolymer of vinylpyridine and styrene or a derivative thereof.

In certain embodiments, the polyvinylpyridine-based copolymer can be a polyvinylpyridine-co-polystyrene polymer. In certain embodiments, the polyvinylpyridine-co-polystyrene polymer can be a poly(4-vinylpyridine)-co-polystyrene polymer, a poly(2-vinylpyridine)-co-polystyrene polymer, or a derivative thereof. In certain embodiments, the polyvinylpyridine-co-polystyrene polymer is a poly(4-vinylpyridine)-co-polystyrene polymer

In certain embodiments, the polyvinylpyridine-co-polystyrene polymer can include 1-50 mer % of styrene units. In certain embodiments, the polyvinylpyridine-co-polystyrene polymer can include 1-40 mer % of styrene units. In certain embodiments, the polyvinylpyridine-co-polystyrene polymer can include 1-30 mer % of styrene units.

In certain embodiments, a weight average molecular weight of the copolymer is in a range of about 5 kD-1,000 kD.

In certain embodiments, the crosslinker can be a diglycidyl- or triglycidyl-functional epoxy.

In certain embodiments, the crosslinker can be selected from the group consisting of diglycidyl-PEG (200-1000), glycerol triglycidyl ether, and a combination thereof.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, mol % crosslinking of the copolymer can be in a range of about 0.1 mol %-50 mol %. In certain embodiments, mol % crosslinking of the copolymer can be in a range of about 1 mol %-50 mol %. In certain embodiments, mol % crosslinking of the copolymer can be in a range of about 0.1 mol %-40 mol %. In certain embodiments, mol % crosslinking of the copolymer can be in a range of about 1 mol %-40 mol %. In certain embodiments, mol % crosslinking of the copolymer can be in a range of about 0.1 mol %-30 mol %. In certain embodiments, mol % crosslinking of the copolymer can be in a range of about 1 mol %-30 mol %. In certain embodiments, mol % crosslinking of the copolymer can be in a range of about 0.1 mol %-10 mol %. In certain embodiments, mol % crosslinking of the copolymer can be in a range of about 1 mol %-10 mol %.

In certain embodiments, the therapeutic agent can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and combinations thereof.

In certain embodiments, the therapeutic agent can be an anti-inflammatory agent. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. In certain embodiments, the anti-inflammatory agent is dexamethasone or a derivative or a salt form thereof. In certain embodiments, the derivative and/or salt form of dexamethasone is dexamethasone acetate. In certain embodiments, the derivative and/or salt form of dexamethasone is dexamethasone sodium phosphate.

In certain embodiments, the drug delivery composition can include an amount of the therapeutic agent in a range of 0.01 wt %-50 wt % based on the total weight of the copolymer. In certain embodiments, the drug delivery composition can include an amount of the therapeutic agent in a range of 0.01 wt %-40 wt % based on the total weight of the copolymer.

In certain embodiments, the crosslinker is bonded to the hydrophilic unit of the copolymer to form a charge.

In certain embodiments, the therapeutic agent is not covalently bound to the copolymer.

In certain embodiments, the therapeutic agent is covalently bound to the copolymer.

In certain embodiments, the drug delivery composition can continuously release the therapeutic agent at a set or predetermined drug delivery rate for a set or predetermined days such as for at least 30 days.

In certain embodiments, the mass transport limiting membrane disposed upon an analyte sensor can include a polyvinylpyridine (e.g., poly(4-vinylpyridine) or poly(a-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and/or the like.

For a more detailed description drug delivery compositions that can be included in an analyte sensor and the features of such drug delivery compositions, reference can be made to the relevant part of the drug delivery composition disclosed above, e.g., in Section III above. For a more detailed description of the analyte sensor, reference can be made to the relevant part of the analyte sensor disclosed above.

### V. Delivery Devices and Methods of Delivery

The present disclosure further provides devices for delivering a drug delivery composition disclosed herein, for delivering of an analyte sensor disclosed herein and for simultaneously delivering a drug delivery composition and an analyte sensor disclosed herein. The present disclosure further provides methods for delivering a drug delivery composition disclosed herein, for delivering of an analyte sensor disclosed herein and for simultaneously delivering a drug delivery composition and an analyte sensor disclosed herein. The present disclosure further provides methods of controlling a drug delivery rate of an analyte sensor such as a subcutaneous sensor.

In certain embodiments, a method of the present disclosure can include providing a drug delivery composition disclosed herein and implanting the drug delivery composition (e.g., subcutaneously) into a subject. In certain embodiments, methods of the present disclosure can include providing an analyte sensor disclosed herein (e.g., that comprises a drug delivery composition) and implanting the analyte sensor (e.g., subcutaneously) into a subject. For example, but not by way of limitation, the analyte sensor and/or drug delivery composition can be implanted into a subject through the use of a sharp.

In certain embodiments, the present disclosure provides sharps including an analyte sensor and/or a drug delivery composition described herein. For example, but not by way of limitation, certain embodiments of the present disclosure are directed to a sharp, such as a pre-loaded sharp for delivering a drug delivery composition. FIG. 4 illustrates a cross-sectional diagram of an exemplary sharp according to certain embodiments of the present disclosure. As shown in FIG. 4, in certain embodiments, the sharp 401′ can include an analyte sensor 403′ and a drug delivery composition 402′ (e.g., where the drug delivery composition includes (i) a copolymer including a plurality of copolymer chains, wherein each of the plurality of copolymer chains comprises a backbone including a plurality of hydrophilic units and a plurality of hydrophobic units, (ii) a crosslinker crosslinking at least a portion of the hydrophilic units between respective copolymer chains, and (iii) a therapeutic agent). In certain embodiments, the sharp can further include an analyte sensor, where the analyte sensor is positioned within a channel 404′ of the sharp and the drug delivery composition is positioned distally to the analyte sensor within the channel 404′ of the sharp.

In certain embodiments, the drug delivery composition is of a shape and/or size that fits within the dimensions of the sharp (i.e., insertion needle) used to deliver the drug delivery composition (e.g., in close proximity to the analyte sensor). For example, but not by way of limitation, the drug delivery composition is of a shape that corresponds to a lumen, channel or groove of the sharp. In certain embodiments, the drug delivery composition has a shape that allows it to fit securely within a lumen, channel or groove of a delivery device, e.g., a sharp, during shipping but also allows for release of the drug delivery composition from the delivery device into a tissue. In certain embodiments, the drug delivery composition has a cube shape, rectangular shape, cylindrical shape, sphere shape, diamond shape or an irregular shape. As shown in FIG. 4, the drug delivery composition 402′ can have a shape that fits within the U-shaped channel 404′ of an exemplary sharp 401′. Alternatively, the drug delivery composition can have a sphere or cylindrical shape to fit with a cylindrical channel of a sharp. In certain embodiments, the drug delivery composition unit can split in more than one piece upon contacting tissue.

In certain embodiments, the sharp used to deliver the drug delivery composition can be the sharp used to deliver an analyte sensor transcutaneously under a user's skin. For example, but not by way of limitation, the drug delivery composition can be deployed in a tissue of a user at the same time as the analyte sensor. As shown in FIG. 4, the drug delivery composition 402′ can be placed in a lumen, channel or groove at the distal tip of a sharp 401′ in front of an analyte sensor 403′. During the insertion process of the analyte sensor, the movement of the analyte sensor 403′ out of the distal tip of the sharp 401′ can force the drug delivery composition 402′ from the sharp 401′ and into the tissue of the user in close proximity to the analyte sensor in vivo.

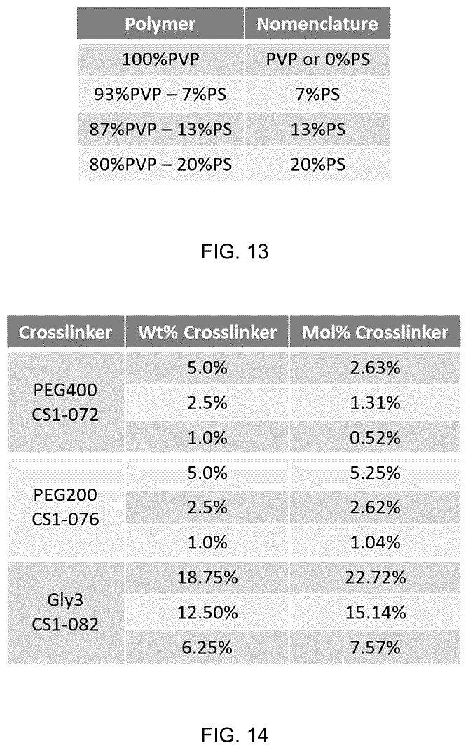

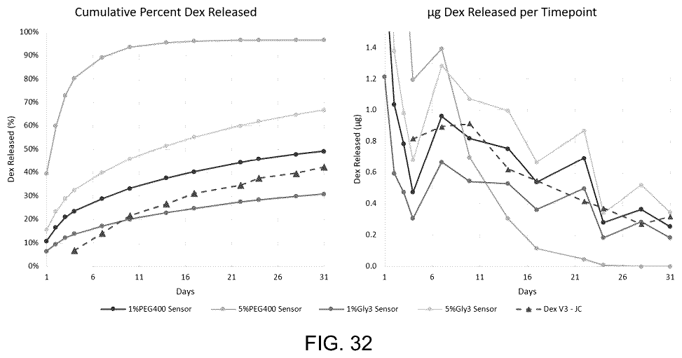

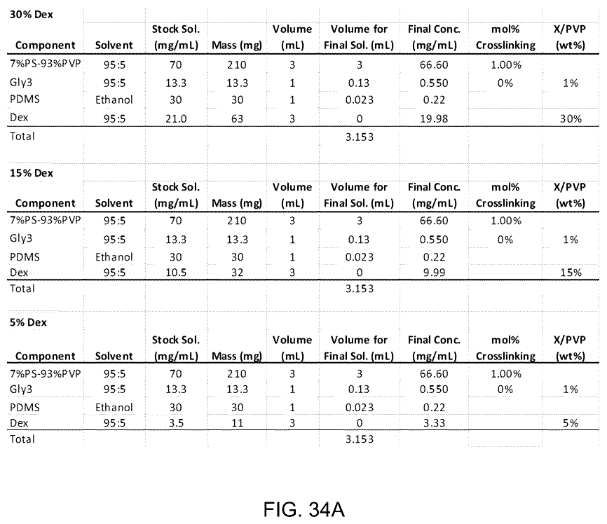

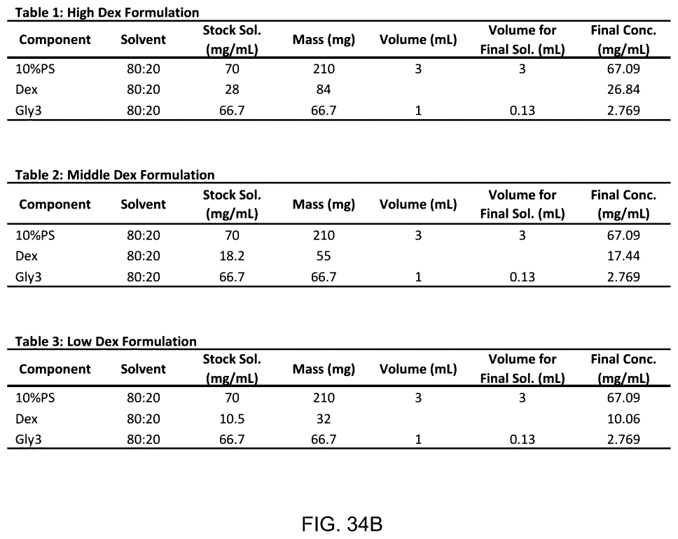

In certain embodiments, the sharp is a part of an introducer as disclosed herein. In certain embodiments, the sharp is a part of a sharp module and/or sensor applicator, e.g., as disclosed in International Publication Nos. WO 2018/136898, WO 2019/236859 and WO 2019/236876 and U.S. Patent Publication No. 2020/0196919, each of which is incorporated by reference in its entirety herein. For example, but not by way of limitation, the sharp can be a part of a sensor applicator as shown in FIG. 32B (e.g., the sharp is noted as 3216), FIG. 34B (e.g., the sharp is noted as 3216), FIG. 40B (e.g., the sharp is noted as 3908) and FIG. 113 (e.g., the sharp is noted as 11308) of WO 2019/236859. In certain embodiments, the sharp can be a part of a sensor module as shown in FIG. 13 of WO 2019/236876 (e.g., the sharp (1318) is incorporated into a sensor module (noted as 1314) for insertion of a sensor (1316)).

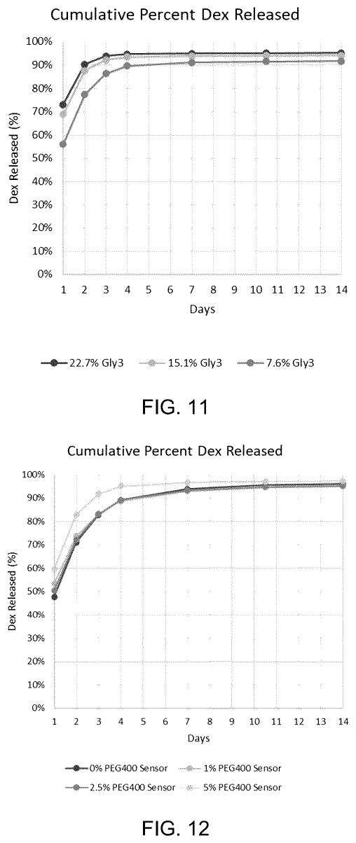

Further details regarding non-limiting embodiments of applicators, their components and variants thereof, are described in U.S. Patent Publication Nos. 2013/0150691, 2016/0331283 and 2018/0235520, all of which are incorporated by reference herein in their entireties and for all purposes. In certain embodiments, the sharp is part of a sensor applicator as shown in FIG. 11A of U.S. 2013/0150691 (e.g., the sharp is shown as 1030 and the sensor supported within the sharp is noted as 1102). Further details regarding non-limiting embodiments of sharp modules, sharps, their components and variants thereof, are described in U.S. Patent Publication No. 2014/0171771, which is incorporated by reference herein in its entirety and for all purposes.

The present disclosure further provides a sharp that includes the drug delivery composition. In certain embodiments, the sharp can include a channel that includes a drug delivery composition retained within the channel. In certain embodiments, the drug delivery composition is located within the channel at the distal tip of the sharp. In certain embodiments, the sharp can further include an analyte sensor retained within the channel. In certain embodiments, both the drug delivery composition and analyte sensor are retained within a channel of the sharp, where the drug delivery composition is located distal to the analyte sensor within the channel of the sharp as shown in FIG. 4.

In certain embodiments, the pre-loaded sharp can be used in a method to deliver the drug delivery composition near the analyte sensor in vivo. For example, but not by way of limitation, the method can include providing a sharp that includes (a) an analyte sensor and (b) a drug delivery composition, where the analyte sensor is positioned within a channel of the sharp, and where the drug delivery composition is positioned distally to the analyte sensor within the channel of the sharp. In certain embodiments, the method can further include penetrating a tissue of a subject with the sharp and inserting the drug delivery composition and analyte sensor into the tissue of the subject. In certain embodiments, the method includes retracting the sharp from the tissue of the subject to retain the drug delivery composition and analyte sensor in the tissue of the subject.

In certain embodiments, the present disclosure further provides methods for controlling a drug delivery rate of an analyte sensor comprising a therapeutic agent. In certain embodiments, the method of controlling a drug delivery rate of an analyte sensor such as a subcutaneous sensor can include: (i) providing a sharp including an analyte sensor comprising a drug delivery composition according to certain embodiments of the present disclosure, (ii) penetrating a tissue of a subject with the sharp, (iii) inserting the analyte sensor into the tissue of the subject and (iv) retracting the sharp from the tissue of the subject. In certain embodiments, the sharp can include a second drug delivery composition positioned distally to the analyte sensor within the channel of the sharp.

In certain embodiments, the analyte sensor provided in the sharp, and delivered by the disclosed methods, can be any analyte sensor disclosed herein, e.g., an analyte sensor that includes a therapeutic agent. In certain embodiments, the therapeutic agent provided in the drug delivery composition can be different from the therapeutic agent incorporated into the analyte sensor. Alternatively, the therapeutic agent provided in the drug delivery composition can be the same as the therapeutic agent incorporated into the analyte sensor. For example, but not by way of limitation, the therapeutic agent provided in the drug delivery composition and the therapeutic agent incorporated into the analyte sensor can both be dexamethasone.

Non-limiting examples of analyte sensors that can be delivered by a sharp are disclosed in Section IV. In certain embodiments, the analyte sensor is a subcutaneous sensor such as a subcutaneously implanted sensor. In certain embodiments, the analyte sensor is a dermal sensor. In certain embodiments, the analyte sensor is an intravenous sensor such as intravenously implanted sensor. In certain embodiments, the analyte sensor is configured to detect glucose. In certain embodiments, the analyte sensor is configured to detect glucose and ketones. In certain embodiments, the analyte sensor is configured to detect lactate. In certain embodiments, the analyte sensor is configured to detect creatinine. In certain embodiments, the analyte sensor is configured to detect alcohol.

Non-limiting examples of drug delivery compositions that can be delivered by a sharp and/or incorporated into an analyte sensor are disclosed in Section III. For example, but not by way of limitation, the hydrophilic unit of the copolymer present within the drug delivery composition can include a nitrogen-containing heterocyclic unit such as a pyridine unit, a pyridazine unit, a pyrimidine unit, a pyrazine unit, a triazine unit, an imidazole unit, a pyrazole unit, etc. In certain embodiments, the hydrophobic unit of the copolymer present within the drug delivery composition can include a non-heteroatom containing aromatic unit such as a benzene (phenyl) unit, a naphthalene unit, an anthracene unit, etc., an acyclic aliphatic unit such as a straight or branched alkyl unit, a straight or branched alkenyl unit, a straight or branched alkynyl unit, etc., and/or a cyclic aliphatic unit such as a cyclobutyl, a cyclopentyl unit, a cyclohexyl unit, a cycloheptyl unit, a cyclooctyl unit, a cyclohexenyl unit, etc.

In certain embodiments, the copolymer can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.

In certain embodiments, the copolymer can include a block polymer.

In certain embodiments, the polyvinylimidazole-based copolymer can be a copolymer of vinylimidazole and styrene or a derivative thereof.

In certain embodiments, the polyvinylimidazole-based copolymer can be a polyvinylimidazole-co-polystyrene polymer. In certain embodiments, the polyvinylimidazole-co-polystyrene polymer can be a poly(N-vinylimidazole)-co-polystyrene polymer, a poly(1-vinylimidazole)-co-polystyrene polymer, or a derivative thereof.

In certain embodiments, the polyvinylpyridine-based copolymer can be a copolymer of vinylpyridine and styrene or a derivative thereof.

In certain embodiments, the polyvinylpyridine-based copolymer can be a polyvinylpyridine-co-polystyrene polymer. In certain embodiments, the polyvinylpyridine-co-polystyrene polymer can be a poly(4-vinylpyridine)-co-polystyrene polymer, a poly(2-vinylpyridine)-co-polystyrene polymer, or a derivative thereof.

In certain embodiments, the polyvinylpyridine-co-polystyrene polymer can include 1-50 mer % of styrene units. In certain embodiments, the polyvinylpyridine-co-polystyrene polymer can include 1-40 mer % of styrene units. In certain embodiments, the polyvinylpyridine-co-polystyrene polymer can include 1-30 mer % of styrene units.

In certain embodiments, a weight average molecular weight of the copolymer is in a range of about 5 kD-1,000 kD.

In certain embodiments, the crosslinker can be a diglycidyl- or triglycidyl-functional epoxy.

In certain embodiments, the crosslinker can be selected from the group consisting of diglycidyl-PEG (200-1000), glycerol triglycidyl ether, and a combination thereof.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, mol % crosslinking of the copolymer can be in a range of about 0.1 mol %-50 mol %. In certain embodiments, mol % crosslinking of the copolymer can be in a range of about 1 mol %-50 mol %. In certain embodiments, mol % crosslinking of the copolymer can be in a range of about 0.1 mol %-40 mol %. In certain embodiments, mol % crosslinking of the copolymer can be in a range of about 1 mol %-40 mol %. In certain embodiments, mol % crosslinking of the copolymer can be in a range of about 0.1 mol %-30 mol %. In certain embodiments, mol % crosslinking of the copolymer can be in a range of about 1 mol %-30 mol %. In certain embodiments, mol % crosslinking of the copolymer can be in a range of about 0.1 mol %-10 mol %. In certain embodiments, mol % crosslinking of the copolymer can be in a range of about 1 mol %-10 mol %.

In certain embodiments, the therapeutic agent can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and combinations thereof.

In certain embodiments, the therapeutic agent can be an anti-inflammatory agent. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. In certain embodiments, the anti-inflammatory agent is dexamethasone or a derivative or a salt form thereof. In certain embodiments, the derivative of dexamethasone is dexamethasone acetate. In certain embodiments, the derivative of dexamethasone is dexamethasone sodium phosphate.

In certain embodiments, the drug delivery composition can include an amount of the therapeutic agent in a range of 0.01 wt %-50 wt % based on the total weight of the copolymer. In certain embodiments, the drug delivery composition can include an amount of the therapeutic agent in a range of 0.01 wt %-40 wt % based on the total weight of the copolymer.

In certain embodiments, the drug delivery composition can include about 0.1 μg to about 200 μg of the therapeutic agent. In certain embodiments, the drug delivery composition can include about 0.1 μg to about 20 μg of the therapeutic agent. In certain embodiments, the drug delivery composition can include about 0.1 μg to about 10 μg of the therapeutic agent.

In certain embodiments, the crosslinker is bonded to the hydrophilic unit of the copolymer to form a charge.

In certain embodiments, the therapeutic agent is not covalently bound to the copolymer.

In certain embodiments, the therapeutic agent is covalently bound to the copolymer.

In certain embodiments, the drug delivery composition continuously releases the therapeutic agent at a set or predetermined drug delivery rate for a set or predetermined days such as for at least 30 days.

In certain embodiments, the analyte sensor is configured to detect glucose.

For more detailed description of the drug delivery composition, reference can be made to the relevant part of the drug delivery composition disclosed above, e.g., Section III above. For more detailed description of the analyte sensor, reference can be made to the relevant part of the analyte sensor disclosed above, e.g., Section IV above.

### VI. Exemplary Embodiments

A. In certain non-limiting embodiments, the presently disclosed subject matter provides a drug delivery composition, comprising:
(i) a copolymer comprising a plurality of copolymer chains, wherein each of the plurality of copolymer chains comprises a backbone comprising a plurality of hydrophilic units and a plurality of hydrophobic units;
(ii) a crosslinker crosslinking at least a portion of the hydrophilic units between respective copolymer chains; and
(iii) a therapeutic agent.

A1. The drug delivery composition of A, wherein (i) the hydrophilic unit is selected from among a pyridine unit, a pyridazine unit, a pyrimidine unit, a pyrazine unit, a triazine unit, an imidazole unit, and a pyrazole unit, and/or (ii) the hydrophobic unit is selected from among a non-heteroatom containing aromatic unit, an acyclic aliphatic unit, and a cyclic aliphatic unit.

A1-1. The drug delivery composition of A1, wherein the hydrophilic unit is a pyridine unit.

A1-2. The drug delivery composition of A1, wherein the hydrophobic unit is an aromatic unit.

A2. The drug delivery composition of A-A1-2, wherein the copolymer is selected from the group consisting of a polyvinylpyridine-based copolymer, a polyvinylimidazole-based copolymer, and a combination thereof.

A2-1. The drug delivery composition of A2, wherein the copolymer is a polyvinylpyridine-based copolymer.

A3. The drug delivery composition of A2 or A2-1, wherein the polyvinylpyridine-based copolymer is a polyvinylpyridine-co-polystyrene polymer.

A4. The drug delivery composition of A3, wherein the polyvinylpyridine-co-polystyrene polymer comprises about 1-50 mer % of styrene units.

A5. The drug delivery composition of A3 or A4, wherein the polyvinylpyridine-co-polystyrene polymer comprises about 1-30 mer % of styrene units.

A6. The drug delivery composition of A-A5, wherein a weight average molecular weight of the copolymer is in a range of about 5 kD to about 1000 kD.

A7. The drug delivery composition of A-A6, wherein the crosslinker is a diglycidyl- or triglycidyl-functional epoxy.

A8. The drug delivery composition of A7, wherein the crosslinker is selected from the group consisting of diglycidyl-PEG (200-1000), glycerol triglycidyl ether, and a combination thereof.

A9. The drug delivery composition of A8, wherein the crosslinker is selected from the group consisting of diglycidyl-PEG 200, diglycidyl-PEG 400, glycerol triglycidyl ether, and a combination thereof.

A10. The drug delivery composition of A-A9, wherein mol % crosslinking of the copolymer is in a range of about 0.1 mol %-about 50 mol %.

A10-1. The drug delivery composition of A10, wherein mol % crosslinking of the copolymer is in a range of about 1 mol %-about 50 mol %.

A11. The drug delivery composition of A-A10, wherein the mol % crosslinking of the copolymer is in a range of about 0.2 mol %-about 30 mol %.

A11-1. The drug delivery composition of A-A10, wherein the mol % crosslinking of the copolymer is in a range of about 1 mol %-about 30 mol %.

A12. The drug delivery composition of A-A11-1, wherein the therapeutic agent is at least one selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.

A13. The drug delivery composition of A-A12, wherein the mol % crosslinking of the copolymer is in a range of about 0.5 mol %-about 10 mol %.

A13-1. The drug delivery composition of A-A12, wherein the mol % crosslinking of the copolymer is no greater than about 20 mol %.

A14. The drug delivery composition of A-A13-1, wherein the therapeutic agent is an anti-inflammatory agent.

A15. The drug delivery composition of A14, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, derivatives thereof, salts form thereof, and combinations thereof.

A16. The drug delivery composition of A-A15, wherein the anti-inflammatory agent is dexamethasone, a derivative thereof, or a salt form thereof.

A17. The drug delivery composition of A-A16, wherein the drug delivery composition comprises the therapeutic agent in a range of about 0.01 wt %-about 40 wt % based on a weight of the copolymer.

A18. The drug delivery composition of A-A17, wherein the crosslinker is bonded to the hydrophilic unit of the copolymer to form a charge.

A19. The drug delivery composition of A-A18, wherein the drug delivery composition comprises from about 0.1 μg to about 200 μg of the therapeutic agent.

A20. The drug delivery composition of A-A19, wherein the drug delivery composition comprises from about 0.1 μg to about 20 μg of the therapeutic agent.

A21. The drug delivery composition of A-A20, wherein the drug delivery composition comprises from about 0.1 μg to about 10 μg of the therapeutic agent.

A22. The drug delivery composition of A-A21, wherein the drug delivery composition comprises from about 0.1 μg to about 5 μg of the therapeutic agent.

A23. The drug delivery composition of A-A22, wherein the therapeutic agent is not covalently bonded to the copolymer.

B. In certain non-limiting embodiments, the presently disclosed subject matter provides an analyte sensor, comprising:
(i) a sensor tail comprising at least a first working electrode;
(ii) an active area upon a surface of the first working electrode for detecting an analyte;
(iii) a mass transport limiting membrane permeable to the analyte that overcoats at least the active area;
(iv) a counter/reference electrode; and
(v) the drug delivery composition of any one of A-A23.

B1. The analyte sensor of B, wherein the drug delivery composition is disposed upon the counter/reference electrode, the working electrode or the mass transport limiting membrane.

B2. The analyte sensor of B or B1, wherein the drug delivery composition is disposed upon the counter/reference electrode.

B3. The analyte sensor of B or B1, wherein the drug delivery composition is disposed upon the working electrode.

B4. The analyte sensor of B or B1, wherein the drug delivery composition is disposed upon the mass transport limiting membrane.

C. In certain non-limiting embodiments, the presently disclosed subject matter provides a method of controlling a drug delivery rate of an analyte sensor and/or implanting an analyte sensor in a subject, the method comprising:
(i) providing the analyte sensor of any one of B-B4; and
(ii) implanting the analyte sensor subcutaneously.

D. In certain non-limiting embodiments, the presently disclosed subject matter provides a method of controlling a drug delivery rate of an analyte sensor and/or implanting an analyte sensor in a subject, the method comprising:
(i) providing a sharp comprising an analyte sensor and the drug delivery composition of any one of A-A23;
(ii) penetrating a tissue of the subject with the sharp;
(iii) inserting the drug delivery composition and the analyte sensor into the tissue of the subject; and
(iv) retracting the sharp from the tissue of the subject.

E. In certain non-limiting embodiments, the presently disclosed subject matter provides a sharp, comprising a drug delivery composition of any one of A-A23.

E1. The sharp of E, further comprising an analyte sensor, wherein the analyte sensor is positioned within a channel of the sharp and the drug delivery composition is positioned distally to the analyte sensor within the channel of the sharp.

E2. The sharp of E or E1, wherein the analyte sensor can comprise a drug delivery composition of any one of A-A23.

F. In certain non-limiting embodiments, the presently disclosed subject matter provides a method of manufacturing a drug delivery composition, the method comprising:
(i) providing a copolymer comprising a plurality of copolymer chains, wherein each of the plurality of copolymer chains comprises a backbone comprising a plurality of hydrophilic units and a plurality of hydrophobic units;
(ii) applying a crosslinker and a therapeutic agent to the copolymer; and
(iii) crosslinking the crosslinker to at least a portion of the hydrophilic units between respective copolymer chains.

### EXAMPLES

### Example 1: Incorporation of a Drug Delivery Composition into an Analyte Sensor

This example provides the analysis of polymer-based drug delivery compositions. In example embodiments of the present disclosure, polyvinylpyridine (PVP) or polyvinylpyridine-co-polystyrene (PVP-PS) copolymer was utilized as a representative copolymer of the drug delivery composition; dexamethasone (Dex) was utilized as a representative therapeutic agent of the drug delivery composition; diglycidyl-PEG 400 (hereinafter, PEG 400), diglycidyl-PEG 200 (hereinafter, PEG 200), or glycerol triglycidyl ether (hereinafter Gly3) was utilized as a representative crosslinker of the drug delivery composition.

### A. Sample Fabrication

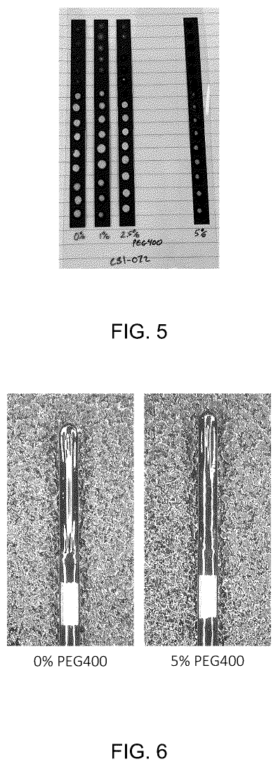

FIG. 5 illustrates exemplary coupons of drug delivery compositions on biocompatible strips (which functions as sensor tail surrogates) according to certain embodiments of the present disclosure. As shown in FIG. 5, for the rapid screening of the formulation of a drug delivery composition, coupons were utilized. For each tested formulation of a drug delivery composition, a 5 μL of the formulation solution of the drug delivery composition was hand-dispensed on a sensor tail surrogate to make test coupons. Multiple coupons were utilized for testing.

FIG. 6 illustrates example sensor tails including drug delivery compositions according to certain embodiments of the present disclosure. For the analyte sensor testing, in certain embodiments, the sensor tail of an analyte sensor was dip-coated with a drug delivery composition. In certain embodiments, a sub-microliter sized drop containing a drug delivery composition was added onto the sensor tail of an analyte sensor using an automated precision liquid dispensing device (e.g., BioDot). In general, the Dex-containing drug delivery composition was deposited onto the sensor tail in a way that did not interfere with the Glucose-Oxidase (GOx) sensing chemistry, or the outer membrane of the analyte sensor.

### B. Measurement Method of Dexamethasone (Dex)

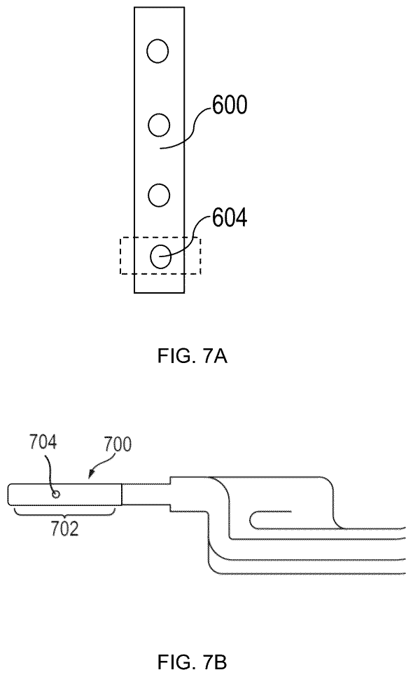

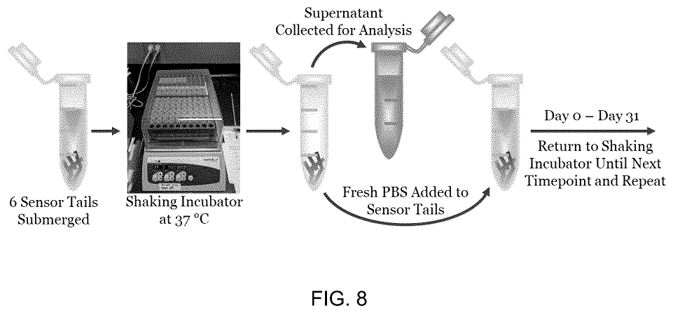

FIGS. 7A-7B illustrate example test samples of a drug delivery composition according to certain embodiments of the present disclosure. FIG. 8 illustrates an example test procedure of a drug delivery composition according to certain embodiments of the present disclosure.

In vitro Dex release of a drug delivery composition was carried out in a pH 7.4 phosphate buffered saline (PBS) solution at 37° C. under agitation, which is similar to a physiological condition.

As shown in FIG. 7A, a test sample 604 was cut from a sensor tail surrogate 600 on which the drug delivery composition was dispensed. As shown in FIG. 7B, a sensor tail 700 including a test sample 704 was cut from a drug loading section 702 of the sensor tail 700 of an analyte sensor. And then, as shown in FIG. 8, several test samples, such as 6 sensor tails, were submerged in the pH 7.4 PBS solution in a vial that was incubated in a shaking incubator at 37° C. under agitation. At each timepoint, such as daily, supernatant was collected for HPLC analysis, a fresh PBS solution was added into the vial, and the vial was returned to the shaking incubator until next timepoint. The steps were repeated up to 31 days.

For the total Dex loading measurement, the test samples were extracted with 100% methanol at 37° C. under agitation for 24 hours to remove all Dex.

The concentration of Dex in the supernatant or in the methanol extraction was measured by utilizing high performance liquid chromatography (HPLC).

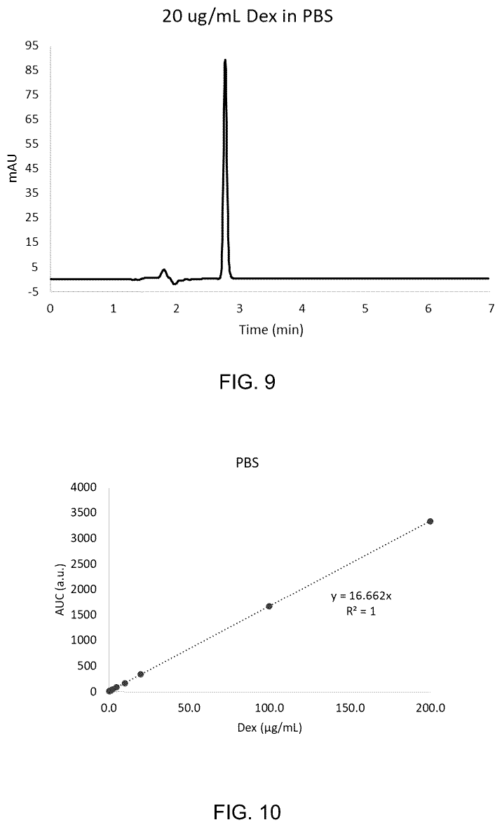

FIG. 9 illustrates an HPLC of dexamethasone according to certain embodiments of the present disclosure. FIG. 10 illustrates a calibration curve of dexamethasone according to certain embodiments of the present disclosure. Area under the curve (AUC) of Dex peak shown in FIG. 9 was measured from suitable Dex concentrations to create a calibration curve shown in FIG. 10, which was utilized to measure unknown Dex concentrations.

### C. 100% PVP Matrices

FIG. 11 illustrates drug delivery profiles of drug delivery compositions including 100% polyvinylpyridine, glycerol triglycidyl ether, and dexamethasone according to certain embodiments of the present disclosure. FIG. 12 illustrates drug delivery profiles of drug delivery compositions including 100% polyvinylpyridine, diglycidyl-PEG 400, and dexamethasone according to certain embodiments of the present disclosure.

As shown in FIG. 11 and FIG. 12, even without the crosslinker or with the low concentration of the crosslinker, 100% PVP polymer (i.e., there is no styrene units in the backbone of the polymer) does not retain Dex, and Dex diffuses out of the polymer matrices quickly. Dex is completely released in less than 7 days (FIG. 11 and FIG. 12). When the concentration of the crosslinker increases, the drug delivery rate increases due to the positive charges formed by crosslinking which improves the swellability of the polymer matrices.

### D. Investigation of the Concentration Effects of Polystyrene in the Polyvinylpyridine-Co-Polystyrene Copolymer and the Concentration and Type or Kind Effects of the Crosslinker

Polyvinylpyridine polymer and several polyvinylpyridine-co-polystyrene copolymers were investigated as a copolymer component of the drug delivery composition. FIG. 13 illustrates exemplary tested polymers and copolymers of drug delivery compositions according to certain embodiments of the present disclosure. The investigated polymer and copolymers have a weight average molecular weight of about 175 kD measured by a suitable method such as gel permeation chromatography. The mer % of styrene units (i.e., mer % polystyrene) in a copolymer was measured by nuclear magnetic resonance (NMR) spectroscopy.

FIG. 14 illustrates example crosslinkers of drug delivery compositions according to certain embodiments of the present disclosure. In certain embodiments, diglycidyl-PEG 200 (PEG 200), diglycidyl-PEG 400 (PEG 400), or glycerol triglycidyl ether (Gly 3) was investigated as a crosslinker to crosslink the copolymer with different mol % crosslinking in the drug delivery composition. The mol % crosslinking of the copolymer is calculated based on the weight amount of the crosslinker relative to the total weight of the copolymer by utilizing the following equation:

$$\frac{\begin{matrix}( mg Crosslinker ) \times ( 105 \frac{g}{mol} Pyridine ) \times \\ ( Crosslinker Functionality )\end{matrix}}{( mg Polymer ) \times ( \frac{g}{mol} Crosslinker )} = mol \% crosslinking ,$$

where the crosslinker functionality is the number of reactive crosslinking groups in one crosslinker molecule. For example, the crosslinker functionality of diglycidyl-PEG 200 is 2, the crosslinker functionality of diglycidyl-PEG 400 is 2, and the crosslinker functionality of glycerol triglycidyl ether is 3.

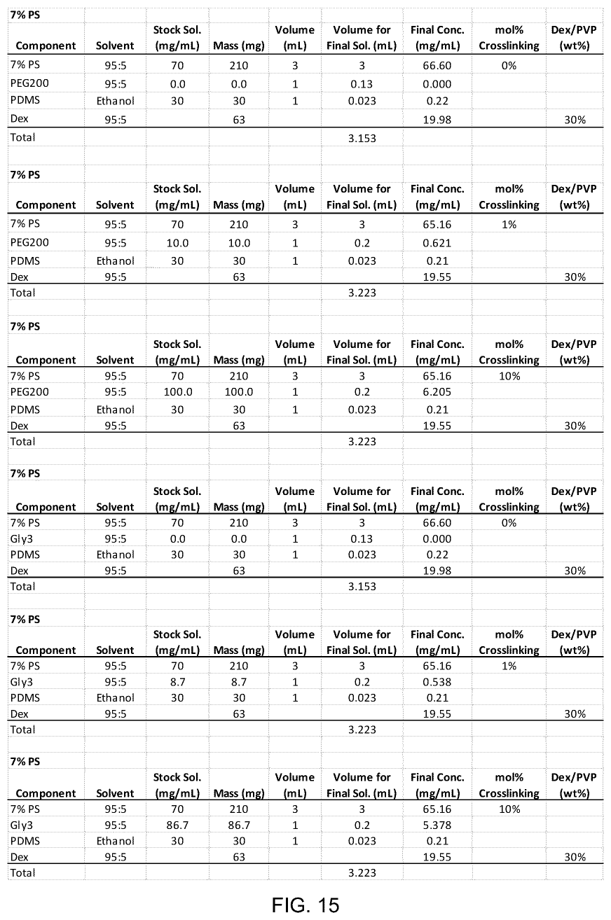

FIG. 15 illustrates exemplary formulations of drug delivery compositions according to certain embodiments of the present disclosure. As shown in FIG. 15, in certain embodiments, 93% PVP-7% PS copolymer was investigated as copolymers in the drug delivery compositions. PEG 200 and Gly3 were investigated as crosslinkers in the drug delivery compositions. The mol % crosslinking of copolymers was 0%, 1 mol %, or 10 mol %. The loading percentage of Dex to the copolymer matrices is 30 wt %. In general, suitable amounts of Dex, the crosslinker, and the copolymer were mixed in a solvent such 95:5 by volume of ethanol and water, and then a 5 μL of the solution was hand-dispensed on a sensor tail surrogate to make a test coupon.

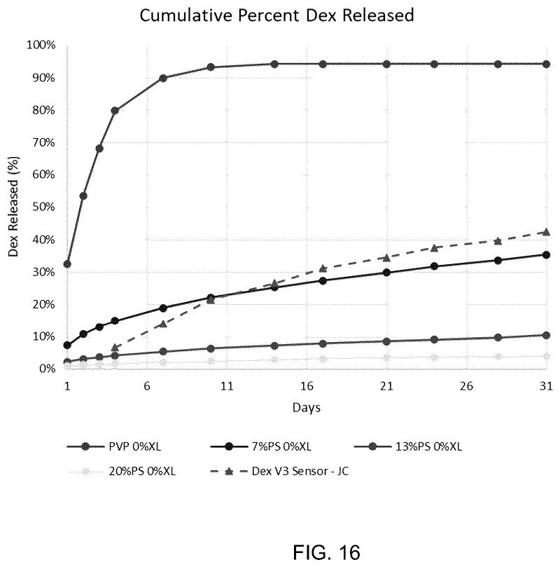

FIG. 16 illustrates drug delivery profiles of drug delivery compositions according to certain embodiments of the present disclosure. As shown in FIG. 16, 100% PVP is too hydrophilic, Dex is quickly released from the polymer matrices and almost completely released less than 7 days. In contrast, the Dex release from 20% PS copolymer without a crosslinker (referred to as “XL”) is too slow. By adjusting the mer % of styrene units in the copolymer, the Dex release rate (i.e., drug delivery rate) can be adjusted, and Dex can be continuously released for at least 31 days. The Dex V3 sensor is a representative sensor that includes a Dex composition, where the Dex is conjugated to the polymer within the composition.

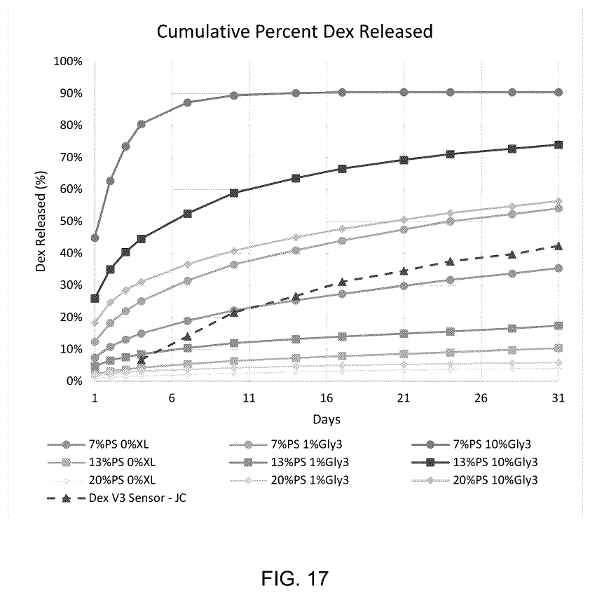

FIG. 17 illustrates drug delivery profiles of drug delivery compositions according to certain embodiments of the present disclosure. As shown in FIG. 17, when the mer % of styrene units in the copolymer increases, and the mol % crosslinking of the copolymer keeps constant, the Dex release rate decreases; when the mer % of styrene units in the copolymer is kept constant, and the mol % crosslinking of the copolymer increases, the Dex release rate increases.

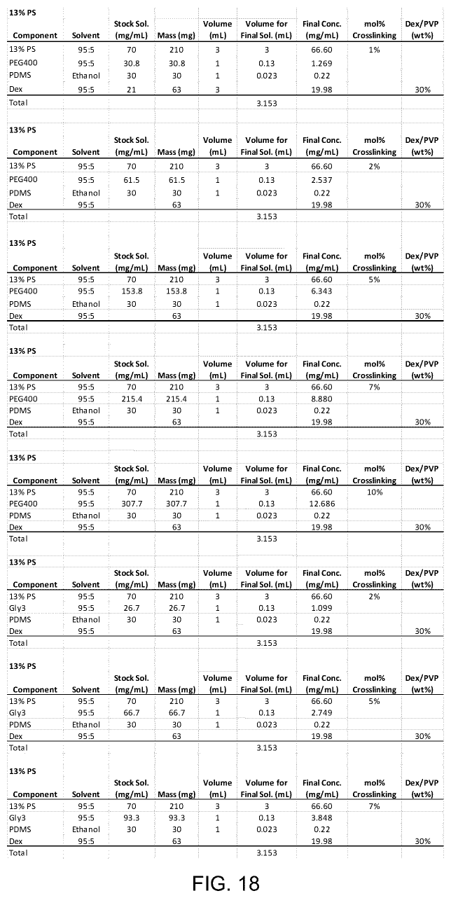

FIG. 18 illustrates exemplary formulations of drug delivery compositions according to certain embodiments of the present disclosure. As shown in FIG. 18, in certain embodiments, 87% PVP-13% PS copolymer was investigated as a polymer in the drug delivery compositions. PEG 400 and Gly3 were investigated as crosslinkers in the drug delivery compositions. The mol % crosslinking of copolymers was 2 mol %, 5 mol %, or 7 mol % in the case of Gly3 as the crosslinker. The mol % crosslinking of copolymers was 1 mol %, 2 mol %, 5 mol %, 7 mol %, or 10 mol % in the case of PEG 400 as the crosslinker. The loading percentage of Dex to the copolymer matrices is 30 wt %. In general, suitable amounts of Dex, the crosslinker, and the copolymer were mixed in a solvent such 95:5 by volume of ethanol and water, and then a 5 μL of the solution was hand-dispensed on a sensor tail surrogate to make a test coupon.

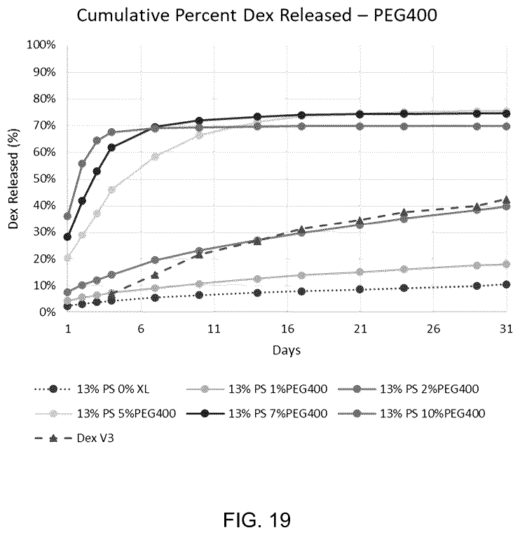

FIG. 19 illustrates drug delivery profiles of drug delivery compositions according to certain embodiments of the present disclosure. As shown in FIG. 19, for a copolymer containing 13% PS copolymer with the crosslinker PEG 400, when the mol % crosslinking of the copolymer increases, the Dex release rate from the copolymer matrices increases.

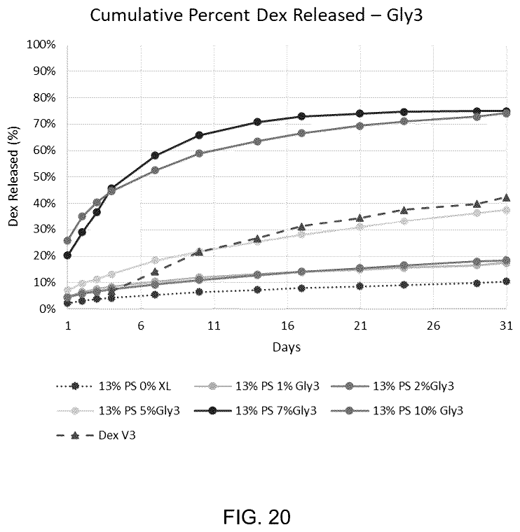

FIG. 20 illustrates drug delivery profiles of drug delivery composition according to certain embodiments of the present disclosure. As shown in FIG. 20, the copolymer is 13% PS copolymer, and the crosslinker is Gly3, when mol % crosslinking of the copolymer matrices increases, the Dex release rate from the copolymer matrices increases.

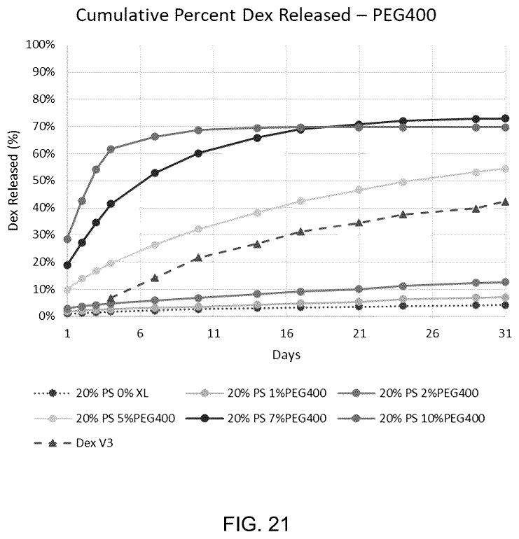

FIG. 21 illustrates drug delivery profiles of drug delivery compositions according to certain embodiments of the present disclosure. As shown in FIG. 21, the copolymer is 20% PS copolymer, and the crosslinker is PEG 400, when the mol % crosslinking of the copolymer matrices increases, the Dex release rate from the copolymer matrices increases.

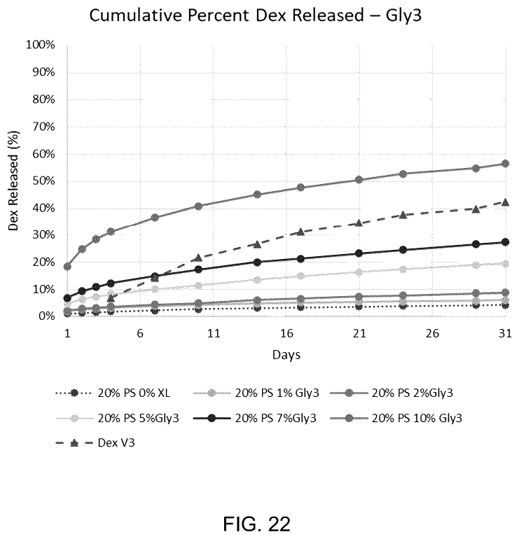

FIG. 22 illustrates drug delivery profiles of drug delivery composition according to certain embodiments of the present disclosure. As shown in FIG. 22, the copolymer is 20% PS copolymer, and the crosslinker is Gly3, when the mol % crosslinking of the copolymer matrices increases, the Dex release rate from the copolymer matrices increases. However, due to the high mer % of styrene units in the copolymer matrices, higher crosslinker amounts are needed to match similar rates demonstrated with copolymers containing lower mer % of styrene.

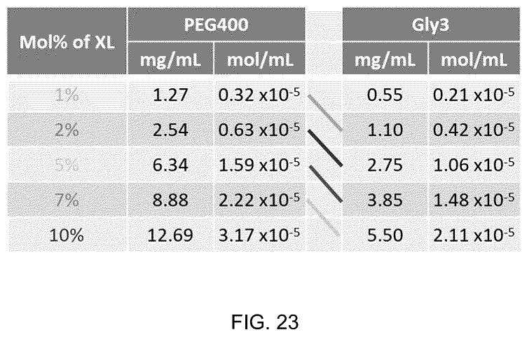

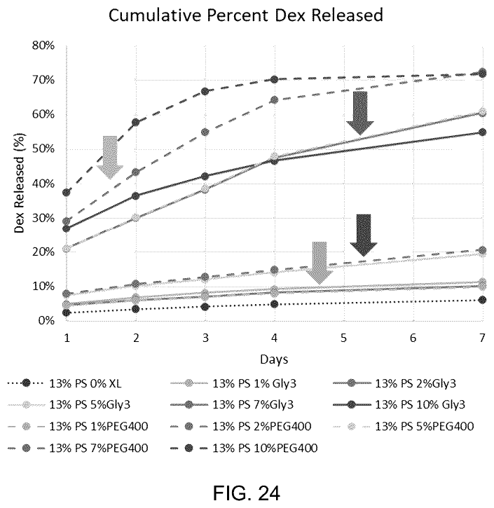

FIG. 23 illustrates concentration relationships between different crosslinkers in drug delivery compositions according to certain embodiments of the present disclosure. FIG. 24 illustrates drug delivery profiles of drug delivery compositions according to certain embodiments of the present disclosure.

As shown in FIG. 23 and FIG. 24, when preparing the drug delivery composition, in order to control the mol % crosslinking of the copolymer, for different crosslinkers, the functionality of the crosslinker, i.e., the number of the reactive crosslinking groups in the crosslinker, has be considered, different weight concentrations of the crosslinker in the drug delivery composition have to be adjusted. After considering the functionality of the crosslinker, PEG 400 and Gly3 show the similar crosslinking effects on the Dex release rate of the drug delivery composition.

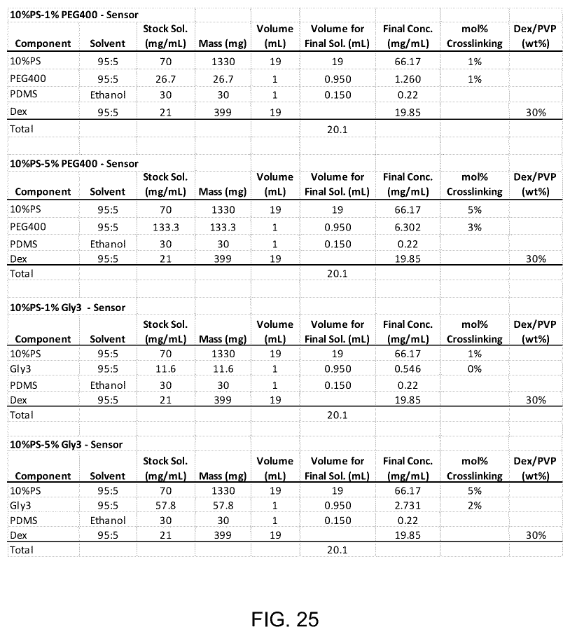

FIG. 25 illustrates exemplary formulations of drug delivery compositions for an analyte sensor according to certain embodiments of the present disclosure. The formulations were applied to the sensor format. As shown in FIG. 25, in certain embodiments, 90% PVP-10% PS copolymer was investigated as a copolymer in the drug delivery compositions on an analyte sensor. PEG 400 and Gly3 were investigated as crosslinkers in the drug delivery compositions. The mol % crosslinking of copolymers was 1 mol % or 5 mol % in the case of Gly3 as the crosslinker. The mol % crosslinking of copolymers was 1 mol % or 5 mol % in the case of PEG 400 as the crosslinker. The loading percentage of Dex to the copolymer matrices is 30 wt %. In general, suitable amounts of Dex, the crosslinker, and the copolymer were mixed in a solvent such 95:5 by volume of ethanol and water, and then a 5 μL of the solution was hand-dispensed on a biocompatible strip to make a test coupon, or the sensor tail of an analyte sensor was dip-coated with the drug delivery composition solution.

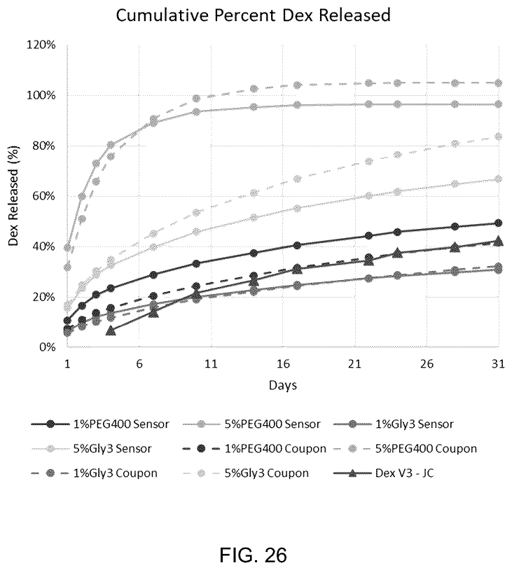

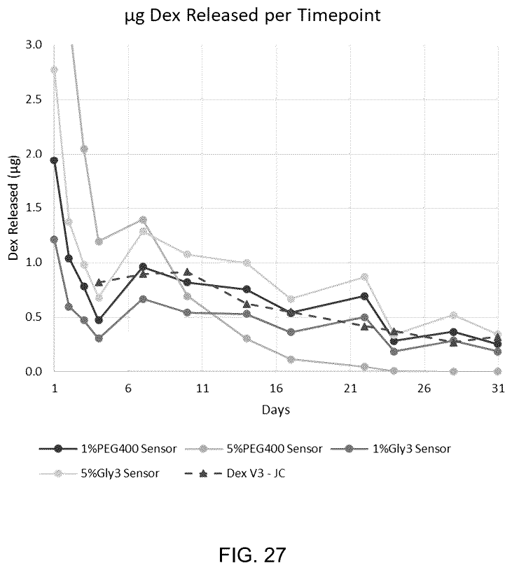

FIG. 26 illustrates drug delivery profiles of drug delivery compositions including 90% PVP-10% PS copolymer on an analyte sensor according to certain embodiments of the present disclosure. FIG. 27 illustrates drug delivery profiles per timepoint of drug delivery compositions including 90% PVP-10% PS copolymer on an analyte sensor according to certain embodiments of the present disclosure. As shown in FIG. 26, for the tested analyte sensors and coupons, when the crosslinker is the same, and mol % crosslinking of the copolymer is the same, the Dex release rate is similar. In addition, when mol % crosslinking of copolymer increases, the Dex release rate increases (FIG. 26). As shown in FIG. 27, the amount of Dex released per sensor per timepoint is in the range of 0.2 μg to 1.5 μg for sensors containing 10% PS and 1% PEG400, 1% Gly3, or 5% Gly3, and Dex can be continuously released for at least 31 days.

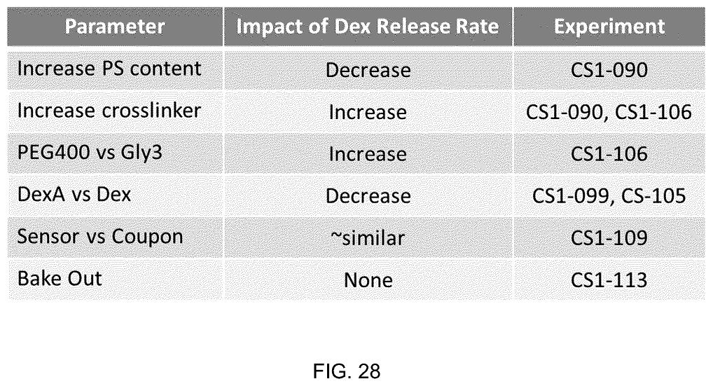

FIG. 28 illustrates impact factors on the drug delivery rate of drug delivery compositions according to certain embodiments of the present disclosure. When the PS content (e.g., amount) of the hydrophobic copolymer increases, i.e., the mer % of styrene units in the copolymer increases, the Dex release rate decreases due to the increasing polymer affinity to Dex. When the amount of the crosslinkers in the drug delivery composition increases, i.e., the mol % crosslinking of the copolymer increases, the Dex release rate increases due to the improved swellability and hydrophilicity of the copolymer matrices. When the same molar amount of PEG 400 and Gly3 are utilized in the drug delivery composition, the Dex release rate increases in the drug delivery composition employing Gly3 as a crosslinker due to higher functionality of Gly3, thus higher mol % crosslinking. When dexamethasone is replaced by dexamethasone acetate (DexA) as the therapeutic agent, the release rate of DexA decreases. This is because the DexA is less polar than Dex and has a stronger non-polar interaction with the styrene unit of the copolymer, which slows down the release of DexA from the copolymer matrices. The Dex release rate of the drug delivery composition on the analyte sensor is similar to the Dex release rate of the drug delivery composition when the drug delivery composition is utilized alone. Bake out (i.e., extended polymer cure time) does not affect the Dex release rate.

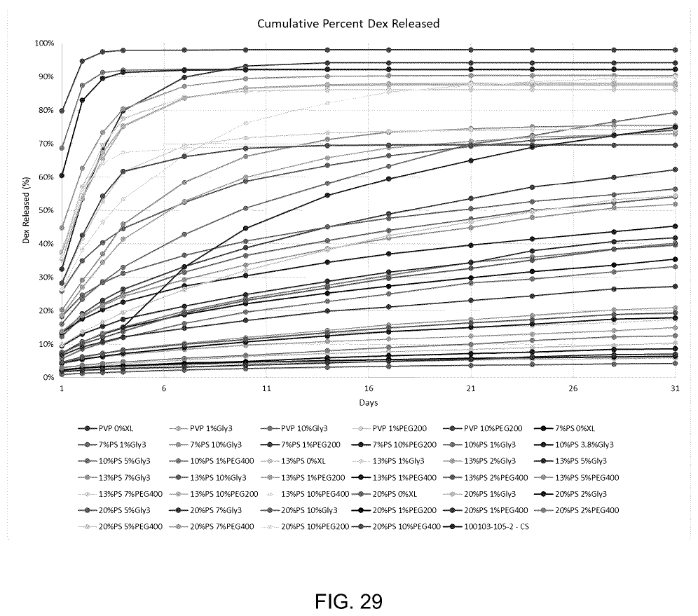

FIG. 29 illustrates drug delivery profiles of drug delivery compositions according to certain embodiments of the present disclosure. As shown in FIG. 29, the Dex release rate can be finely tuned by adjusting the mer % of hydrophobic units (such as styrene units) in the copolymer and/or by adjusting the mol % crosslinking of the copolymer through the crosslinkers such as by adjusting the amount of the crosslinker being utilized and/or the type or kind of the crosslinker being utilized.

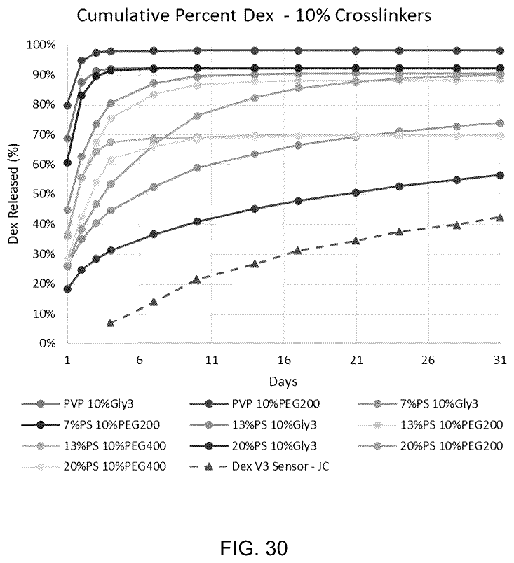

FIG. 30 illustrates drug delivery profiles of drug delivery compositions according to certain embodiments of the present disclosure. As shown in FIG. 30, when the mol % crosslinking of the copolymer is 10 mol %, by adjusting mer % of styrene units in the copolymer, the Dex release rate can be slowed down to deliver a continuous amount of Dex over 31 days by using higher mer % of styrene (13%, 20% PS), or by using a less hydrophilic crosslinker (Gly3).

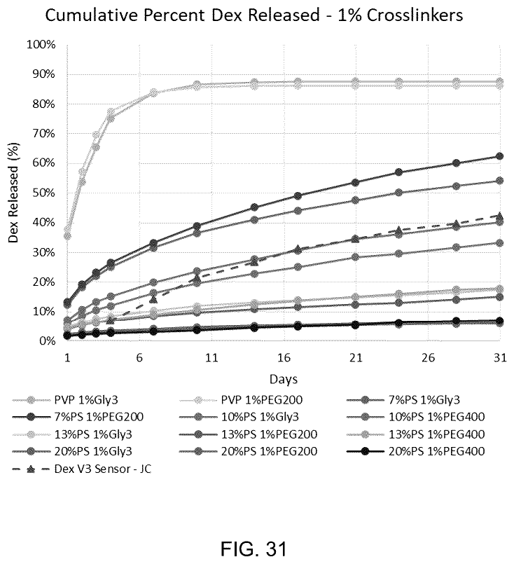

FIG. 31 illustrates drug delivery profiles of drug delivery compositions according to certain embodiments of the present disclosure. As shown in FIG. 31, when the mol % crosslinking of the copolymer is 1%, by adjusting mer % of styrene units in the copolymer, the Dex release rate of the drug delivery composition can be adjusted to deliver Dex continuously over 31 days by adjusting the mer % of styrene (7-20%) across a wide range. Therefore, 1 mol % crosslinking of the copolymer provides a wide range for adjusting the drug delivery rate of the drug delivery composition.

FIG. 32 illustrates drug delivery profiles and drug delivery profiles per timepoint of drug delivery compositions on an analyte sensor according to certain embodiments of the present disclosure. As shown in FIG. 32, for the tested analyte sensors, which employed the drug delivery composition including 90% PVP-10% PS copolymer, when the crosslinker is the same, and mol % crosslinking of copolymer increases, the Dex release rate increases. When PEG 400 is utilized as a crosslinker, 5 mol % crosslinking of copolymer releases a higher amount of dex at earlier time points, and stops releasing Dex after around 23 days. Thus, by adjusting mol % crosslinking within a given copolymer, the desired drug release rate can be achieved.

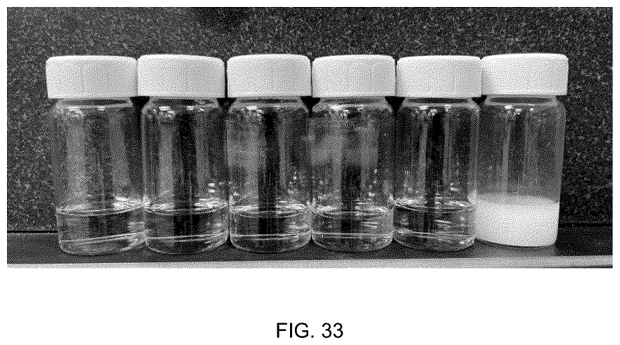

FIG. 33 illustrates the Dex solubility in the polyvinylpyridine-ethanol:water (95:5 by volume) solution according to certain embodiments of the present disclosure. The solubility of Dex in the PVP-ethanol:water solution was measured. For the solutions containing 0 wt %-30 wt % of Dex relative to the weight amount of PVP polymer, after the solutions were vortexed/sonicated within 15 minutes, the solutions became clear. For the solution containing 40 wt % of Dex relative to the weight amount of PVP polymer, the solution became clear after mixing overnight on the nutator. The solution containing 50 wt % of Dex relative to the weight amount of PVP polymer did not become clear after mixing overnight on the nutator. Therefore, the drug delivery composition can include up to 40 wt % of Dex relative to the amount of copolymer to make a clear drug delivery composition.

FIG. 34A illustrates exemplary formulations of drug delivery compositions according to certain embodiments of the present disclosure. As shown in FIG. 34A, the effect of the Dex loading in the drug delivery composition on the Dex release rate was investigated. The copolymer is 93% PVP-7% PS copolymer. The mol % crosslinking of the copolymer is 1 mol % by Gly3. The loading of Dex in the drug delivery composition is 30 wt %, 15 wt %, or 5 wt % relative to the weight amount of copolymer.

FIG. 34B illustrates exemplary formulations of drug delivery compositions that include different percentages of dexamethasone. Table 1 of FIG. 34B shows a drug delivery composition that includes a Dex loading of 40 wt % relative to the weight amount of copolymer. Table 2 of FIG. 34B shows a drug delivery composition that includes a Dex loading of 26 wt % relative to the weight amount of copolymer. Table 3 of FIG. 34B shows a drug delivery composition that includes a Dex loading of 15 wt % relative to the weight amount of copolymer.

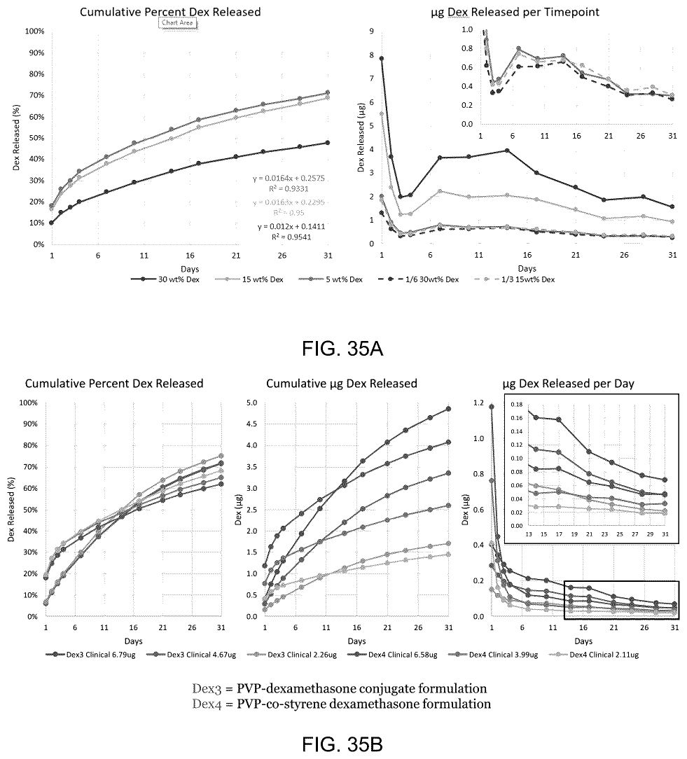

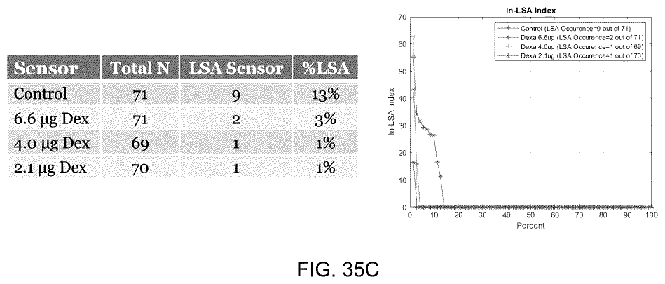

FIG. 35A illustrates drug delivery profiles and drug delivery profiles per timepoint of drug delivery compositions on an analyte sensor according to certain embodiments of the present disclosure. As shown in FIG. 35A, Dex can be continuously released for at least 31 days. For the 5 wt % Dex loading, 70% of Dex is released over 31 days. For the 30 wt % Dex loading, about 50% of Dex is released over 31 days. The amount of Dex daily released by the drug delivery composition containing 30 wt % of Dex is twice the amount of Dex daily released by the drug delivery composition containing 15 wt % of Dex, and six times the amount of Dex daily released by the drug delivery composition containing 5 wt % of Dex, at each timepoint. After normalized with the Dex loading, as shown in the insert panel of the right panel of FIG. 35A, the Dex release rate are similar for all three drug delivery compositions. Therefore, within full solubility, the Dex loading in the drug delivery composition does not impact the Dex release rate.

FIG. 35B illustrates drug delivery profiles and drug delivery profiles per timepoint of drug delivery compositions on an analyte sensor according to certain embodiments of the present disclosure. The drug delivery compositions according to FIG. 34B (10% PS copolymer with 1 mol % Gly3) were deposited onto the counter electrode of sensor tails. As shown in FIG. 35B, Dex can be continuously released for at least 31 days and about 60-75% of the Dex loaded is released by the end of the 31 day period. For the 6.79 μg and 4.67 μg Dex loading, about 70% of Dex is released over 31 days. For the 2.26 μg Dex loading, about 75% of Dex is released over 31 days. For the 6.58 μg Dex loading, about 60% of Dex is released over 31 days. For the 3.99 μg Dex loading, about 65% of Dex is released over 31 days. For the 2.1 μg Dex loading, about 70% of Dex is released over 31 days. For each of the compositions, about 50% of Dex was released between days 13-16.

FIG. 35C shows that incorporating an amount of Dex as low as 2.1 μg in an analyte sensor can reduce LSA significantly. As shown in FIG. 35C, reduction in LSA is similar between an analyte sensor that includes 6.6 μg and an analyte sensor that includes 2.1 μg. LSA was determined by calculating an average and median sensitivity in a rolling 12 hr window if there are >=3 point within the window, and if both average and median sensitivity is less than 80% of stable sensitivity (defined as median sensitivity within hr 10-120) then it is a LSA instance. If there are >=5 LSA instances within 24 hr window and the first instance is more than 24 hr before end of sensor life, then LSA onset time is defined as the time of the first LSA instance. In-LSA Index=Area below 1/(T_EndOfLife−T_LSAOnset). Higher index indicates more severe LSA.

The analyte sensor and/or any other relevant devices or components according to embodiments of the present invention described herein can be implemented utilizing any suitable hardware, firmware (e.g., an application-specific integrated circuit), software, or a combination of software, firmware, and hardware. For example, the various components of the sensor can be formed on one integrated circuit (IC) chip or on separate IC chips. Further, the various components of the sensor can be implemented on a flexible printed circuit film, a tape carrier package (TCP), a printed circuit board (PCB), or formed on one substrate. Further, the various components of the sensor can be a process or thread, running on one or more processors, in one or more computing devices, executing computer program instructions and interacting with other system components for performing the various functionalities described herein. The computer program instructions are stored in a memory which can be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices can be combined or integrated into a single computing device, or the functionality of a particular computing device can be distributed across one or more other computing devices without departing from the scope of the example embodiments of the present invention.

Although the embodiments of the present disclosure have been described, it is understood that the present disclosure should not be limited to these embodiments, but one or more suitable changes and modifications can be made by one ordinary skilled in the art within the spirit and scope of the present disclosure as hereinafter claimed and equivalents thereof.

What is claimed is:

1. A drug delivery composition, comprising:
(i) a copolymer comprising a plurality of copolymer chains, wherein each of the plurality of copolymer chains comprises a backbone comprising a plurality of hydrophilic units and a plurality of hydrophobic units;
(ii) a crosslinker crosslinking at least a portion of the hydrophilic units between respective copolymer chains; and
(iii) a therapeutic agent.

2. The drug delivery composition of claim 1, wherein (a) the hydrophilic unit is selected from the group consisting of a pyridine unit, a pyridazine unit, a pyrimidine unit, a pyrazine unit, a triazine unit, an imidazole unit, and a pyrazole unit, and/or (b) the hydrophobic unit is selected from the group consisting of a non-heteroatom containing aromatic unit, an acyclic aliphatic unit, and a cyclic aliphatic unit.

3. The drug delivery composition of claim 1 or 2, wherein the copolymer is selected from the group consisting of a polyvinylpyridine-based copolymer, a polyvinylimidazole-based copolymer, and a combination thereof.

4. The drug delivery composition of claim 3, wherein the polyvinylpyridine-based copolymer is a polyvinylpyridine-co-polystyrene polymer.

5. The drug delivery composition of claim 4, wherein the polyvinylpyridine-co-polystyrene polymer comprises about 1-50 mer % of styrene units.

6. The drug delivery composition of claim 5, wherein the polyvinylpyridine-co-polystyrene polymer comprises about 1-30 mer % of styrene units.

7. The drug delivery composition of any one of claims 1-6, wherein a weight average molecular weight of the copolymer is in a range of about 5 kD to about 1000 kD.

8. The drug delivery composition of any one of claims 1-7, wherein the crosslinker is a diglycidyl- or triglycidyl-functional epoxy.

9. The drug delivery composition of claim 8, wherein the crosslinker is selected from the group consisting of diglycidyl-PEG (200-1000), glycerol triglycidyl ether, and a combination thereof.

10. The drug delivery composition of claim 9, wherein the crosslinker is selected from the group consisting of diglycidyl-PEG 200, diglycidyl-PEG 400, glycerol triglycidyl ether, and a combination thereof.

11. The drug delivery composition of any one of claims 1-10, wherein mol % crosslinking of the copolymer is in a range of about 0.1 mol %-about 50 mol %.

12. The drug delivery composition of claim 11, wherein the mol % crosslinking of the copolymer is in a range of about 0.1 mol %-about 30 mol %.

13. The drug delivery composition of any one of claims 1-12, wherein the therapeutic agent is at least one selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.

14. The drug delivery composition of claim 13, wherein the therapeutic agent is an anti-inflammatory agent.

15. The drug delivery composition of claim 14, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, derivatives thereof, salts form thereof, and combinations thereof.

16. The drug delivery composition of claim 15, wherein the anti-inflammatory agent is dexamethasone, a derivative thereof, or a salt form thereof.

17. The drug delivery composition of any one of claims 1-16, wherein the drug delivery composition comprises the therapeutic agent in a range of about 0.01 wt %-about 40 wt % based on a weight of the copolymer.

18. The drug delivery composition of any one of claims 1-17, wherein the crosslinker is bonded to the hydrophilic unit of the copolymer to form a charge.

19. An analyte sensor, comprising:
(i) a sensor tail comprising at least a first working electrode;
(ii) an active area upon a surface of the first working electrode for detecting an analyte;
(iii) a mass transport limiting membrane permeable to the analyte that overcoats at least the active area;
(iv) a counter/reference electrode; and
(v) the drug delivery composition of any one of claims 1-18.

20. A method of controlling a drug delivery rate of an analyte sensor, the method comprising:
(i) providing the analyte sensor of claim 19; and
(ii) implanting the analyte sensor subcutaneously.

21. A method of controlling a drug delivery rate of an analyte sensor, the method comprising:
(i) providing a sharp comprising an analyte sensor and the drug delivery composition of any one of claims 1-18;
(ii) penetrating a tissue of a subject with the sharp;
(iii) inserting the drug delivery composition and the analyte sensor into the tissue of the subject; and
(iv) retracting the sharp from the tissue of the subject.

22. A sharp, comprising an analyte sensor and the drug delivery composition of any one of claims 1-18, wherein the analyte sensor is positioned within a channel of the sharp and the drug delivery composition is positioned distally to the analyte sensor within the channel of the sharp.

23. A method of manufacturing a drug delivery composition, the method comprising:
(i) providing a copolymer comprising a plurality of copolymer chains, wherein each of the plurality of copolymer chains comprises a backbone comprising a plurality of hydrophilic units and a plurality of hydrophobic units;
(ii) applying a crosslinker and a therapeutic agent to the copolymer; and
(iii) crosslinking the crosslinker to at least a portion of the hydrophilic units between respective copolymer chains.

24. The analyte sensor of claim 19 for use in controlling a drug delivery rate of the analyte sensor, wherein the analyte sensor is implanted subcutaneously.

25. The drug delivery composition of any one of claims 1-18 for use in controlling a drug delivery rate of an analyte sensor, wherein the drug delivery composition and an analyte sensor are inserted into the tissue of the subject.

26. The drug delivery composition for use of claim 25, wherein the drug delivery composition and the analyte sensor are inserted into the tissue of the subject using a sharp comprising the drug delivery composition and the analyte sensor.

27. The drug delivery composition for use of claim 26, wherein the analyte sensor is positioned within a channel of the sharp and the drug delivery composition is positioned distally to the analyte sensor within the channel of the sharp.
